(19) United States
(12) Patent Application Publication
(10) Pub. No.: US 2019/0100762 A1
(43) Pub. Date: Apr. 4, 2019

(54) NOVEL CAS9 SYSTEMS AND METHODS OF USE

(71) Applicant: PIONEER HI-BRED INTERNATIONAL, INC., JOHNSTON, IA (US)

(72) Inventors: ANDREW MARK CIGAN, MADISON, WI (US); MATTHEW G. KING, JOHNSTON, IA (US); HAINING LIN, CLIVE, IA (US); JOSHUA K. YOUNG, JOHNSTON, IA (US)

(73) Assignee: PIONEER HI-BRED INTERNATIONAL, INC., JOHNSTON, IA (US)

(21) Appl. No.: 16/083,191

(22) PCT Filed: Feb. 27, 2017
(86) PCT No.: PCT/US17/19640

§ 371 Date: Sep. 7, 2018

Related U.S. Application Data
(60) Provisional application No. 62/306,904, filed on Mar. 11, 2016.

(51) Int. Cl. C12N 15/82; C12N 15/113; C12N 9/22

(52) U.S. Cl. CPC C12N 15/8213; C12N 15/113; C12N 2310/20; C12N 9/22; C12N 15/8216; C12N 15/821

(57) ABSTRACT

Compositions and methods are provided for novel Cas9 systems, including, but not limiting to, novel guide polynucleotide/Cas9 endonucleases complexes, single or dual guide RNAs, guide RNA elements, and Cas9 endonucleases. The present disclosure also describes methods for genome modification of a target sequence in the genome of a cell, for gene editing, and for inserting a polynucleotide of interest into the genome of a cell. Also provided are nucleic acid constructs and cells having an altered target site or altered polynucleotide of interest produced by the methods described herein.

### CROSS-REFERENCE TO RELATED APPLICATIONS

This application is a 371 national stage entry of International Application No. PCT/US17/155717 filed 27 Feb. 2017, which claims the benefit of U.S. Provisional Application No. 62/306904, filed Mar. 11, 2016, which is incorporated herein in its entirety by reference.

### FIELD

The disclosure relates to the field of plant molecular biology, in particular, to compositions for novel guided Cas9 endonuclease systems and compositions and methods for altering the genome of a cell.

### REFERENCE TO SEQUENCE LISTING SUBMITTED ELECTRONICALLY

The official copy of the sequence listing is submitted electronically via EFS-Web as an ASCII formatted sequence listing with a file named 20170223_BB2398PCT_ST25.txt created on Feb. 23, 2017 and having a size of 605 kilobytes and is filed concurrently with the specification. The sequence listing contained in this ASCII formatted document is part of the specification and is herein incorporated by reference in its entirety.

### BACKGROUND

Recombinant DNA technology has made it possible to modify (edit) specific endogenous chromosomal sequences and/or insert DNA sequences at targeted genomic locations thus altering the organism's phenotype. Site-specific integration techniques, which employ site-specific recombination systems, as well as other types of recombination technologies, have been used to generate targeted insertions of genes of interest in a variety of organism. Genome-editing techniques such as designer zinc finger nucleases (ZFNs) or transcription activator-like effector nucleases (TALENs), or homing meganucleases, are available for producing targeted genome perturbations, but these systems tends to have a low specificity and employ designed nucleases that need to be redesigned for each target site, which renders them costly and time-consuming to prepare.

Although several approaches have been developed to target a specific site for modification in the genome of an organism, there still remains a need for new genome engineering technologies that are affordable, easy to set up, scalable, and amenable to targeting multiple positions within the genome of an organism.

### BRIEF SUMMARY

Compositions and methods are provided for novel Cas9 systems and elements comprising such systems, including, but not limiting to, novel guide polynucleotide/Cas9 endonucleases complexes, single guide RNAs, guide RNA elements, and Cas9 endonucleases.

In one embodiment of the disclosure, the guide RNA is a single guide RNA capable of forming a guide RNA/Cas9 endonuclease complex, wherein said guide RNA/Cas9 endonuclease complex can recognize, bind to, and optionally nick or cleave a target sequence, wherein said single guide RNA is selected from the group consisting of SEQ ID NOs: 185-207, a functional fragment of SEQ ID NOs: 185-207, and a functional variant of SEQ ID NOs: 185-207.

In one embodiment of the disclosure, the guide RNA is single guide RNA capable of forming a guide RNA/Cas9 endonuclease complex, wherein said guide RNA/Cas9 endonuclease complex can recognize, bind to, and optionally nick or cleave a target sequence, wherein said single guide RNA comprises a chimeric non-naturally occurring crRNA linked to a tracrRNA, wherein said tracrRNA comprises a nucleotide sequence selected from the group consisting of SEQ ID NOs: 139-184, a functional fragment of SEQ ID NOs: 139-184, and a functional variant of SEQ ID NOs: 139-184.

In one embodiment of the disclosure, the guide RNA is a single guide RNA capable of forming a guide RNA/Cas9 endonuclease complex, wherein said guide RNA/Cas9 endonuclease complex can recognize, bind to, and optionally nick or cleave a target sequence, wherein said single guide RNA comprises a chimeric non-naturally occurring crRNA linked to a tracrRNA, wherein said chimeric non-naturally occurring crRNA comprises a nucleotide sequence selected from the group consisting of SEQ ID NOs: 116-138, a functional fragment of SEQ ID NOs: 116-138, and a functional variant of SEQ ID NOs: 116-138.

The guide RNA can also be a dual molecule comprising a chimeric non-naturally occurring crRNA linked to a tracrRNA, wherein said chimeric non-naturally occurring crRNA comprises a nucleotide sequence selected from the group consisting of SEQ ID NOs: 116-138, a functional fragment of SEQ ID NOs: 116-138, and a functional variant of SEQ ID NOs: 116-138, and/or wherein said tracrRNA comprises a nucleotide sequence selected from the group consisting of SEQ ID NOs: 139-184, a functional fragment of SEQ ID NOs: 139-184, and a functional variant of SEQ ID NOs: 139-184.

In one embodiment of the disclosure, the guide RNA/Cas9 endonuclease complex is a guide RNA/Cas9 endonuclease complex comprising at least one guide RNA and a Cas9 endonuclease, wherein said Cas9 endonuclease is encoded by a DNA sequence selected from the group consisting of SEQ ID NOs: 24-46, wherein said guide RNA/Cas9 endonuclease complex is capable of recognizing, binding to, and optionally nicking or cleaving all or part of a target sequence.

In one embodiment of the disclosure, the method is a method for modifying a target site in the genome of a cell, the method comprising introducing into said cell at least one guide RNA and at least one Cas9 endonuclease selected from the group consisting of SEQ ID NOs: 47-69, a functional fragment of SEQ ID NOs: 47-69, and a functional variant of SEQ ID NOs: 47-69, wherein said guide RNA and Cas9 endonuclease can form a complex that is capable of recognizing, binding to, and optionally nicking or cleaving all or part of said target site. The method can further comprise identifying at least one cell that has a modification at said target, wherein the modification at said target site is selected from the group consisting of (i) a replacement of at least one nucleotide, (ii) a deletion of at least one nucleotide, (iii) an insertion of at least one nucleotide, and (iv) any combination of (i)-(iii).

In one embodiment of the disclosure, the method is a method for editing a nucleotide sequence in the genome of a cell, the method comprising introducing into said cell a polynucleotide modification template, at least one guide RNA and at least one Cas9 endonuclease selected from the group consisting of SEQ ID NOs: 47-69, a functional fragment of SEQ ID NOs: 47-69, and a functional variant of SEQ ID NOs: 47-69, wherein said polynucleotide modification template comprises at least one nucleotide modification of said nucleotide sequence, wherein said guide RNA and Cas9 endonuclease can form a complex that is capable of recognizing, binding to, and optionally nicking or cleaving all or part of said target site.

In one embodiment of the disclosure, the method is a method for modifying a target site in the genome of a cell, the method comprising introducing into said cell at least one guide RNA, at least one donor DNA, and at least one Cas9 endonuclease selected from the group consisting of SEQ ID NOs: 47-69, a functional fragment of SEQ ID NOs: 47-69, and a functional variant of SEQ ID NOs: 47-69, wherein said at least one guide RNA and at least one Cas9 endonuclease can form a complex that is capable of recognizing, binding to, and optionally nicking or cleaving all or part of said target site, wherein said donor DNA comprises a polynucleotide of interest.

Also provided are nucleic acid constructs, plants, plant cells, explants, seeds and grain having an altered target site or altered polynucleotide of interest produced by the methods described herein. Additional embodiments of the methods and compositions of the present disclosure are shown herein.

### BRIEF DESCRIPTION OF THE DRAWINGS AND THE SEQUENCE LISTING

The disclosure can be more fully understood from the following detailed description and the accompanying drawings and Sequence Listing, which form a part of this application. The sequence descriptions and sequence listing attached hereto comply with the rules governing nucleotide and amino acid sequence disclosures in patent applications as set forth in 37 C.F.R. §§ 1.821-1.825. The sequence descriptions contain the three letter codes for amino acids as defined in 37 C.F.R. §§ 1.821-1.825, which are incorporated herein by reference.

### FIGURES

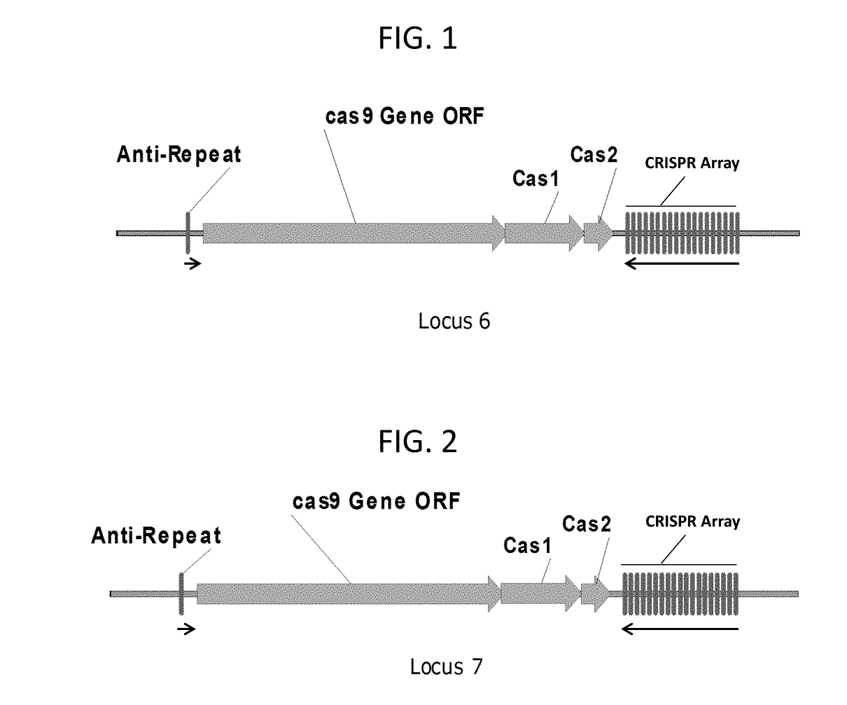

FIG. 1 shows a diagram of a genomic DNA region from Bacillus cereus representing a CRISPR-Cas locus (referred to as Locus 6) described herein. Arrows indicate the transcriptional directional of the anti-repeat and the CRISPR repeats within the CRISPR array (CRISPR array). Cas9 Gene ORF refers to the open reading frame of the Cas9 endonuclease. Cas1 and Cas2 refer to the open reading frame of the Cas1 and Cas2 protein, respectively.

FIG. 2 shows a diagram of a genomic DNA region from Brevibacillus laterosporus representing a CRISPR-Cas locus (referred to as Locus 7) described herein.

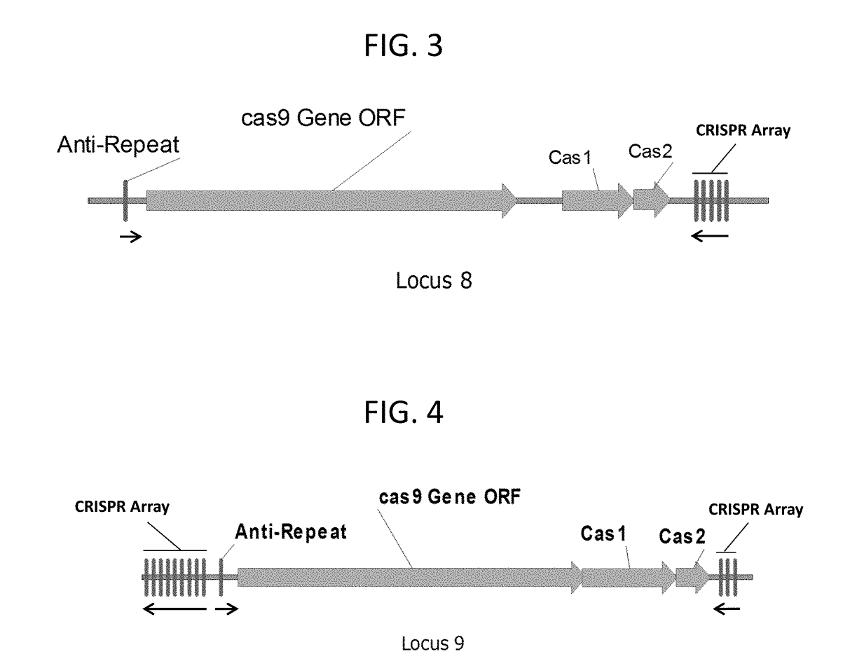

FIG. 3 shows a diagram of a genomic DNA region from Bacillus species representing a CRISPR-Cas locus (referred to as Locus 8) described herein.

FIG. 4 shows a diagram of a genomic DNA region from Bacillus cereus representing a CRISPR-Cas locus (referred to as Locus 9) described herein.

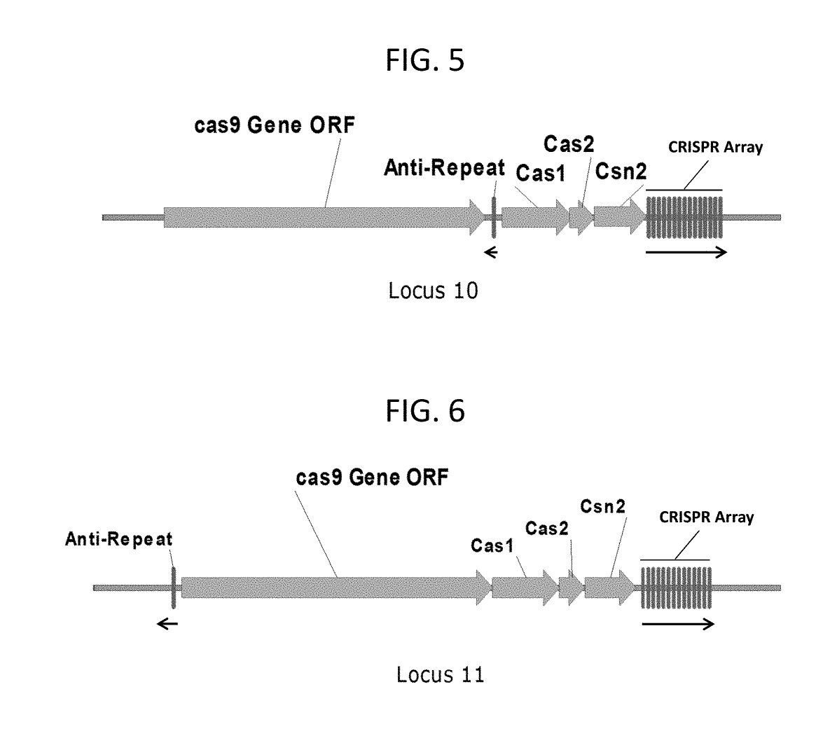

FIG. 5 shows a diagram of a genomic DNA region from Lactobacillus fermentum representing a CRISPR-Cas locus (referred to as Locus 10) described herein. Arrows indicate the transcriptional directional of the anti-repeat and the CRISPR repeat within the CRISPR array. Cas9 Gene ORF refers to the open reading frame of the Cas9 endonuclease. Cas1, Cas2, Csn2 refer to the open reading frame of the Cas1, Cas2 and Csn2 protein, respectively.

FIG. 6 shows a diagram of a genomic DNA region from Enterococcus faecalis representing a CRISPR-Cas locus (referred to as Locus 11) described herein.

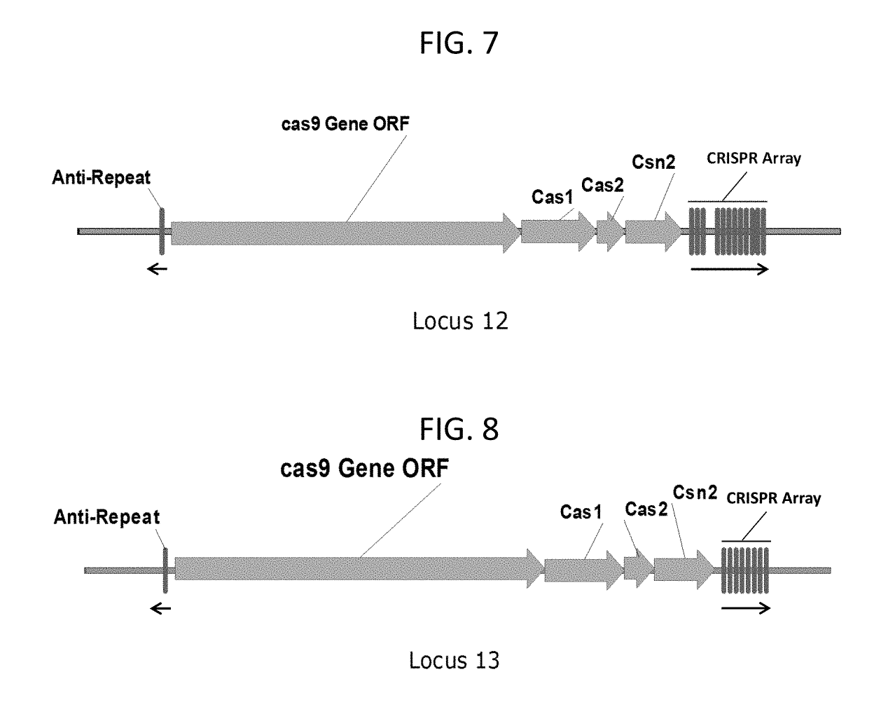

FIG. 7 shows a diagram of a genomic DNA region from Bacillus cereus representing a CRISPR-Cas locus (referred to as Locus 12) described herein.

FIG. 8 shows a diagram of a genomic DNA region from Enterococcus faecalis representing a CRISPR-Cas locus (referred to as Locus 13) described herein.

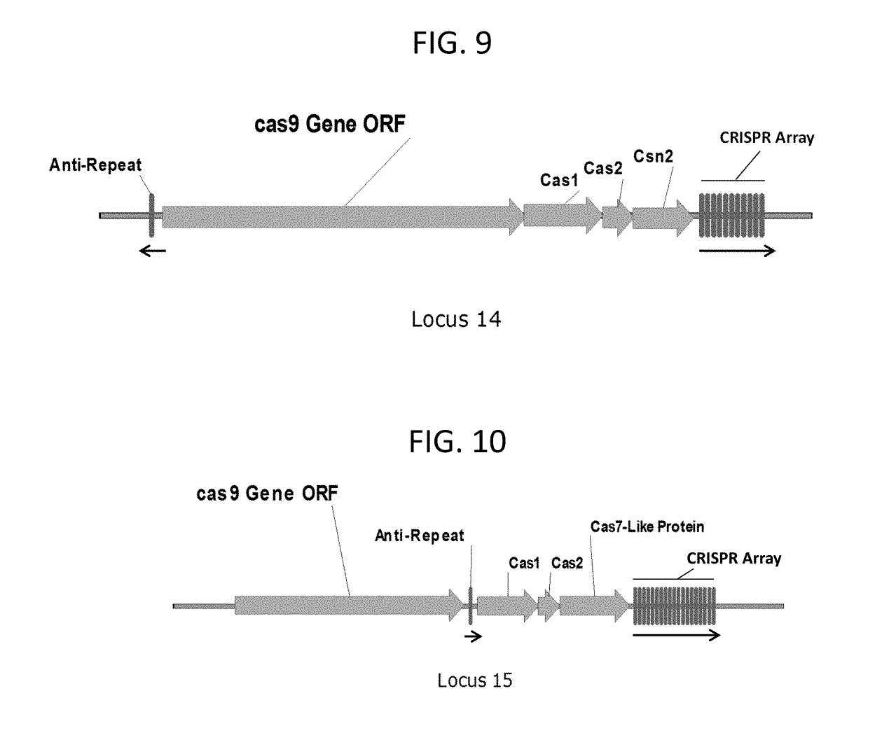

FIG. 9 shows a diagram of a genomic DNA region from an unknown organism representing a CRISPR-Cas locus (referred to as Locus 14) described herein.

FIG. 10 shows a diagram of a genomic DNA region from Enterococcus faecalis representing a CRISPR-Cas locus (referred to as Locus 15) described herein. Arrows indicate the transcriptional directional of the anti-repeat and the CRISPR repeats within the CRISPR array (CRISPR array). Cas9 Gene ORF refers to the open reading frame of the Cas9 endonuclease. Cas1, Cas2 and Cas7-Like protein refer to the open reading frame of the Cas1, Cas2 and Cas7-Like protein, respectively.

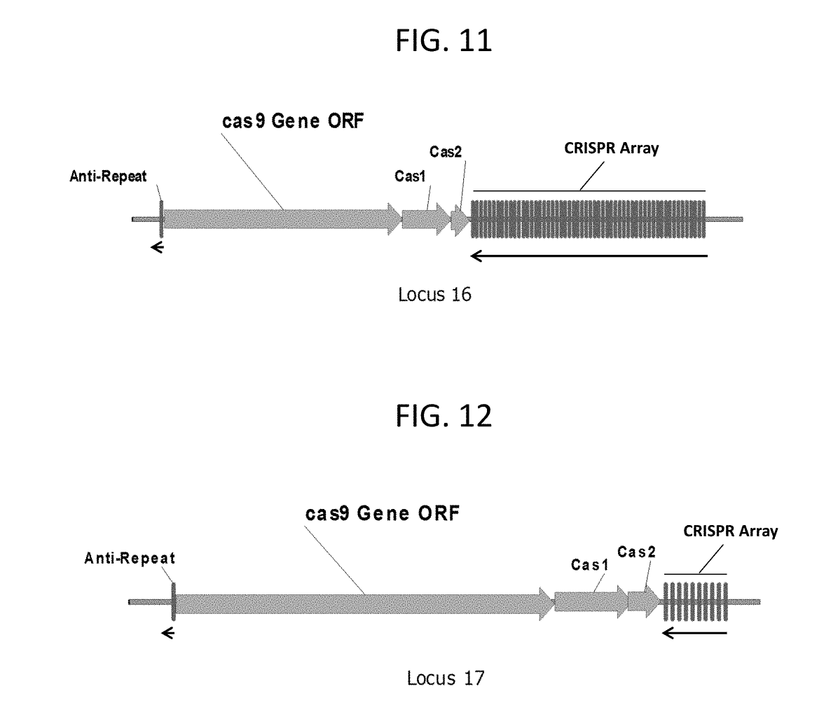

FIG. 11 shows a diagram of a genomic DNA region from metagenomic material representing a CRISPR-Cas locus (referred to as Locus 16) described herein.

FIG. 12 shows a diagram of a genomic DNA region from Chryseobacterium species representing a CRISPR-Cas locus (referred to as Locus 17) described herein.

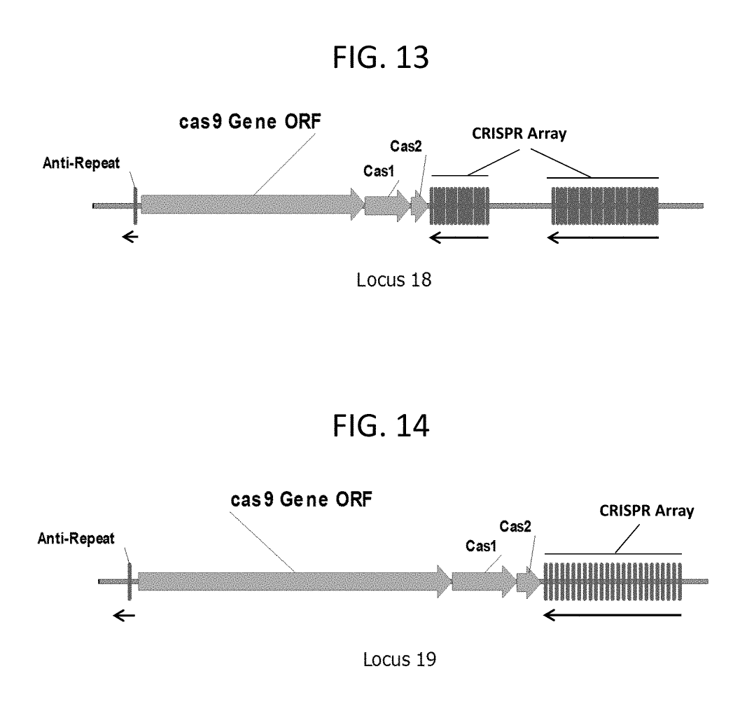

FIG. 13 shows a diagram of a genomic DNA region from metagenomic material representing a CRISPR-Cas locus (referred to as Locus 18) described herein.

FIG. 14 shows a diagram of a genomic DNA region from metagenomic material representing a CRISPR-Cas locus (referred to as Locus 19) described herein.

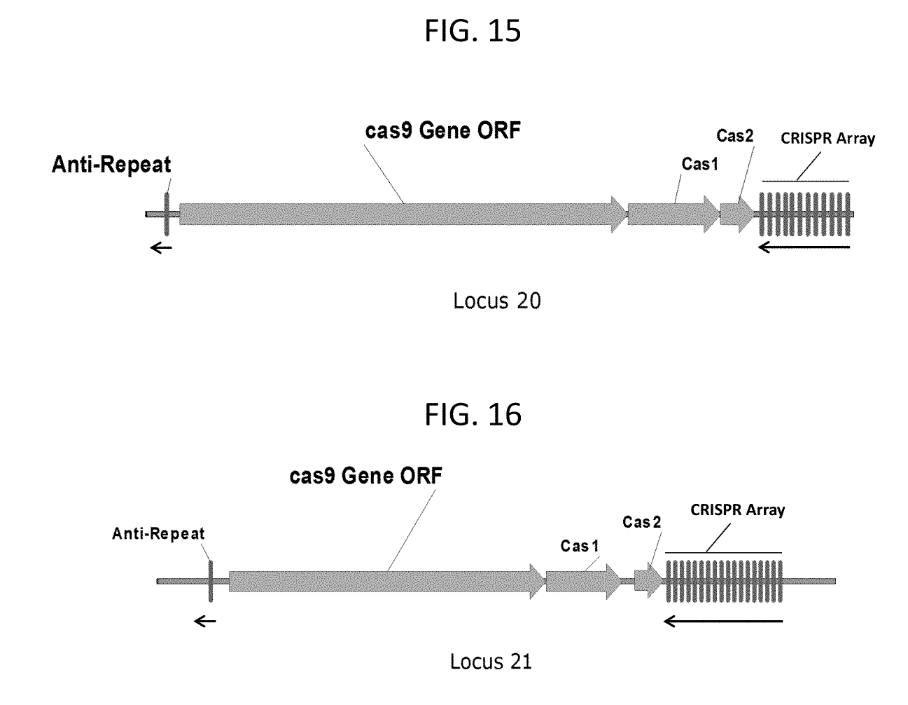

FIG. 15 shows a diagram of a genomic DNA region from an unknown organism representing a CRISPR-Cas locus (referred to as Locus 20) described herein.

FIG. 16 shows a diagram of a genomic DNA region from metagenomic material representing a CRISPR-Cas locus (referred to as Locus 21) described herein.

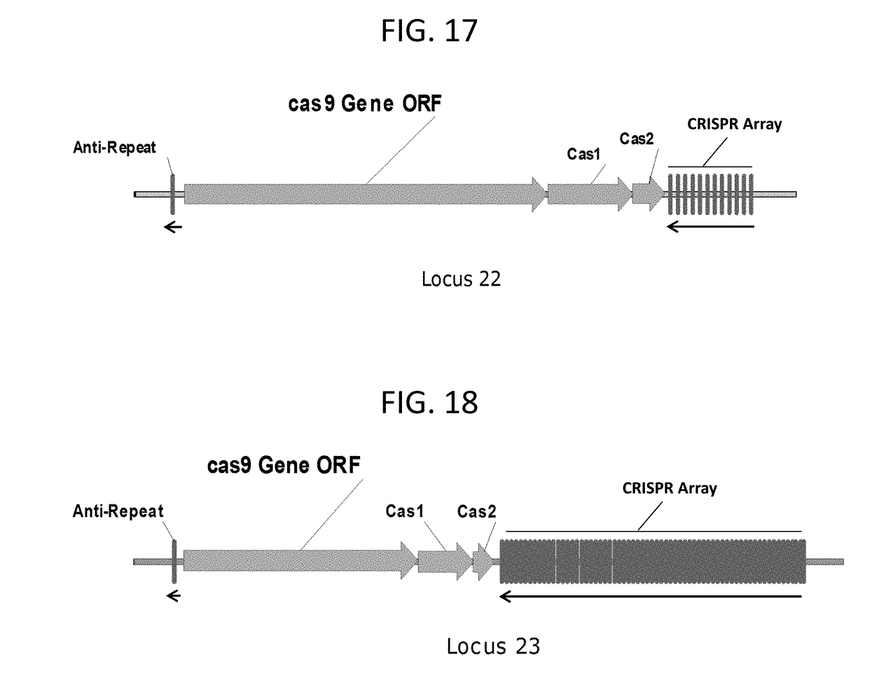

FIG. 17 shows a diagram of a genomic DNA region from metagenomic material representing a CRISPR-Cas locus (referred to as Locus 22) described herein.

FIG. 18 shows a diagram of a genomic DNA region from metagenomic material representing a CRISPR-Cas locus (referred to as Locus 23) described herein.

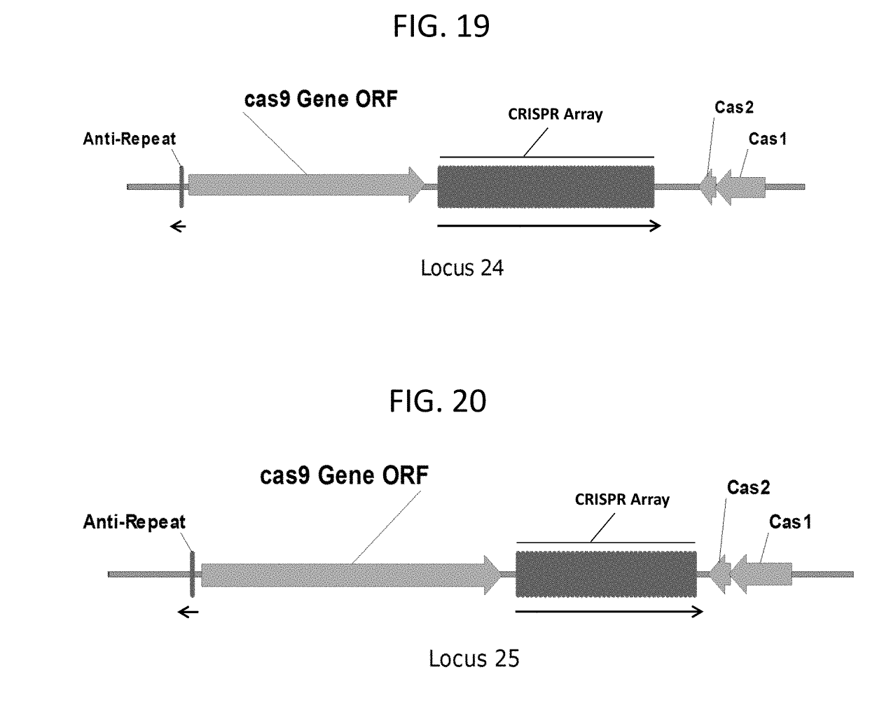

FIG. 19 shows a diagram of a genomic DNA region from metagenomic material representing a CRISPR-Cas locus (referred to as Locus 24) described herein.

FIG. 20 shows a diagram of a genomic DNA region from metagenomic material representing a CRISPR-Cas locus (referred to as Locus 25) described herein.

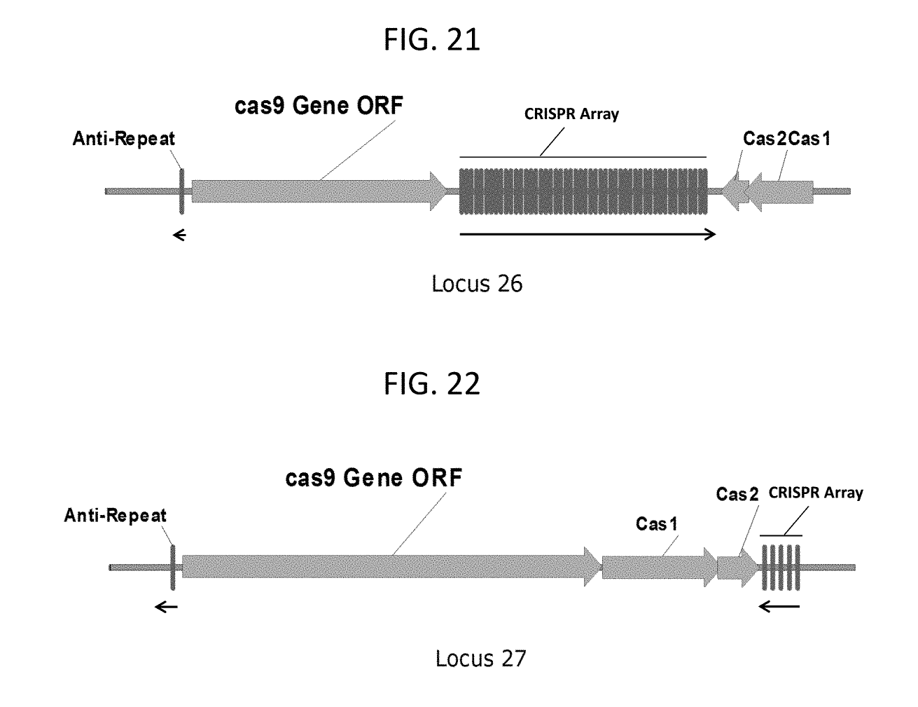

FIG. 21 shows a diagram of a genomic DNA region from metagenomic material representing a CRISPR-Cas locus (referred to as Locus 26) described herein.

FIG. 22 shows a diagram of a genomic DNA region from metagenomic material representing a CRISPR-Cas locus (referred to as Locus 27) described herein.

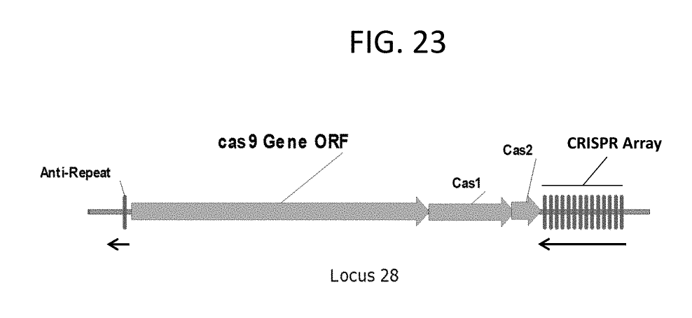

FIG. 23 shows a diagram of a genomic DNA region from metagenomic material representing a CRISPR-Cas locus (referred to as Locus 28) described herein.

### SEQUENCES

TABLE 1
Summary of Nucleic Acid and Protein SEQ ID Numbers

| | Nucleic acid | Protein |
|---|---|---|
| Description | SEQ ID NO: | SEQ ID NO: |
| Locus 6 | 1 | |
| Locus 7 | 2 | |
| Locus 8 | 3 | |
| Locus 9 | 4 | |
| Locus 10 | 5 | |
| Locus 11 | 6 | |
| Locus 12 | 7 | |
| Locus 13 | 8 | |
| Locus 14 | 9 | |
| Locus 15 | 10 | |
| Locus 16 | 11 | |
| Locus 17 | 12 | |
| Locus 18 | 13 | |
| Locus 19 | 14 | |
| Locus 20 | 15 | |
| Locus 21 | 16 | |
| Locus 22 | 17 | |
| Locus 23 | 18 | |
| Locus 24 | 19 | |
| Locus 25 | 20 | |
| Locus 26 | 21 | |
| Locus 27 | 22 | |
| Locus 28 | 23 | |
| Cas9 endonuclease from Locus 6 | 24-46 | 47-69 |
| to Locus 28, respectively | | |
| CRISPR repeat consensus from | 70-92 | |
| Locus 6 to Locus 28, respectively | | |
| Anti-Repeat from Locus 6 to | 93-115 | |
| Locus 28, respectively | | |
| sgRNA repeat region | 116-138 | |
| (Locus 6 to Locus 28, respectively) | | |
| sgRNA anti Repeat region | 139-161 | |
| (Locus 6 to Locus 28, respectively) | | |
| 3′ tracrRNA in gRNA | 162-184 | |
| (Locus 6 to Locus 28, respectively) | | |
| sgRNAs | 185-207 | |

### DETAILED DESCRIPTION

Compositions are provided for novel Cas9 systems and elements comprising such systems, including, but not limiting to, novel guide polynucleotide/Cas9 endonucleases complexes, single guide RNAs, guide RNA elements, and Cas9 endonucleases. The present disclosure further includes compositions and methods for genome modification of a target sequence in the genome of a cell, for gene editing, and for inserting a polynucleotide of interest into the genome of a cell.

CRISPR (clustered regularly interspaced short palindromic repeats) loci refers to certain genetic loci encoding factors of DNA cleavage systems, for example, used by bacterial and archaeal cells to destroy foreign DNA (Horvath and Barrangou, 2010, Science 327:167-170). A CRISPR locus can consist of a CRISPR array, comprising short direct repeats (CRISPR repeats) separated by short variable DNA sequences (called ‘spacers’), which can be flanked by diverse Cas (CRISPR-associated) genes. Multiple CRISPR-Cas systems have been described including Class 1 systems, with multisubunit effector complexes, and Class 2 systems, with single protein effectors (such as but not limiting to Cas9, Cpf1, C2c1,C2c2, C2c3). (Zetsche et al., 2015, Cell 163, 1-13; Shmakov et al., 2015, Molecular_Cell 60, 1-13; Makarova et al. 2015, Nature Reviews Microbiology Vol. 13:1-15, WO 2013/176772 A1 published on Nov. 23, 2013 and incorporated by its entirety by reference herein). CRISPR systems belong to different classes, with different repeat patterns, sets of genes, and species ranges.

The type II CRISPR/Cas system from bacteria employs a crRNA (CRISPR RNA) and tracrRNA (trans-activating CRISPR RNA) to guide a Cas9 endonuclease (encoded by a cas9 gene) to its DNA target. The crRNA contains a spacer region complementary to one strand of the double strand DNA target and a region that base pairs with the tracrRNA (trans-activating CRISPR RNA) forming a RNA duplex that directs the Cas9 endonuclease to cleave the DNA target. Spacers are acquired through a not fully understood process involving Cas1 and Cas2 proteins. All type II CRISPR-Cas loci contain cas1 and cas2 genes in addition to the cas9 gene (Makarova et al. 2015, Nature Reviews Microbiology Vol. 13:1-15). Type II CRISPR-Cas loci can encode a tracrRNA, which is partially complementary to the repeats within the respective CRISPR array, and can comprise other proteins such as Csn1 and Csn2. The presence of cas9 in the vicinity of cas1 and cas2 genes is the hallmark of type II loci (Makarova et al. 2015, Nature Reviews Microbiology Vol. 13:1-15).

The number of CRISPR-associated genes at a given CRISPR locus can vary between species (Haft et al., 2005, Computational Biology, PLoS Comput Biol 1(6): e60. doi:10.1371/journal.pcbi.0010060; Makarova et al. 2015, Nature Reviews Microbiology Vol. 13:1-15; WO 2013/176772 A1 published on Nov. 23, 2013 and incorporated by its entirety by reference herein).

“Cas9” (formerly referred to as Cas5, Csn1, or Csx12) herein refers to a Cas (CRISPR-associated) endonuclease that when in complex with a suitable polynucleotide component (such as crNucleotide and a tracrNucleotide, or a single guide polynucleotide) is capable of recognizing, binding to, and optionally nicking or cleaving all or part of a DNA target sequence. A Cas9 protein comprises a HNH domain and a RuvC nuclease domain, each of which can cleave a single DNA strand at a target sequence (the concerted action of both domains leads to DNA double-strand cleavage, whereas activity of one domain leads to a nick). In general, the RuvC domain comprises subdomains I, II and III, where domain I is located near the N-terminus of Cas9 and subdomains II and III are located in the middle of the protein, flanking the HNH domain (Hsu et al, Cell 157:1262-1278). Cas9 endonucleases are typically derived from a type II CRISPR system, which includes a DNA cleavage system utilizing a Cas9 endonuclease in complex with at least one polynucleotide component. For example, a Cas9 can be in complex with a CRISPR RNA (crRNA) and a trans-activating CRISPR RNA (tracrRNA). In another example, a Cas9 can be in complex with a single guide RNA (Makarova et al. 2015, Nature Reviews Microbiology Vol. 13:1-15).

The term “cas9 gene” herein refers to a gene encoding a Cas9 endonuclease.

As used herein, the terms “guide polynucleotide/Cas9 endonuclease complex”, “guide polynucleotide/Cas9 endonuclease system”, “guide polynucleotide/Cas9 complex”, “guide polynucleotide/Cas9 system”, “Polynucleotide-guided endonuclease”, “PGEN”, “guided Cas system” are used interchangeably herein and refer to at least one guide polynucleotide and at least one Cas9 endonuclease that are capable of forming a complex, wherein said guide polynucleotide/Cas9 endonuclease complex can direct the Cas9 endonuclease to a DNA target site, enabling the Cas9 endonuclease to recognize, bind to, and optionally nick or cleave (introduce a single or double strand break) the DNA target site. A guide polynucleotide/Cas9 endonuclease complex herein can comprise Cas9 protein(s) and suitable polynucleotide component(s) of any of the known CRISPR systems (Horvath and Barrangou, 2010, Science 327:167-170; Makarova et al. 2015, Nature Reviews Microbiology Vol. 13:1-15). A Cas9 endonuclease unwinds the DNA duplex at the target sequence and optionally cleaves at least one DNA strand, as mediated by recognition of the target sequence by a polynucleotide (such as, but not limited to, a crRNA or guide RNA) that is in complex with the Cas9 protein. Such recognition and cutting of a target sequence by a Cas9 endonuclease typically occurs if the correct protospacer-adjacent motif (PAM) is located at or adjacent to the 3′ end of the DNA target sequence. Alternatively, a Cas9 protein herein may lack DNA cleavage or nicking activity, but can still specifically bind to a DNA target sequence when complexed with a suitable RNA component. (See also U.S. Patent Application US 2015-0082478 A1, published on Mar. 19, 2015 and US 2015-0059010 A1, published on Feb. 26, 2015, both are incorporated in their entirety by reference herein).

A guide polynucleotide/Cas9 endonuclease complex can cleave one or both strands of a DNA target sequence. A guide polynucleotide/Cas9 endonuclease complex that can cleave both strands of a DNA target sequence typically comprises a Cas9 protein that has all of its endonuclease domains in a functional state (e.g., wild type endonuclease domains or variants thereof retaining some or all activity in each endonuclease domain). Thus, a wild type Cas9 protein, or a variant thereof retaining some or all activity in each endonuclease domain of the Cas9 protein, is a suitable example of a Cas9 endonuclease that can cleave both strands of a DNA target sequence. A Cas9 protein comprising functional RuvC and HNH nuclease domains is an example of a Cas9 protein that can cleave both strands of a DNA target sequence. A guide polynucleotide/Cas9 endonuclease complex that can cleave one strand of a DNA target sequence can be characterized herein as having nickase activity (e.g., partial cleaving capability). A Cas9 nickase typically comprises one functional endonuclease domain that allows the Cas9 to cleave only one strand (i.e., make a nick) of a DNA target sequence. For example, a Cas9 nickase may comprise (i) a mutant, dysfunctional RuvC domain and (ii) a functional HNH domain (e.g., wild type HNH domain). As another example, a Cas9 nickase may comprise (i) a functional RuvC domain (e.g., wild type RuvC domain) and (ii) a mutant, dysfunctional HNH domain. Non-limiting examples of Cas9 nickases suitable for use herein are disclosed in U.S. Patent Appl. Publ. No. 2014/0189896, which is incorporated herein by reference.

A pair of Cas9 nickases can be used to increase the specificity of DNA targeting. In general, this can be done by providing two Cas9 nickases that, by virtue of being associated with RNA components with different guide sequences, target and nick nearby DNA sequences on opposite strands in the region for desired targeting. Such nearby cleavage of each DNA strand creates a double strand break (i.e., a DSB with single-stranded overhangs), which is then recognized as a substrate for non-homologous-end-joining, NHEJ (prone to imperfect repair leading to mutations) or homologous recombination, HR. Each nick can be at least about 5, 10, 15, 20, 30, 40, 50, 60, 70, 80, 90, or 100 (or any integer between 5 and 100) bases apart from each other, for example. One or two Cas9 nickase proteins herein can be used in a Cas9 nickase pair. For example, a Cas9 nickase with a mutant RuvC domain, but functioning HNH domain (i.e., Cas9 HNH+/RuvC-), could be used (e.g., Streptococcus pyogenes Cas9 HNH+/RuvC-). Each Cas9 nickase (e.g., Cas9 HNH+/RuvC-) would be directed to specific DNA sites nearby each other (up to 100 base pairs apart) by using suitable RNA components herein with guide RNA sequences targeting each nickase to each specific DNA site.

A Cas9 protein can be part of a fusion protein comprising one or more heterologous protein domains (e.g., 1, 2, 3, or more domains in addition to the Cas9 protein). Such a fusion protein may comprise any additional protein sequence, and optionally a linker sequence between any two domains, such as between Cas9 and a first heterologous domain. Examples of protein domains that may be fused to a Cas9 protein herein include, without limitation, epitope tags (e.g., histidine [His], V5, FLAG, influenza hemagglutinin [HA], myc, VSV-G, thioredoxin [Trx]), reporters (e.g., glutathione-5-transferase [GST], horseradish peroxidase [HRP], chloramphenicol acetyltransferase [CAT], beta-galactosidase, beta-glucuronidase [GUS], luciferase, green fluorescent protein [GFP], HcRed, DsRed, cyan fluorescent protein [CFP], yellow fluorescent protein [YFP], blue fluorescent protein [BFP]), and domains having one or more of the following activities: methylase activity, demethylase activity, transcription activation activity (e.g., VP16 or VP64), transcription repression activity, transcription release factor activity, histone modification activity, RNA cleavage activity and nucleic acid binding activity. A Cas9 protein can also be in fusion with a protein that binds DNA molecules or other molecules, such as maltose binding protein (MBP), S-tag, Lex A DNA binding domain (DBD), GAL4A DNA binding domain, and herpes simplex virus (HSV) VP16.

A guide polynucleotide/Cas9 endonuclease complex in certain embodiments can bind to a DNA target site sequence, but does not cleave any strand at the target site sequence. Such a complex may comprise a Cas9 protein in which all of its nuclease domains are mutant, dysfunctional. For example, a Cas9 protein herein that can bind to a DNA target site sequence, but does not cleave any strand at the target site sequence, may comprise both a mutant, dysfunctional RuvC domain and a mutant, dysfunctional HNH domain. A Cas9 protein herein that binds, but does not cleave, a target DNA sequence can be used to modulate gene expression, for example, in which case the Cas protein could be fused with a transcription factor (or portion thereof) (e.g., a repressor or activator, such as any of those disclosed herein).

The Cas9 endonuclease gene herein can be a plant, microbial, animal or mammalian codon optimized Cas9 endonuclease gene. The Cas9 endonuclease gene can be operably linked to a SV40 nuclear targeting signal upstream of the Cas codon region and a bipartite VirD2 nuclear localization signal (Tinland et al. (1992) Proc. Natl. Acad. Sci. USA 89:7442-6) downstream of the Cas9 codon region.

Cas9 endonucleases are typically derived from a type II CRISPR system, which includes a DNA cleavage system utilizing a Cas9 endonuclease in complex with at least one polynucleotide component. For example, a Cas9 can be in complex with a CRISPR RNA (crRNA) and a trans-activating CRISPR RNA (tracrRNA). In another example, a Cas9 can be in complex with a single guide RNA (Makarova et al. 2015, Nature Reviews Microbiology Vol. 13:1-15).

In one embodiment of the disclosure, the composition comprises at least one Cas9 endonuclease selected from the group consisting of SEQ ID NOs: 47-69, a functional fragment of SEQ ID NOs: 47-69, and a functional variant of SEQ ID NOs: 47-69.

In one embodiment of the disclosure, the composition comprises at least one recombinant DNA (such as a vector) encoding the Cas9 endonuclease selected from the group consisting of SEQ ID NOs: 47-69, a functional fragment of SEQ ID NOs: 47-69, and a functional variant of SEQ ID NOs: 47-69 (such as a recombinant DNA comprising the DNA sequences form SEQ ID NO: 24-46, a functional fragment of SEQ ID NOs: 24-46, and a functional variant of SEQ ID NOs: 24-46), or mRNA encoding Cas9 endonuclease selected from the group consisting of SEQ ID NOs: 47-69, a functional fragment of SEQ ID NOs: 47-69, and a functional variant of SEQ ID NOs: 47-69. The Cas9 endonuclease selected from the group consisting of SEQ ID NOs: 47-69, a functional fragment of SEQ ID NOs: 47-69, and a functional variant of SEQ ID NOs: 47-69 can form a (Ribonucleotide Protein—RNP) complex with at least one guide RNA, wherein said complex is capable of recognizing, binding to, and optionally nicking or cleaving all or part of a target site.

Recombinant DNA expressing the Cas9 endonucleases described herein (including, variants, functional fragments thereof, plant -, microbe -,or mammalian-codon optimized Cas9 endonuclease) can be stably integrated into the genome of an organism. For example, plants can be produced that comprise a cas9 gene stably integrated in the plant's genome. Plants expressing a stably integrated Cas endonuclease can be exposed to at least one guide RNA and/or a polynucleotide modification templates and/or donor DNAs to enable genome modifications such as gene knockout, gene editing or DNA insertions.

The terms “functional fragment”, “fragment that is functionally equivalent” and “functionally equivalent fragment” of a Cas9 endonuclease are used interchangeably herein, and refer to a portion or subsequence of the Cas9 endonuclease sequence of the present disclosure in which the ability to recognize, bind to, and optionally nick or cleave (introduce a single or double strand break in) the target site is retained.

Functional fragments of a Cas9 endonuclease of the present disclosure include proteins comprising at least one domain selected from the group consisting of a guide polynucleotide binding domain (an amino acid domain that can bind to or hybridize to a guide RNA), a crRNA binding domain (an amino acid domain that can bind to or hybridize to a crRNA), a tracrRNA binding domain (an amino acid domain that can bind to or hybridize to a tracrRNA), a DNA binding domain (an amino acid domain that can bind to DNA target sequence), a DNA cleavage domain (such as an HNH or RuvC domain) and any combination thereof.

Functional fragments of Cas9 endonucleases of the present disclosure include fragments comprising 50-100, 100-200, 100-300, 100-400, 100-500, 100-600, 100-700, 100-800, 100-900, 100-1000, 200-300, 200-400, 200-500, 200-600, 200-700, 200-800, 200-900, 200-1000, 300-400, 300-500, 300-600, 300-700, 300-800, 300-900, 300-1000, 400-500, 400-600, 400-700, 400-800, 400-900, 400-1000, 500-600, 500-700, 500-800, 500-900, 500-1000, 600-700, 600-800, 600-900, 600-1000, 700-800, 700-900, 700-1000, 800-900, 800-1000, or 900-1000 amino acids of a reference Cas9 protein, such as the reference Cas9 endonucleases of the present disclosure of SEQ ID NOs:46-69.

Functional fragments of the Cas9 endonucleases of the present disclosure include a protein comprising one or more protein domains of the Cas9 endonuclease of SEQ ID NOs: 46-69, wherein said protein retains specific binding activity, and optionally endonucleolytic activity, towards a target DNA when associated with a polynucleotide component.

The terms “functional variant”, “Variant that is functionally equivalent” and “functionally equivalent variant” of a Cas9 endonuclease are used interchangeably herein, and refer to a variant of the Cas9 endonuclease of the present disclosure in which the ability to recognize, bind to, and optionally nick or cleave (introduce a single or double strand break in) the target site is retained.

A functional variant of a Cas9 protein sequence may be used, but should have specific binding activity, and optionally endonucleolytic activity, toward DNA when associated with a polynucleotide component herein. Such a functional variant of Cas9 may comprise an amino acid sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of the reference Cas9, such as the reference Cas9 endonucleases described herein, including the Cas9 endonucleases of SEQ ID NOs: 47-69. Such a variant Cas9 protein can have specific binding activity, and optionally cleavage or nicking activity, toward DNA when associated with an RNA component herein. Cas9 variants include Cas9 endonuclease proteins wherein the HNH domain and/or the RuvC domain contains at least one amino acid change (e.g., deletion, insertion, or substitution). In some embodiments, the amino acid variation resulting in at least about 80%, 81%, 82%, 83%, 84%, 85%, 86%, 87%, 88%, 89%, 90%, 91%, 92%, 93%, 94%, 95%, 96%, 97%, 98%, or 99% identity to the amino acid sequence of the reference Cas9 protein, is located inside the HNH domain, or inside the RuvC domain, or inside both the HNH and RuvC domain. In some embodiments, the amino acid variation resulting in at least about 80%, 81%, 82%, 83%, 84%, 85%, 86%, 87%, 88%, 89%, 90%, 91%, 92%, 93%, 94%, 95%, 96%, 97%, 98%, or 99% identity to the amino acid sequence of the reference Cas9 protein, is located outside of the HNH domain, or outside the RuvC domain, or outside both the HNH and RuvC domain.

Multiple functional domains and conserved elements were determined for each of the novel Cas9 endonuclease protein of the present disclosure (see Example 2, Tables 13-14). The novel Cas9 endonucleases of the present disclosure comprised an HNH domain, an RuvC domain that included three subdomains (RuvC-I, Ruvc-II and RuvC-II), a Bridge-Helix domain a PAM interacting domain and DNA/RNA recognition regions including REC1 and REC1′. The REC1 binds to repeat:anti-repeat RNA duplex of the guide RNA while REC1′ mainly interacts with targetDNA:guide RNA hybrid duplex. The REC2 domain is a conserved element.

In some aspects the RuvC-I domain of a Cas9 endonuclease can be 40, 41, 42, 43, 44, 45, 46, 47, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 or 80 amino acids in length. The RuvC-I domain can be located next to anyone of the amino acid domains selected from the group consisting of HNH, RuvC-I, RuvC-II, RuvC-III, REC1, REC1′, REC-2, Bridge Helix (BH) and PAM interacting (PI) domain. Tables 13-14 described herein the location of the RuvC-I domain of each of the Cas9 endonucleases of the present disclosure and based on this information one can design novel Cas9 endonucleases comprising any one of the RuvC-I domain selected from the group consisting of the RuvC-I domain of Cas-Locus-6, the RuvC-I domain of Cas-Locus 7, the RuvC-I domain of Cas-locus-8, the RuvC-I domain of Cas-Locus-9, the RuvC-I domain of Cas-locus-10, the RuvC-I domain of Cas-Locus-11, Cas-Locus-12, the RuvC-I domain of Cas-Locus 13, the RuvC-I domain of Cas-locus-14, the RuvC-I domain of Cas-Locus-15, the RuvC-I domain of Cas-locus-16, the RuvC-I domain of Cas-Locus-17, Cas-Locus-18, the RuvC-I domain of Cas-Locus 19, the RuvC-I domain of Cas-locus-20, the RuvC-I domain of Cas-Locus-21, the RuvC-I domain of Cas-locus-22, the RuvC-I domain of Cas-Locus-23, the RuvC-I domain of Cas-locus-24, the RuvC-I domain of Cas-Locus-25, the RuvC-I domain of Cas-locus-26, the RuvC-I domain of Cas-Locus-27, the RuvC-I domain of Cas-locus-28, a function fragment thereof, and a functional variant thereof. (A functional fragment or functional variant of a RuvC-I domain is a fragment or variant in which the ability to function as a RuvC-I domain is retained).

In some aspects the Bridge-Helix (BH) domain of a Cas9 endonuclease can be 35, 36, 37, 38, 39, 40, 41, 42, 43, 44, 45, 46 or 47 amino acids in length. The BH domain can be located next to anyone of the amino acid domains selected from the group consisting of HNH, RuvC-I, RuvC-II, RuvC-III, REC1, REC1′, REC-2, Bridge Helix (BH) and PAM interacting (PI) domain. Tables 13-14 described herein the location of the BH domain of each of the Cas9 endonucleases of the present disclosure and based on this information one can design novel Cas9 endonucleases comprising any one of the BH domain selected from the group consisting of the BH domain of Cas-Locus-6, the BH domain of Cas-Locus 7, the BH domain of Cas-locus-8, the BH domain of Cas-Locus-9, the BH domain of Cas-locus-10, the BH domain of Cas-Locus-11, Cas-Locus-12, the BH domain of Cas-Locus 13, the BH domain of Cas-locus-14, the BH domain of Cas-Locus-15, the BH domain of Cas-locus-16, the BH domain of Cas-Locus-17, Cas-Locus-18, the BH domain of Cas-Locus 19, the BH domain of Cas-locus-20, the BH domain of Cas-Locus-21, the BH domain of Cas-locus-22, the BH domain of Cas-Locus-23, the BH domain of Cas-locus-24, the BH domain of Cas-Locus-25, the BH domain of Cas-locus-26, the BH domain of Cas-Locus-27, the BH domain of Cas-locus-28, a function fragment thereof, and a functional variant thereof. (A functional fragment or functional variant of a BH domain is a fragment or variant in which the ability to function as a BH domain is retained).

In some aspects the REC1 domain of a Cas9 endonuclease can be 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304 or 305 amino acids in length. The REC1 domain can be located next to anyone of the amino acid domains selected from the group consisting of HNH, RuvC-I, RuvC-II, RuvC-III, REC1, REC1′, REC-2, Bridge Helix (BH) and PAM interacting (PI) domain. Tables 13-14 described herein the location of the REC1 domain of each of the Cas9 endonucleases of the present disclosure and based on this information one can design novel Cas9 endonucleases comprising any one of the REC1 domain selected from the group consisting of the REC1 domain of Cas-Locus-6, the REC1 domain of Cas-Locus 7, the REC1 domain of Cas-locus-8, the REC1 domain of Cas-Locus-9, the REC1 domain of Cas-locus-10, the REC1 domain of Cas-Locus-11, Cas-Locus-12, the REC1 domain of Cas-Locus 13, the REC1 domain of Cas-locus-14, the REC1 domain of Cas-Locus-15, the REC1 domain of Cas-locus-16, the REC1 domain of Cas-Locus-17, Cas-Locus-18, the REC1 domain of Cas-Locus 19, the REC1 domain of Cas-locus-20, the REC1 domain of Cas-Locus-21, the REC1 domain of Cas-locus-22, the REC1 domain of Cas-Locus-23, the REC1 domain of Cas-locus-24, the REC1 domain of Cas-Locus-25, the REC1 domain of Cas-locus-26, the REC1 domain of Cas-Locus-27, the REC1 domain of Cas-locus-28, a function fragment thereof, and a functional variant thereof. (A functional fragment or functional variant of a REC1 domain is a fragment or variant in which the ability to function as a REC1 domain is retained).

In some aspects the REC2 domain of a Cas9 endonuclease can be 130, 131, 132, 133, 134, 135, 136, 137, 138, 139, 140, 141, 142, 143, 144, 145, 146, 147, 148, 149, 150, 151, 152, 153, 154, 155, or 156 amino acids in length. The REC2 domain can be located next to anyone of the amino acid domains selected from the group consisting of HNH, RuvC-I, RuvC-II, RuvC-III, REC1, REC1′, REC-2, Bridge Helix (BH) and PAM interacting (PI) domain. Tables 13-14 described herein the location of the REC2 domain of each of the Cas9 endonucleases of the present disclosure and based on this information one can design novel Cas9 endonucleases comprising any one of the REC2 domain selected from the group consisting of the REC2 domain of Cas-Locus-6, the REC2 domain of Cas-Locus 7, the REC2 domain of Cas-locus-8, the REC2 domain of Cas-Locus-9, the REC2 domain of Cas-locus-10, the REC2 domain of Cas-Locus-11, Cas-Locus-12, the REC2 domain of Cas-Locus 13, the REC2 domain of Cas-locus-14, the REC2 domain of Cas-Locus-15, the REC2 domain of Cas-locus-16, the REC2 domain of Cas-Locus-17, Cas-Locus-18, the REC2 domain of Cas-Locus 19, the REC2 domain of Cas-locus-20, the REC2 domain of Cas-Locus-21, the REC2 domain of Cas-locus-22, the REC2 domain of Cas-Locus-23, the REC2 domain of Cas-locus-24, the REC2 domain of Cas-Locus-25, the REC2 domain of Cas-locus-26, the REC2 domain of Cas-Locus-27, the REC2 domain of Cas-locus-28, a function fragment thereof, and a functional variant thereof. (A functional fragment or functional variant of a REC2 domain is a fragment or variant in which the ability to function as a REC2 domain is retained).

In some aspects the REC1′ domain of a Cas9 endonuclease can be 213, 214, 215, 216, 217, 218, 219, 220, 221,222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 360, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 405, 406, 407, 408, 409, 410, 411, 412, 413, 414, 415, 416 or 417 amino acids in length. The REC1′ domain can be located next to anyone of the amino acid domains selected from the group consisting of HNH, RuvC-I, RuvC-II, RuvC-III, REC1, REC1′, REC-2, Bridge Helix (BH) and PAM interacting (PI) domain. Tables 13-14 described herein the location of the REC1′ domain of each of the Cas9 endonucleases of the present disclosure and based on this information one can design novel Cas9 endonucleases comprising any one of the REC1′ domain selected from the group consisting of the REC1′ domain of Cas-Locus-6, the REC1′ domain of Cas-Locus 7, the REC1′ domain of Cas-locus-8, the REC1′ domain of Cas-Locus-9, the REC1′ domain of Cas-locus-10, the REC1′ domain of Cas-Locus-11, Cas-Locus-12, the REC1′ domain of Cas-Locus 13, the REC1′ domain of Cas-locus-14, the REC1′ domain of Cas-Locus-15, the REC1′ domain of Cas-locus-16, the REC1′ domain of Cas-Locus-17, Cas-Locus-18, the REC1′ domain of Cas-Locus 19, the REC1′ domain of Cas-locus-20, the REC1′ domain of Cas-Locus-21, the REC1′ domain of Cas-locus-22, the REC1′ domain of Cas-Locus-23, the REC1′ domain of Cas-locus-24, the REC1′ domain of Cas-Locus-25, the REC1′ domain of Cas-locus-26, the REC1′ domain of Cas-Locus-27, the REC1′ domain of Cas-locus-28, a function fragment thereof, and a functional variant thereof. (A functional fragment or functional variant of a REC1′ domain is a fragment or variant in which the ability to function as a REC1′ domain is retained).

In some aspects the RuvC-II domain of a Cas9 endonuclease can be 45, 46, 47, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137,138,139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244 or 245 amino acids in length. The RuvC-II domain can be located next to anyone of the amino acid domains selected from the group consisting of HNH, RuvC-I, RuvC-II, RuvC-III, REC1, REC1′, REC-2, Bridge Helix (BH) and PAM interacting (PI) domain. Tables 13-14 described herein the location of the RuvC-II domain of each of the Cas9 endonucleases of the present disclosure and based on this information one can design novel Cas9 endonucleases comprising any one of the RuvC-II domain selected from the group consisting of the RuvC-II domain of Cas-Locus-6, the RuvC-II domain of Cas-Locus 7, the RuvC-II domain of Cas-locus-8, the RuvC-II domain of Cas-Locus-9, the RuvC-II domain of Cas-locus-10, the RuvC-II domain of Cas-Locus-11, Cas-Locus-12, the RuvC-II domain of Cas-Locus 13, the RuvC-II domain of Cas-locus-14, the RuvC-II domain of Cas-Locus-15, the RuvC-II domain of Cas-locus-16, the RuvC-II domain of Cas-Locus-17, Cas-Locus-18, the RuvC-II domain of Cas-Locus 19, the RuvC-II domain of Cas-locus-20, the RuvC-II domain of Cas-Locus-21, the RuvC-II domain of Cas-locus-22, the RuvC-II domain of Cas-Locus-23, the RuvC-II domain of Cas-locus-24, the RuvC-II domain of Cas-Locus-25, the RuvC-II domain of Cas-locus-26, the RuvC-II domain of Cas-Locus-27, the RuvC-II domain of Cas-locus-28, a function fragment thereof, and a functional variant thereof. (A functional fragment or functional variant of a RuvC-II domain is a fragment or variant in which the ability to function as a RuvC-II domain is retained).

In some aspects the HNH domain of a Cas9 endonuclease can be 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216 or 217 amino acids in length. The HNH domain can be located next to anyone of the amino acid domains selected from the group consisting of HNH, RuvC-I, RuvC-II, RuvC-III, REC1, REC1′, REC-2, Bridge Helix (BH) and PAM interacting (PI) domain. Tables 13-14 described herein the location of the HNH domain of each of the Cas9 endonucleases of the present disclosure and based on this information one can design novel Cas9 endonucleases comprising any one of the HNH domain selected from the group consisting of the HNH domain of Cas-Locus-6, the HNH domain of Cas-Locus 7, the HNH domain of Cas-locus-8, the HNH domain of Cas-Locus-9, the HNH domain of Cas-locus-10, the HNH domain of Cas-Locus-11, Cas-Locus-12, the HNH domain of Cas-Locus 13, the HNH domain of Cas-locus-14, the HNH domain of Cas-Locus-15, the HNH domain of Cas-locus-16, the HNH domain of Cas-Locus-17, Cas-Locus-18, the HNH domain of Cas-Locus 19, the HNH domain of Cas-locus-20, the HNH domain of Cas-Locus-21, the HNH domain of Cas-locus-22, the HNH domain of Cas-Locus-23, the HNH domain of Cas-locus-24, the HNH domain of Cas-Locus-25, the HNH domain of Cas-locus-26, the HNH domain of Cas-Locus-27, the HNH domain of Cas-locus-28, a function fragment thereof, and a functional variant thereof. (A functional fragment or functional variant of a HNH domain is a fragment or variant in which the ability to function as a HNH domain is retained).

In some aspects the RuvC-III domain of a Cas9 endonuclease can be 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257 or 258 amino acids in length. The RuvC-III domain can be located next to anyone of the amino acid domains selected from the group consisting of HNH, RuvC-I, RuvC-II, RuvC-III, REC1, REC1′, REC-2, Bridge Helix (BH) and PAM interacting (PI) domain. Tables 13-14 described herein the location of the RuvC-III domain of each of the Cas9 endonucleases of the present disclosure and based on this information one can design novel Cas9 endonucleases comprising any one of the RuvC-III domain selected from the group consisting of the RuvC-III domain of Cas-Locus-6, the RuvC-III domain of Cas-Locus 7, the RuvC-III domain of Cas-locus-8, the RuvC-III domain of Cas-Locus-9, the RuvC-III domain of Cas-locus-10, the RuvC-III domain of Cas-Locus-11, Cas-Locus-12, the RuvC-III domain of Cas-Locus 13, the RuvC-III domain of Cas-locus-14, the RuvC-III domain of Cas-Locus-15, the RuvC-III domain of Cas-locus-16, the RuvC-III domain of Cas-Locus-17, Cas-Locus-18, the RuvC-III domain of Cas-Locus 19, the RuvC-III domain of Cas-locus-20, the RuvC-III domain of Cas-Locus-21, the RuvC-III domain of Cas-locus-22, the RuvC-III domain of Cas-Locus-23, the RuvC-III domain of Cas-locus-24, the RuvC-III domain of Cas-Locus-25, the RuvC-III domain of Cas-locus-26, the RuvC-III domain of Cas-Locus-27, the RuvC-III domain of Cas-locus-28, a function fragment thereof, and a functional variant thereof.

In some aspects the PI domain of a Cas9 endonuclease can be 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321,322, 323, 324, 325, 326, 327, 328, 329, 330, 331, 332, 333, 334, 335, 336, 337, 338, 339, 340, or 341 amino acids in length. The PI domain can be located next to anyone of the amino acid domains selected from the group consisting of HNH, RuvC-I, RuvC-II, RuvC-III, REC1, REC1′, REC-2, Bridge Helix (BH) and PAM interacting (PI) domain. Tables 13-14 described herein the location of the PI domain of each of the Cas9 endonucleases of the present disclosure and based on this information one can design novel Cas9 endonucleases comprising any one of the PI domain selected from the group consisting of the PI domain of Cas-Locus-6, the PI domain of Cas-Locus 7, the PI domain of Cas-locus-8, the PI domain of Cas-Locus-9, the PI domain of Cas-locus-10, the PI domain of Cas-Locus-11, Cas-Locus-12, the PI domain of Cas-Locus 13, the PI domain of Cas-locus-14, the PI domain of Cas-Locus-15, the PI domain of Cas-locus-16, the PI domain of Cas-Locus-17, Cas-Locus-18, the PI domain of Cas-Locus 19, the PI domain of Cas-locus-20, the PI domain of Cas-Locus-21, the PI domain of Cas-locus-22, the PI domain of Cas-Locus-23, the PI domain of Cas-locus-24, the PI domain of Cas-Locus-25, the PI domain of Cas-locus-26, the PI domain of Cas-Locus-27, the PI domain of Cas-locus-28, a function fragment thereof, and a functional variant thereof. (A functional fragment or functional variant of a PI domain is a fragment or variant in which the ability to function as a PI domain is retained).

Cas9 endonuclease functional fragments and Cas9 endonuclease variants can be obtained via methods such as site-directed mutagenesis and synthetic construction

Methods for determining if fragments and/or variants of a Cas9 endonuclease of the present disclosure are functional include methods that measure the endonuclease activity of the fragment or variant when in complex with a suitable polynucleotide. Methods that measure Cas9 endonuclease activity are well known in the art such as, but not limiting to, PCT/US13/39011, filed May 1, 2013, PCT/US16/32073 filed May 12, 2016, PCT/US16/32028 filed May 12, 2016, incorporated by reference herein). Methods for measuring Cas9 endonuclease activity include methods that measure the mutation frequency at a target site after a double strand break has occurred (see also, Example 3).

Methods for measuring Cas9 endonuclease activity include methods that measure the mutation frequency at a target site after a double strand break has occurred (see also, Example 3). Methods for measuring if a functional fragment or functional variant of a Cas9 endonuclease of the present disclosure can make a double strand break include the following method: The cellular repair of chromosomal double-strand breaks (DSBs) induced by CRISPR-Cas9 in plant cells results in the production of insertion or deletion (indel) mutagenesis (Svitashev et al. et al. (2015)). This outcome can be used to detect and monitor the production of DSBs generated by functional fragments of functional variant of the Cas9 endonucleases of the present disclosure (see also Karvelis et al. (2015). Briefly, appropriate CRISPR-Cas9 maize genomic DNA target sites can be selected, a guide RNA transcriptional cassette (recombinant DNA that expresses a guide RNA) and a DNA recombinant construct expressing the Cas9 endonuclease of the present disclosure (or a functional fragment of the Cas9 endonuclease of the present disclosure, or a functional variant of the Cas9 endonuclease variant of the present disclosure endonuclease (such as an expression cassette described in Example 2) can be constructed and can be co-delivered by biolistic transformation into Hi-Type II 10-day-old immature maize embryos (IMEs) in the presence of BBM and WUS2 genes as described in Svitashev et al. (2015). A visual marker DNA expression cassette encoding a yellow fluorescent protein can also be co-delivered with the guide RNA transcriptional cassette and the Cas9 endonuclease expression cassette (recombinant DNA construct) to aid in the selection of evenly transformed IMEs. After 2 days, the 20-30 most evenly transformed IMEs can be harvested based on their fluorescence. Total genomic DNA is extracted and the DNA region surrounding the intended target site is PCR amplified with Phusion® HighFidelity PCR Master Mix (New England Biolabs, M0531 L) adding on the sequences necessary for amplicon-specific barcodes and Illumnia sequencing and deep sequenced. The resulting reads are then examined for the presence of mutations at the expected site of cleavage by comparison to control experiments where the guide RNA transcriptional cassette was omitted from the transformation. If mutations are observed at the intended target sites when using a fragment or variant of the Cas9 endonuclease of the present disclosure, in complex with a suitable guide polynucleotide, the fragments or variants are functional.

Methods for measuring if a functional fragment of functional variant of a Cas9 endonuclease of the present disclosure can make a single strand break (also referred to as a nick; hence acts as a nickase) in the double stranded DNA target site include the following method: The cellular repair of chromosomal single-strand breaks (SSBs) in a double-stranded DNA target may be typically repaired seamlessly in plant cells such as maize. Therefore to examine a functional Cas9 fragment or functional variant of a Cas9 for nicking activity, two chromosomal DNA target sites in close proximity (0-200 bp), each targeting a different strand (sense and anti-sense DNA strands) of the double-stranded DNA, can be targeted. If SSB activity is present, the SSB activity from both target sites will result in a DNA double-strand break (DSB) that will result in the production of insertion or deletion (indel) mutagenesis in maize cells. This outcome can then be used to detect and monitor the activity of the Cas9 nickase similar to that described in Karvelis et al. (2015). Briefly, appropriate CRISPR-Cas9 maize genomic DNA target sites are selected, guide RNA transcription cassettes and functional fragment Cas9 nicking expression cassettes are constructed and co-delivered by biolistic transformation into Hi-Type II 10-day-old immature maize embryos (IMEs) in the presence of BBM and WUS2 genes as described in Svitashev et al. (2015). Since particle gun transformation can be highly variable, a visual marker DNA expression cassette encoding a yellow fluorescent protein can also be co-delivered to aid in the selection of evenly transformed IMEs [immature maize embryos]. After 2 days, the 20-30 most evenly transformed IMEs are harvested based on their fluorescence, total genomic DNA extracted, the region surrounding the intended target site PCR amplified with Phusion® HighFidelity PCR Master Mix (New England Biolabs, M0531 L) adding on the sequences necessary for amplicon-specific barcodes and Illumnia sequencing and deep sequenced. The resulting reads are then examined for the presence of mutations at the expected site of cleavage by comparison to control experiments where the small RNA transcriptional cassette was omitted from the transformation.

Methods for measuring if a functional fragment of functional variant of a Cas9 endonuclease of the present disclosure can bind to the intended DNA target site include the following method: The binding of a maize chromosomal DNA target site does not result in either a single-stranded break (SSB) or a double-stranded break (DSB) in the double-stranded DNA target site. Therefore to examine a functional Cas9 fragment for binding activity in maize cells, another nuclease domain (e.g. Fokl) may be attached to the functional Cas9 fragment with binding activity. If binding activity is present, the added nuclease domain may be used to produce a DSB that will result in the production of insertion or deletion (indel) mutagenesis in maize cells. This outcome may then be used to detect and monitor the binding activity of a Cas9 similar to that described in Karvelis et al. (2015). Briefly, appropriate CRISPR-Cas9 maize genomic DNA target sites can be selected, guide RNA transcription cassettes and functional fragment Cas9 binding and nuclease attached expression cassettes can be constructed and co-delivered by biolistic transformation into Hi-Type II 10-day-old immature maize embryos (IMEs) in the presence of BBM and WUS2 genes as described in Svitashev et al. (2015). A visual marker DNA expression cassette encoding a yellow fluorescent protein can also be co-delivered to aid in the selection of evenly transformed IMEs [immature maize embryos]. After 2 days, the 20-30 most evenly transformed IMEs can be harvested based on their fluorescence, total genomic DNA extracted, the region surrounding the intended target site PCR amplified with Phusion® HighFidelity PCR Master Mix (New England Biolabs, M0531 L) adding on the sequences necessary for amplicon-specific barcodes and Illumnia sequencing and deep sequenced. The resulting reads can then be examined for the presence of mutations at the expected site of cleavage by comparison to control experiments where the small RNA transcriptional cassette was omitted from the transformation.

Alternatively, the binding activity of maize chromosomal DNA target sites can be monitored by the transcriptional induction or repression of a gene. This can be accomplished by attaching a transcriptional activation or repression domain to the functional Cas9 binding fragment and targeting it to the promoter region of a gene and binding monitored through an increase in accumulation of the gene transcript or protein. The gene targeted for either activation or repression can be any naturally occurring maize gene or engineered gene (e.g. a gene encoded a red fluorescent protein) introduced into the maize genome by methods known in the art (e.g. particle gun or agrobacterium transformation).

The Cas9 endonuclease can comprise a modified form of the Cas9 polypeptide. The modified form of the Cas9 polypeptide can include an amino acid change (e.g., deletion, insertion, or substitution) that reduces the naturally-occurring nuclease activity of the Cas9 protein. For example, in some instances, the modified form of the Cas9 protein has less than 50%, less than 40%, less than 30%, less than 20%, less than 10%, less than 5%, or less than 1% of the nuclease activity of the corresponding wild-type Cas9 polypeptide (US patent application US20140068797 A1, published on Mar. 6, 2014). In some cases, the modified form of the Cas9 polypeptide has no substantial nuclease activity and is referred to as catalytically “inactivated Cas9” or “deactivated cas9 (dCas9).” Catalytically inactivated Cas9 variants include Cas9 variants that contain mutations in the HNH and RuvC nuclease domains. These catalytically inactivated Cas9 variants are capable of interacting with sgRNA and binding to the target site in vivo but cannot cleave either strand of the target DNA.

A catalytically inactive Cas9 can be fused to a heterologous sequence (US patent application US20140068797 A1, published on Mar. 6, 2014). Suitable fusion partners include, but are not limited to, a polypeptide that provides an activity that indirectly increases transcription by acting directly on the target DNA or on a polypeptide (e.g., a histone or other DNA-binding protein) associated with the target DNA. Additional suitable fusion partners include, but are not limited to, a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, or demyristoylation activity. Further suitable fusion partners include, but are not limited to, a polypeptide that directly provides for increased transcription of the target nucleic acid (e.g., a transcription activator or a fragment thereof, a protein or fragment thereof that recruits a transcription activator, a small molecule/drug-responsive transcription regulator, etc.). A catalytically inactive Cas9 can also be fused to a Fokl nuclease to generate double strand breaks (Guilinger et al. Nature biotechnology, volume 32, number 6, June 2014).

A Cas9 protein herein can comprise a heterologous nuclear localization sequence (NLS). A heterologous NLS amino acid sequence herein may be of sufficient strength to drive accumulation of a Cas9 protein in a detectable amount in the nucleus of a yeast cell herein, for example. An NLS may comprise one (monopartite) or more (e.g., bipartite) short sequences (e.g., 2 to 20 residues) of basic, positively charged residues (e.g., lysine and/or arginine), and can be located anywhere in a Cas9 amino acid sequence but such that it is exposed on the protein surface. An NLS may be operably linked to the N-terminus or C-terminus of a Cas9 protein herein, for example. Two or more NLS sequences can be linked to a Cas9 protein, for example, such as on both the N- and C-termini of a Cas9 protein. The Cas9 endonuclease gene can be operably linked to a SV40 nuclear targeting signal upstream of the Cas9 codon region and a bipartite VirD2 nuclear localization signal (Tinland et al. (1992) Proc. Natl. Acad. Sci. USA 89:7442-6) downstream of the Cas9 codon region. Non-limiting examples of suitable NLS sequences herein include those disclosed in U.S. Pat. No. 7,309,576, which is incorporated herein by reference.

Cas9 endonucleases described herein can be used for targeted genome editing (via simplex and multiplex double-strand breaks and nicks) and targeted genome regulation (via tethering of epigenetic effector domains to either the Cas9 protein or guide polynucleotide (sgRNA or combination of crRNA+tracrRNA). A Cas9 endonuclease can also be engineered to function as an RNA-guided recombinase, and via RNA tethers could serve as a scaffold for the assembly of multiprotein and nucleic acid complexes (Mali et al., 2013, Nature Methods Vol. 10: 957-963).

The Cas9 protein, or functional fragment thereof, for use in the disclosed methods, can be isolated from a recombinant source where the genetically modified host cell (e.g. an insect cell or a yeast cell or human-derived cell line) is modified to express the nucleic acid sequence encoding the Cas9 protein. Alternatively, the Cas9 protein can be produced using cell free protein expression systems or be synthetically produced.

The term “plant-optimized Cas9 endonuclease” herein refers to a Cas9 protein encoded by a nucleotide sequence that has been optimized for expression in a plant cell or plant, and optionally for increased expression in a plant. A “plant-optimized nucleotide sequence encoding a Cas9 endonuclease”, “plant-optimized construct encoding a Cas9 endonuclease” and a “plant-optimized polynucleotide encoding a Cas9” are used interchangeably herein and refer to a nucleotide sequence encoding an Cas9 protein, or a variant or functional fragment thereof, that has been optimized for expression in a plant cell or plant. A plant comprising a plant-optimized Cas9 endonuclease includes a plant comprising the nucleotide sequence encoding for the Cas9 sequence and/or a plant comprising the Cas9 endonuclease protein. In one aspect, the plant-optimized Cas9 endonuclease nucleotide sequence is a maize-optimized, rice-optimized, wheat-optimized or soybean-optimized Cas9 endonuclease.

A plant-optimized nucleotide sequence, such as a plant-optimized Cas9 endonuclease DNA sequence, can be synthesized by modifying a nucleotide sequence using one or more plant-preferred codons for improved expression. See, for example, Campbell and Gowri (1990) Plant Physiol. 92:1-11 for a discussion of host-preferred codon usage.

The term “mammalian-optimized Cas9 endonuclease sequence” herein refers to a nucleotide sequence encoding a Cas9 endonuclease that has been optimized for expression in mammalian cells, particularly for increased expression in mammalian cells.

The Cas9 endonuclease described herein can be introduced into a cell by any method known in the art, for example, but not limited to transient introduction methods (such as Agrobacterium-mediated transformation, or particle mediated delivery such as biolistic particle bombardment), transfection, microinjection, and/or topical application or indirectly via recombination constructs. The Cas9 endonuclease can be introduced as a protein or as a guided polynucleotide complex (ribonucleotide complex, RNP complex) directly to a cell or indirectly via recombination constructs. The endonuclease can be introduced into a cell transiently or can be incorporated into the genome of the host cell using any method known in the art. Uptake of the endonuclease and/or the guided polynucleotide into the cell can be facilitated with a Cell Penetrating Peptide (CPP) as described in U.S. application 62/075,999, filed Nov. 6, 2014.

As used herein, the term “guide polynucleotide”, relates to a polynucleotide sequence that can form a complex with a Cas9 endonuclease and enables the Cas9 endonuclease to recognize, bind to, and optionally cleave a DNA target site. The guide polynucleotide can be a single molecule or a double molecule. The guide polynucleotide sequence can be a RNA sequence, a DNA sequence, or a combination thereof (a RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also referred to as a “guide RNA” or “gRNA” (See also U.S. Patent Application US 2015-0082478 A1, published on Mar. 19, 2015 and US 2015-0059010 A1, published on Feb. 26, 2015, both are hereby incorporated in its entirety by reference).

The guide polynucleotide can be a double molecule (also referred to as duplex guide polynucleotide) comprising a crNucleotide sequence and a tracrNucleotide sequence. The crNucleotide includes a first nucleotide sequence domain (referred to as Variable Targeting domain or VT domain) that can hybridize to a nucleotide sequence in a target DNA and a second nucleotide sequence (also referred to as a tracr mate sequence) that is part of a Cas endonuclease recognition (CER) domain. The tracr mate sequence can hybridized to a tracrNucleotide along a region of complementarity and together form the Cas endonuclease recognition domain or CER domain. The CER domain is capable of interacting with a Cas9 endonuclease polypeptide. The crNucleotide and the tracrNucleotide of the duplex guide polynucleotide can be RNA, DNA, and/or RNA-DNA-combination sequences. In some embodiments, the crNucleotide molecule of the duplex guide polynucleotide is referred to as “crDNA” (when composed of a contiguous stretch of DNA nucleotides) or “crRNA” (when composed of a contiguous stretch of RNA nucleotides), or “crDNA-RNA” (when composed of a combination of DNA and RNA nucleotides). The crNucleotide can comprise a fragment of the crRNA naturally occurring in Bacteria and Archaea. The size of the fragment of the crRNA naturally occurring in Bacteria and Archaea that can be present in a crNucleotide disclosed herein can range from, but is not limited to, 2, 3, 4, 5, 6, 7, 8, 9,10, 11, 12, 13, 14, 15, 16, 17, 18, 19, 20 or more nucleotides. In some embodiments the tracrNucleotide is referred to as “tracrRNA” (when composed of a contiguous stretch of RNA nucleotides) or “tracrDNA” (when composed of a contiguous stretch of DNA nucleotides) or “tracrDNA-RNA” (when composed of a combination of DNA and RNA nucleotides. In one embodiment, the RNA that guides the RNA/ Cas9 endonuclease complex is a duplexed RNA comprising a duplex crRNA-tracrRNA.

The tracrRNA (trans-activating CRISPR RNA, Deltcheva et al., Nature 471:602-607) contains, in the 5′-to-3′ direction, (i) a sequence that anneals with the repeat region of CRISPR type II crRNA (described herein as the anti-repeat region, such as but not limiting to SEQ ID NOs: 138-161), and (ii) a stem loop-containing portion (described herein as a 3′ tracrRNA such as but not limiting to SEQ ID NOs: 162-184)

The tracrRNA component of the single or duplex guide RNA for the Cas9 endonuclease systems described herein can comprise an anti-repeat fragment (including any one of SEQ ID NOs: 139-161) and a 3′ tracrRNA component (including any one of SEQ ID NO: 162-184). For example the tracrRNA can comprise SEQ ID NOs: 139 and 162, or SEQ ID NOs: 140 and 163, or SEQ ID NOs: 141 and 164, or SEQ ID NOs: 142 and 165, or SEQ ID NOs: 143 and 166, or SEQ ID NOs: 144 and 167, or SEQ ID NOs: 145 and 168, or SEQ ID NOs: 146 and 169, or SEQ ID NOs: 147 and 170, or SEQ ID NOs: 148 and 171, or SEQ ID NOs: 149 and 172, or SEQ ID NOs: 150 and 173, or SEQ ID NOs: 151 and 174, or SEQ ID NOs: 152 and 175, or SEQ ID NOs: 153 and 176, or SEQ ID NOs: 154 and 177, or SEQ ID NOs: 155 and 178, or SEQ ID NOs: 156 and 179, or SEQ ID NOs: 157 and 180, or SEQ ID NOs: 158 and 181, or SEQ ID NOs: 159 and 182, or SEQ ID NOs: 160 and 183, or SEQ ID NOs: 161 and 184.

The duplex guide polynucleotide can form a complex with a Cas9 endonuclease, wherein said guide polynucleotide/Cas9 endonuclease complex (also referred to as a guide polynucleotide/Cas9 endonuclease system) can direct the Cas9 endonuclease to a genomic target site, enabling the Cas9 endonuclease to recognize, bind to, and optionally nick or cleave (introduce a single or double strand break) into the target site. (See also U.S. Patent Application US 2015-0082478 A1, published on Mar. 19, 2015 and US 2015-0059010 A1, published on Feb. 26, 2015, both are hereby incorporated in its entirety by reference.)

A chimeric non-naturally occurring crRNA includes a crRNA that comprises regions that are not found together in nature (i.e., they are heterologous with each other. For example, a crRNA comprising a first nucleotide sequence domain (referred to as Variable Targeting domain or VT domain) that can hybridize to a nucleotide sequence in a target DNA, linked to a second nucleotide sequence (also referred to as a tracr mate sequence) such that the first and second sequence are not found linked together in nature. In one such example, a chimeric non-naturally occurring crRNA includes a VT domain that is capable of recognizing (or binding to) a target sequence in a eukaryotic genome.

The guide polynucleotide can also be a single molecule (also referred to as single guide polynucleotide) comprising a crNucleotide sequence linked to a tracrNucleotide sequence. The single guide polynucleotide comprises a first nucleotide sequence domain (referred to as Variable Targeting domain or VT domain) that can hybridize to a nucleotide sequence in a target DNA and a Cas9 endonuclease recognition domain (CER domain), that interacts with a Cas9 endonuclease polypeptide. By “domain” it is meant a contiguous stretch of nucleotides that can be RNA, DNA, and/or RNA-DNA-combination sequence. The VT domain and/or the CER domain of a single guide polynucleotide can comprise a RNA sequence, a DNA sequence, or a RNA-DNA-combination sequence. The single guide polynucleotide being comprised of sequences from the crNucleotide and the tracrNucleotide may be referred to as “single guide RNA” (when composed of a contiguous stretch of RNA nucleotides) or “single guide DNA” (when composed of a contiguous stretch of DNA nucleotides) or “single guide RNA-DNA” (when composed of a combination of RNA and DNA nucleotides). The single guide polynucleotide can form a complex with a Cas9 endonuclease, wherein said guide polynucleotide/Cas9 endonuclease complex (also referred to as a guide polynucleotide/Cas9 endonuclease system) can direct the Cas9 endonuclease to a genomic target site, enabling the Cas9 endonuclease to recognize, bind to, and optionally nick or cleave (introduce a single or double strand break) the target site. (See also U.S. Patent Application US 2015-0082478 A1, published on Mar. 19, 2015 and US 2015-0059010 A1, published on Feb. 26, 2015, both are hereby incorporated in its entirety by reference.)

A chimeric non-naturally occurring single guide RNA (sgRNA) includes a sgRNA that comprises regions that are not found together in nature (i.e., they are heterologous with each other). For example, a sgRNA comprising a first nucleotide sequence domain (referred to as Variable Targeting domain or VT domain) that can hybridize to a nucleotide sequence in a target DNA linked to a second nucleotide sequence (also referred to as a tracr mate sequence) that are not found linked together in nature.

The term “variable targeting domain” or “VT domain” is used interchangeably herein and includes a nucleotide sequence that can hybridize (is complementary) to one strand (nucleotide sequence) of a double strand DNA target site. The % complementation between the first nucleotide sequence domain (VT domain) and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The variable targeting domain can be at least 12, 13, 14, 15, 16, 17, 18, 19, 20, 21, 22, 23, 24, 25, 26, 27, 28, 29 or 30 nucleotides in length.

In some embodiments, the variable targeting domain comprises a contiguous stretch of 12 to 30, 12 to 29, 12 to 28, 12 to 27, 12 to 26, 12 to 25, 12 to 26, 12 to 25, 12 to 24, 12 to 23, 12 to 22, 12 to 21, 12 to 20, 12 to 19, 12 to 18, 12 to 17, 12 to 16, 12 to 15, 12 to 14, 12 to 13, 13 to 30, 13 to 29, 13 to 28, 13 to 27, 13 to 26, 13 to 25, 13 to 26, 13 to 25, 13 to 24, 13 to 23, 13 to 22, 13 to 21, 13 to 20, 13 to 19, 13 to 18, 13 to 17, 13 to 16, 13 to 15, 13 to 14, 14 to 30, 14 to 29, 14 to 28, 14 to 27, 14 to 26, 14 to 25, 14 to 26, 14 to 25, 14 to 24, 14 to 23, 14 to 22, 14 to 21, 14 to 20, 14 to 19, 14 to 18, 14 to 17, 14 to 16, 14 to 15, 15 to 30, 15 to 29, 15 to 28, 15 to 27, 15 to 26, 15 to 25, 15 to 26, 15 to 25, 15 to 24, 15 to 23, 15 to 22, 15 to 21, 15 to 20, 15 to 19, 15 to 18, 15 to 17, 15 to 16, 16 to 30, 16 to 29, 16 to 28, 16 to 27, 16 to 26, 16 to 25, 16 to 24, 16 to 23, 16 to 22, 16 to 21, 16 to 20, 16 to 19, 16 to 18, 16 to 17, 17 to 30, 17 to 29, 17 to 28, 17 to 27, 17 to 26, 17 to 25, 17 to 24, 17 to 23, 17 to 22, 17 to 21, 17 to 20, 17 to 19, 17 to 18, 18 to 30, 18 to 29, 18 to 28, 18 to 27, 18 to 26, 18 to 25, 18 to 24, 18 to 23, 18 to 22, 18 to 21, 18 to 20, 18 to 19, 19 to 30, 19 to 29, 19 to 28, 19 to 27, 19 to 26, 19 to 25, 19 to 24, 19 to 23, 19 to 22, 19 to 21, 19 to 20, 20 to 30, 20 to 29, 20 to 28, 20 to 27, 20 to 26, 20 to 25, 20 to 24, 20 to 23, 20 to 22, 20 to 21, 21 to 30, 21 to 29, 21 to 28, 21 to 27, 21 to 26, 21 to 25, 21 to 24, 21 to 23, 21 to 22, 22 to 30, 22 to 29, 22 to 28, 22 to 27, 22 to 26, 22 to 25, 22 to 24, 22 to 23, 23 to 30, 23 to 29, 23 to 28, 23 to 27, 23 to 26, 23 to 25, 23 to 24, 24 to 30, 24 to 29, 24 to 28, 24 to 27, 24 to 26, 24 to 25, 25 to 30, 25 to 29, 25 to 28, 25 to 27, 25 to 26, 26 to 30, 26 to 29, 26 to 28 nucleotides.

The variable targeting domain can be composed of a DNA sequence, a RNA sequence, a modified DNA sequence, a modified RNA sequence, or any combination thereof.

The term “Cas endonuclease recognition domain” or “CER domain” (of a guide polynucleotide) is used interchangeably herein and includes a nucleotide sequence that interacts with a Cas9 endonuclease polypeptide. A CER domain comprises a tracrNucleotide mate sequence followed by a tracrNucleotide sequence. The CER domain can be composed of a DNA sequence, a RNA sequence, a modified DNA sequence, a modified RNA sequence (see for example US 2015-0059010 A1, published on Feb. 26, 2015, incorporated in its entirety by reference herein), or any combination thereof.

The nucleotide sequence linking the crNucleotide and the tracrNucleotide of a single guide polynucleotide can comprise a RNA sequence, a DNA sequence, or a RNA-DNA combination sequence. In one embodiment, the nucleotide sequence linking the crNucleotide and the tracrNucleotide of a single guide polynucleotide (also referred to as loop) can be at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 78, 79, 80, 81, 82, 83, 84, 85, 86, 87, 88, 89, 90, 91, 92, 93, 94, 95, 96, 97, 98, 99 or 100 nucleotides in length. In some embodiments, the loop can be 3-4, 3-5, 3-6, 3-7, 3-8, 3-9, 3-10, 3-11, 3-12, 3-13, 3-14, 3-15, 3-20, 3-30, 3-40, 3-50, 3-60, 3-70, 3-80, 3-90, 3-100, 4-5, 4-6, 4-7, 4-8, 4-9, 4-10, 4-11, 4-12, 4-13, 4-14, 4-15, 4-20, 4-30, 4-40, 4-50, 4-60, 4-70, 4-80, 4-90, 4-100, 5-6, 5-7, 5-8, 5-9, 5-10, 5-11, 5-12, 5-13, 5-14, 5-15, 5-20, 5-30, 5-40, 5-50, 5-60, 5-70, 5-80, 5-90, 5-100, 6-7, 6-8, 6-9, 6-10, 6-11, 6-12, 6-13, 6-14, 6-15, 6-20, 6-30, 6-40, 6-50, 6-60, 6-70, 6-80, 6-90, 6-100, 7-8, 7-9, 7-10, 7-11, 7-12, 7-13, 7-14, 7-15, 7-20, 7-30, 7-40, 7-50, 7-60, 7-70, 7-80, 7-90, 7-100, 8-9, 8-10, 8-11, 8-12, 8-13, 8-14, 8-15, 8-20, 8-30, 8-40, 8-50, 8-60, 8-70, 8-80, 8-90, 8-100, 9-10, 9-11, 9-12, 9-13, 9-14, 9-15, 9-20, 9-30, 9-40, 9-50, 9-60, 9-70, 9-80, 9-90, 9-100, 10-20, 20-30, 30-40, 40-50, 50-60, 70-80, 80-90 or 90-100 nucleotides in length.

In one embodiment, the nucleotide sequence linking the crNucleotide and the tracrNucleotide of a single guide polynucleotide can comprise a tetraloop sequence, such as, but not limiting to a GAAA tetraloop sequence.

The guide polynucleotide can be produced by any method known in the art, including chemically synthesizing guide polynucleotides (such as but not limiting to Hendel et al. 2015, Nature Biotechnology 33, 985-989), in vitro generated guide polynucleotides, and/or self-splicing guide RNAs (such as but not limiting to Xie et al. 2015, PNAS 112:3570-3575).

A method of expressing RNA components such as gRNA in eukaryotic cells for performing Cas9-mediated DNA targeting has been to use RNA polymerase III (Pol III) promoters, which allow for transcription of RNA with precisely defined, unmodified, 5′- and 3′-ends (DiCarlo et al., Nucleic Acids Res. 41: 4336-4343; Ma et al., Mol. Ther. Nucleic Acids 3:e161). This strategy has been successfully applied in cells of several different species including maize and soybean (US 20150082478, published on Mar. 19, 2015). Methods for expressing RNA components that do not have a 5′ cap have been described (WO 2016/025131, published on Feb. 18, 2016).

In some embodiments, a subject nucleic acid (e.g., a guide polynucleotide, a nucleic acid comprising a nucleotide sequence encoding a guide polynucleotide; a nucleic acid encoding Cas9 endonuclease of the present disclosure; a crRNA or a nucleotide encoding a crRNA, a tracrRNA or a nucleotide encoding a tracrRNA, a nucleotide encoding a VT domain, a nucleotide encoding a CER domain, etc.) comprises a modification or sequence that provides for an additional desirable feature (e.g., modified or regulated stability; subcellular targeting; tracking, e.g., a fluorescent label; a binding site for a protein or protein complex; etc.). Nucleotide sequence modification of the guide polynucleotide, VT domain and/or CER domain can be selected from, but not limited to, the group consisting of a 5′ cap, a 3′ polyadenylated tail, a riboswitch sequence, a stability control sequence, a sequence that forms a dsRNA duplex, a modification or sequence that targets the guide poly nucleotide to a subcellular location, a modification or sequence that provides for tracking, a modification or sequence that provides a binding site for proteins, a Locked Nucleic Acid (LNA), a 5-methyl dC nucleotide, a 2,6-Diaminopurine nucleotide, a 2′-Fluoro A nucleotide, a 2′-Fluoro U nucleotide; a 2′-O-Methyl RNA nucleotide, a phosphorothioate bond, linkage to a cholesterol molecule, linkage to a polyethylene glycol molecule, linkage to a spacer 18 molecule, a 5′ to 3′ covalent linkage, or any combination thereof. These modifications can result in at least one additional beneficial feature, wherein the additional beneficial feature is selected from the group of a modified or regulated stability, a subcellular targeting, tracking, a fluorescent label, a binding site for a protein or protein complex, modified binding affinity to complementary target sequence, modified resistance to cellular degradation, and increased cellular permeability.

In one embodiment of the disclosure, the composition comprises at least one a single guide RNA capable of forming a guide RNA/Cas9 endonuclease complex, wherein said guide RNA/Cas9 endonuclease complex can recognize, bind to, and optionally nick or cleave a target sequence, wherein said single guide RNA is selected from the group consisting of SEQ ID NOs: 185-207, a functional fragment of SEQ ID NOs: 185-207, and a functional variant of SEQ ID NOs: 185-207.

In one embodiment of the disclosure, the composition is a single guide RNA capable of forming a guide RNA/Cas9 endonuclease complex, wherein said guide RNA/Cas9 endonuclease complex can recognize, bind to, and optionally nick or cleave a target sequence, wherein said single guide RNA comprises a chimeric non-naturally occurring crRNA linked to a tracrRNA, wherein said tracrRNA comprises a nucleotide sequence selected from the group consisting of SEQ ID NOs: 139-184, a functional fragment of SEQ ID NOs: 139-184, and a functional variant of SEQ ID NOs: 139-184.

The guide RNA can also be a dual molecule comprising a chimeric non-naturally occurring crRNA linked to a tracrRNA, wherein said chimeric non-naturally occurring crRNA comprises a nucleotide sequence selected from the group consisting of SEQ ID NOs: 116-138, a functional fragment of SEQ ID NOs: 116-138, and a functional variant of SEQ ID NOs: 116-138, and/or wherein said tracrRNA comprises a nucleotide sequence selected from the group consisting of SEQ ID NOs: 139-184, a functional fragment of SEQ ID NOs: 139-184, and a functional variant of SEQ ID NOs: 139-184.

In one embodiment of the disclosure, the composition is a single guide RNA capable of forming a guide RNA/Cas9 endonuclease complex, wherein said guide RNA/Cas9 endonuclease complex can recognize, bind to, and optionally nick or cleave a target sequence, wherein said single guide RNA comprises a chimeric non-naturally occurring crRNA linked to a tracrRNA, wherein said chimeric non-naturally occurring crRNA comprises a nucleotide sequence selected from the group consisting of SEQ ID NOs: 116-138, a functional fragment of SEQ ID NOs: 116-138, and a functional variant of SEQ ID NOs: 116-138.

Single guide RNAs targeting a target site in the genome of an organism can be designed by changing Variable Targeting Domain (VT) of any one of SEQ ID NOs: 185-207 (or a functional fragment of SEQ ID NOs: 185-207, or a functional variant of SEQ ID NOs: 185-207) with any random nucleotide that can hybridize to any desired target sequence. In SEQ ID NOs: 185-207 the sgRNA comprises a VT domain of 20 Ns. As described herein, the variable targeting domain can be at least 12, 13, 14, 15, 16, 17, 18, 19, 20, 21, 22, 23, 24, 25, 26, 27, 28, 29 or 30 nucleotides in length. The VT domain of these sgRNAs can include

The terms “functional fragment ”, “fragment that is functionally equivalent” and “functionally equivalent fragment” of a single guide RNA, crRNA or tracrRNA are used interchangeably herein, and refer to a portion or subsequence of the single guide RNA, crRNA or tracrRNA, respectively, of the present disclosure in which the ability to function as a guide RNA, crRNA or tracrRNA, respectively, is retained.

A functional fragments of a guide RNA (guide polynucleotide) of the present disclosure include a fragment of 20-40, 20-45, 20-50, 20-55, 20-60, 20-65, 20-70, 20-75, 20-80, 25-40, 25-45, 25-50, 25-55, 25-60, 25-65, 25-70, 25-75, 25-80, 30-40, 30-45, 30-50, 30-55, 30-60, 30-65, 30-70, 30-75, 30-80, 35-40, 35-45, 35-50, 35-55, 35-60, 35-65, 35-70, 35-75, 35-80, 40-45, 40-50, 40-55, 40-60, 40-65, 40-70, 40-75, 40-80, 45-50, 45-55, 45-60, 45-65, 45-70, 45-75, 45-80, 50-55, 50-60, 50-65, 50-70, 50-75, 50-80, 55-55, 55-60, 55-65, 55-70, 55-75, 55-80, 60-65, 60-70, 60-75, 60-80, 65-70, 65-75, 65-80, 70-75, 70-80 or 75-80 nucleotides of a reference guide RNA, such as the reference guide RNAs of SEQ ID NOs: 185-207.

Functional fragments of a crRNA of the present disclosure include a fragment of 5-30, 10-30, 15-30, 20-30, 25-30, 5-25, 10-25, 15-25, 20-25, 5-20, 10-20, 15-20, 5-15, 10-15, nucleotides of a reference crRNA, such as the reference crRNAs of SEQ ID NOs: 116-138.

The terms “functional variant”, “Variant that is functionally equivalent” and “functionally equivalent variant” of a guide RNA, crRNA or tracrRNA (respectively) are used interchangeably herein, and refer to a variant of the guide RNA, crRNA or tracrRNA, respectively, of the present disclosure in which the ability to function as a guide RNA, crRNA or tracrRNA, respectively, is retained. A functional variant of a single guide RNA may comprise a nucleotide sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to reference single guide RNA, such as the reference single guide RNA of SEQ ID NOs:185-207, described herein. In some embodiments, a functional variant of a single guide RNA comprises a nucleotide sequence having at least about 60%, at least about 65%, at least about 70%, at least about 75%, at least about 80%, at least about 85%, at least about 90%, at least about 95%, at least about 98%, at least about 99%, or 100% nucleotide sequence identity over a stretch of at least 8, 9, 10, 11, 12, 13, 14, 15, 16, 17, 18, 19, 20, 21, 22, 23, 24, 25, 26, 27, 28, 29, 30 contiguous nucleotides to any one of the nucleotide sequences set forth in SEQ ID NOs: 185-207.

Functional variants of a guide polynucleotide of the present disclosure can comprise a modified guide polynucleotide wherein the modification comprises an engineered secondary structure, and/or an artificial loop, and/or a reduction in the length and/or degree of complementation in a region of hybridization compared to a region of hybridization of a reference guide polynucleotide, including the guide polynucleotides of SEQ ID NOs: 185-207, and/or a reduction in the length and/or degree of complementation in the portion of the protein-binding segment that forms a double stranded RNA duplex.

Functional variants of a guide polynucleotide of the present disclosure can comprise a modified guide polynucleotide wherein the modification comprises adding, removing, or otherwise altering loops and/or hairpins in the single guide RNA.

Functional variants of a guide polynucleotide of the present disclosure can comprise a modified guide polynucleotide wherein the modification comprises one or more modified nucleotides in the nucleotide sequence, wherein the one or more modified nucleotides comprises at least one non-naturally-occurring nucleotide, nucleotide mimetic (as described in US application US2014/0068797, published Mar. 6, 2014), or analog thereof, or wherein the one or more modified nucleotides are selected from the group consisting of 2′-0-methylanalogs, 2′-fluoro analogs 2-aminopurine, 5-bromo-uridine, pseudouridine, and 7-methylguanosine.

In one aspect, the functional variant of the guide RNA can form a guide RNA/Cas9 endonuclease complex that can recognize, bind to, and optionally nick or cleave a target sequence.

A functional variant of a crRNA may comprise an nucleotide sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to a reference crRNA, such as the reference crRNA of SEQ ID NOs:116-138, described herein. In one aspect, the functional variant of the crRNA can bind to a Cas9 endonuclease described herein and together with a tracrRNA, or as part of a guide RNA, can form a guide RNA/Cas9 endonuclease complex that can recognize, bind to, and optionally nick or cleave a target sequence.

A functional variant of a tracrRNA may comprise an nucleotide sequence that is at least about 80%, 81%, 82%, 83%, 84%, 85%, 86%, 87%, 88%, 89%, 90%, 91%, 92%, 93%, 94%, 95%, 96%, 97%, 98%, or 99% identical to a reference tracrRNA, such as the reference tracrRNA comprising a nucleotide sequence selected from the group consisting of SEQ ID NOs:139-184, described herein. In one aspect, the functional variant of the tracrRNA can bind to a Cas9 endonuclease described herein and together with a crRNA, or as part of a guide RNA, can form a guide RNA/Cas9 endonuclease complex that can recognize, bind to, and optionally nick or cleave a target sequence.

Methods for determining if fragments or variants of a guide RNA, crRNA or tracrRNA are functional include methods that measure the Cas9 endonuclease activity when in complex with said fragment/variant guide RNA, crRNA and/or tracrRNA, as described herein. Methods for measuring Cas9 endonuclease activity (either double strand breaks or single strand breaks) include methods that measure the mutation frequency at a target site, as described herein. If mutations are observed at the intended target sites when using a fragment or variant of guide RNA, crRNA and/or tracrRNA of the present disclosure, in complex with a cas9 endonuclease of the present disclosure, the fragments or variants are functional.

The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA). The single guide RNA can comprise a crRNA or crRNA fragment and a tracrRNA or tracrRNA fragment of the type II CRISPR/Cas9 system that can form a complex with a type II Cas9 endonuclease, wherein said guide RNA/Cas9 endonuclease complex can direct the Cas9 endonuclease to a DNA target site, enabling the Cas9 endonuclease to recognize, bind to, and optionally nick or cleave (introduce a single or double strand break) the DNA target site.

Single guide RNAs targeting a target site in the genome of an organism can be designed by changing the Variable Targeting Domain (VT) of anyone of SEQ ID NOs: 185-207 (or a functional fragment or functional variant of SEQ ID NOs: 185-207) with any random nucleotide that can hybridize to any desired target sequence.

The terms “guide RNA/Cas9 endonuclease complex”, “guide RNA/Cas9 endonuclease system”, “guide RNA/Cas9 complex”, “guide RNA/Cas9 system”, “gRNA/Cas9 complex”, “gRNA/Cas9 system”, “RNA-guided endonuclease”, “RGEN” are used interchangeably herein and refer to at least one RNA component and at least one Cas9 endonuclease that are capable of forming a complex, wherein said guide RNA/Cas9 endonuclease complex can direct the Cas9 endonuclease to a DNA target site, enabling the Cas9 endonuclease to recognize, bind to, and optionally nick or cleave (introduce a single or double strand break) the DNA target site. A guide RNA/Cas9 endonuclease complex herein can comprise Cas9 protein(s) and suitable RNA component(s) of any of the four known CRISPR systems (Horvath and Barrangou, 2010, Science 327:167-170) such as a type I, II, or III CRISPR system. A guide RNA/Cas9 endonuclease complex can comprise a Type II Cas9 endonuclease and at least one RNA component (e.g., a crRNA and tracrRNA, or a gRNA). (See also U.S. Patent Application US 2015-0082478 A1, published on Mar. 19, 2015 and US 2015-0059010 A1, published on Feb. 26, 2015, both are hereby incorporated in its entirety by reference).

The guide polynucleotide can be introduced into a cell transiently, as single stranded polynucleotide or a double stranded polynucleotide, using any method known in the art such as, but not limited to, particle bombardment, Agrobacterium transformation or topical applications. The guide polynucleotide can also be introduced indirectly into a cell by introducing a recombinant DNA molecule (via methods such as, but not limited to, particle bombardment or Agrobacterium transformation) comprising a heterologous nucleic acid fragment encoding a guide polynucleotide, operably linked to a specific promoter that is capable of transcribing the guide RNA in said cell. The specific promoter can be, but is not limited to, a RNA polymerase III promoter, which allow for transcription of RNA with precisely defined, unmodified, 5′- and 3′-ends (DiCarlo et al., Nucleic Acids Res. 41: 4336-4343; Ma et al., Mol. Ther. Nucleic Acids 3:e161) as described in U.S. application 62/036,652, filed on Aug. 13, 2014, incorporated herein in its entirety by reference.

Direct delivery of a polynucleotide modification template into plant cells can be achieved through particle mediated delivery, and any other direct method of delivery, such as but not limiting to, polyethylene glycol (PEG)-mediated transfection to protoplasts, whiskers mediated transformation, electroporation, particle bombardment, cell-penetrating peptides, or mesoporous silica nanoparticle (MSN)-mediated direct protein delivery can be successfully used for delivering a polynucleotide modification template in eukaryotic cells, such as plant cells.

The donor DNA can be introduced by any means known in the art. The donor DNA may be provided by any transformation method known in the art including, for example, Agrobacterium-mediated transformation or biolistic particle bombardment. The donor DNA may be present transiently in the cell or it could be introduced via a viral replicon. In the presence of the Cas9 endonuclease and the target site, the donor DNA is inserted into the transformed plant's genome.

Direct delivery of any one of the guided Cas9 system components can be accompanied by direct delivery (co-delivery) of other mRNAs that can promote the enrichment and/or visualization of cells receiving the guide polynucleotide/Cas9 endonuclease complex components. For example, direct co-delivery of the guide polynucleotide/Cas9 endonuclease components (and/or guide polynucleotide/Cas9 endonuclease complex itself) together with mRNA encoding phenotypic markers (such as but not limiting to transcriptional activators such as CRC (Bruce et al. 2000 The Plant Cell 12:65-79) can enable the selection and enrichment of cells without the use of an exogenous selectable marker by restoring function to a non-functional gene product as described in PCT/US16/57272 filed Oct. 17, 2016 and PCT/US16/57279, filed Oct. 17, 2016, both incorporated herein by reference.

In one aspect, the guide polynucleotide/Cas9 endonuclease complex of the present disclosure comprises a guide RNA of the present disclosure (such as a single guide RNA selected from the group consisting of SEQ ID NOs: 185-207, a functional fragment of SEQ ID NOs: 185-207, and a functional variant of SEQ ID NOs: 185-207) in complex with a Cas9 endonuclease of the present disclosure (such as a Cas9 endonuclease selected from the group consisting of SEQ ID NOs: 47-69, a functional fragment of SEQ ID NOs: 47-69, and a functional variant of SEQ ID NOs: 47-69).

The terms “target site”, “target sequence”, “target site sequence, “target DNA”, “target locus”, “genomic target site”, “genomic target sequence”, “genomic target locus” and “protospacer”, are used interchangeably herein and refer to a polynucleotide sequence such as, but not limited to, a nucleotide sequence on a chromosome, episome, or any other DNA molecule in the genome (including chromosomal, choloroplastic, mitochondrial DNA, plasmid DNA) of a cell, at which a guide polynucleotide/Cas9 endonuclease complex can recognize, bind to, and optionally nick or cleave. The target site can be an endogenous site in the genome of a cell, or alternatively, the target site can be heterologous to the cell and thereby not be naturally occurring in the genome of the cell, or the target site can be found in a heterologous genomic location compared to where it occurs in nature. As used herein, terms “endogenous target sequence” and “native target sequence” are used interchangeable herein to refer to a target sequence that is endogenous or native to the genome of a cell and is at the endogenous or native position of that target sequence in the genome of the cell. Cells include, but are not limited to, human, non-human, animal, bacterial, fungal, insect, yeast, non-conventional yeast, and plant cells as well as plants and seeds produced by the methods described herein. An “artificial target site” or “artificial target sequence” are used interchangeably herein and refer to a target sequence that has been introduced into the genome of a cell. Such an artificial target sequence can be identical in sequence to an endogenous or native target sequence in the genome of a cell but be located in a different position (i.e., a non-endogenous or non-native position) in the genome of a cell.

An “altered target site”, “altered target sequence”, “modified target site”, “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, or (iv) any combination of (i)-(iii).

Methods for “modifying a target site” and “altering a target site” are used interchangeably herein and refer to methods for producing an altered target site.

The length of the target DNA sequence (target site) can vary, and includes, for example, target sites that are at least 12, 13, 14, 15, 16, 17, 18, 19, 20, 21, 22, 23, 24, 25, 26, 27, 28, 29, 30 or more nucleotides in length. It is further possible that the target site can be palindromic, that is, the sequence on one strand reads the same in the opposite direction on the complementary strand. The nick/cleavage site can be within the target sequence or the nick/cleavage site could be outside of the target sequence. In another variation, the cleavage could occur at nucleotide positions immediately opposite each other to produce a blunt end cut or, in other cases, the incisions could be staggered to produce single-stranded overhangs, also called “sticky ends”, which can be either 5′ overhangs, or 3′ overhangs. Active variants of genomic target sites can also be used. Such active variants can comprise at least 65%, 70%, 75%, 80%, 85%, 90%, 91%, 92%, 93%, 94%, 95%, 96%, 97%, 98%, 99% or more sequence identity to the given target site, wherein the active variants retain biological activity and hence are capable of being recognized and cleaved by an Cas9 endonuclease. Assays to measure the single or double-strand break of a target site by an endonuclease are known in the art and generally measure the overall activity and specificity of the agent on DNA substrates containing recognition sites.

A “protospacer adjacent motif” (PAM) herein refers to a short nucleotide sequence adjacent to a target sequence (protospacer) that is recognized (targeted) by a guide polynucleotide/Cas9 endonuclease system described herein. The Cas9 endonuclease may not successfully recognize a target DNA sequence if the target DNA sequence is not followed by a PAM sequence. The sequence and length of a PAM herein can differ depending on the Cas9 protein or Cas9 protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

A “randomized PAM” and “randomized protospacer adjacent motif” are used interchangeably herein, and refer to a random DNA sequence adjacent to a target sequence (protospacer) that is recognized (targeted) by a guide polynucleotide/Cas9 endonuclease system described herein. The randomized PAM sequence can be of any length but is typically 1, 2, 3, 4, 5, 6, 7, 8, 9, 10, 11, 12, 13, 14, 15, 16, 17, 18, 19 or 20 nucleotides long. A randomized nucleotide includes anyone of the nucleotides A, C, G or T.

Given the diversity of Type II CRISPR-Cas systems (Fonfara et al. (2014) Nucleic Acids Res. 42:2577-2590), it is plausible that many of the Cas9 endonucleases and cognate guide RNAs may have unique sequence recognition and enzymatic properties different from those previously described or characterized. For example, cleavage activity and specificity may be enhanced or proto-spacer adjacent motif (PAM) sequence may be different leading to increased genomic target site density. To tap into this vast unexplored diversity and expand the repertoire of Cas9 endonucleases and cognate guide RNAs available for genome targeting, the components of Cas9 target site recognition, the PAM sequence and the guide RNA (either duplexed CRISPR RNA (crRNA) and trans-activating CRISPR RNA (tracrRNA) or chimeric fusion of crRNA and tracrRNA (single guide RNA (sgRNA), need to be established for each new system. As described herein, CRISPR-Cas loci (including Cas9 genes and open reading frames, CRISPR array and anti-repeats) from uncharacterized CRISPR-Cas systems were identified by searching internal Pioneer-DuPont databases consisting of microbial genomes. The Cas9 endonuclease described herein can be expressed and purified by methods known in the art. As described herein, the transcriptional direction of the tracrRNA for all the CRISPR-Cas systems can be deduced (as described in PCT/US16/32028 filed May 12, 2016, and PCT/US16/32073 filed May 12, 2016), and examples of sgRNAs (described herein, see SEQ ID NOs:185-207) and its components (VT, crRNA repeat, loop, anti-repeat and 3′tracrRNA) were identified for each new CRISPR-Cas endonuclease described herein.

The terms “targeting”, “gene targeting” and “DNA targeting” are used interchangeably herein. DNA targeting herein may be the specific introduction of a knock-out, edit, or knock-in at a particular DNA sequence, such as in a chromosome or plasmid of a cell. In general, DNA targeting can be performed herein by cleaving one or both strands at a specific DNA sequence in a cell with an endonuclease associated with a suitable polynucleotide component. Such DNA cleavage, if a double-strand break (DSB) can prompt NHEJ or HDR processes which can lead to modifications at the target site.

A targeting method herein can be performed in such a way that two or more DNA target sites are targeted in the method, for example. Such a method can optionally be characterized as a multiplex method. Two, three, four, five, six, seven, eight, nine, ten, or more target sites can be targeted at the same time in certain embodiments. A multiplex method is typically performed by a targeting method herein in which multiple different RNA components are provided, each designed to guide a guide polynucleotide/Cas9 endonuclease complex to a unique DNA target site.

The terms “knock-out”, “gene knock-out” and “genetic knock-out” are used interchangeably herein. A knock-out represents a DNA sequence of a cell that has been rendered partially or completely inoperative by targeting with a Cas9 protein; such a DNA sequence prior to knock-out could have encoded an amino acid sequence, or could have had a regulatory function (e.g., promoter), for example. A knock-out may be produced by an indel (insertion or deletion of nucleotide bases in a target DNA sequence through NHEJ), or by specific removal of sequence that reduces or completely destroys the function of sequence at or near the targeting site.

In one embodiment of the disclosure, the method comprises a method for modifying a target site in the genome of a cell, the method comprising introducing into said cell at least one guide RNA and at least one Cas9 endonuclease selected from the group consisting of SEQ ID NOs: 47-69, a functional fragment of SEQ ID NOs: 47-69, and a functional variant of SEQ ID NOs: 47-69, wherein said guide RNA and Cas9 endonuclease can form a complex that is capable of recognizing, binding to, and optionally nicking or cleaving all or part of said target site. The method can further comprise identifying at least one cell that has a modification at said target, wherein the modification at said target site is selected from the group consisting of (i) a replacement of at least one nucleotide, (ii) a deletion of at least one nucleotide, (iii) an insertion of at least one nucleotide, and (iv) any combination of (i)-(iii).

The guide polynucleotide/Cas9 endonuclease system can be used in combination with a co-delivered polynucleotide modification template to allow for editing (modification) of a genomic nucleotide sequence of interest. (See also U.S. Patent Application US 2015-0082478 A1, published on Mar. 19, 2015 and WO2015/026886 A1, published on Feb. 26, 2015, both are hereby incorporated in its entirety by reference.)

A “modified nucleotide” or “edited nucleotide” refers to a nucleotide sequence of interest that comprises at least one alteration when compared to its non-modified nucleotide sequence. Such “alterations” include, for example: (i) replacement of at least one nucleotide, (ii) a deletion of at least one nucleotide, (iii) an insertion of at least one nucleotide, or (iv) any combination of (i)-(iii).

The term “polynucleotide modification template” includes a polynucleotide that comprises at least one nucleotide modification when compared to the nucleotide sequence to be edited. A nucleotide modification can be at least one nucleotide substitution, addition or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

In one embodiment, the disclosure describes a method for editing a nucleotide sequence in the genome of a cell, the method comprising introducing into said cell a polynucleotide modification template, at least one guide RNA and at least one Cas9 endonuclease selected from the group consisting of SEQ ID NOs: 47-69, a functional fragment of SEQ ID NOs: 47-69, and a functional variant of SEQ ID NOs: 47-69, wherein said polynucleotide modification template comprises at least one nucleotide modification of said nucleotide sequence, wherein said guide RNA and Cas9 endonuclease can form a complex that is capable of recognizing, binding to, and optionally nicking or cleaving all or part of said target site.

Cells include, but are not limited to, human, non-human, animal, bacterial, fungal, insect, yeast, non-conventional yeast, and plant cells as well as plants and seeds produced by the methods described herein. Plant cells include cells selected from the group consisting of maize, rice, sorghum, rye, barley, wheat, millet, oats, sugarcane, turfgrass, or switchgrass, soybean, canola, alfalfa, sunflower, cotton, tobacco, peanut, potato, tobacco, Arabidopsis, and safflower cells. The nucleotide to be edited can be located within or outside a target site recognized and cleaved by a Cas9 endonuclease.

In one embodiment, the at least one nucleotide modification is not a modification at a target site recognized and cleaved by a Cas9 endonuclease. In another embodiment, the nucleotide modification is located in close proximity to the target site. In another embodiment, there are at least 1, 2, 3, 4, 5, 6, 7, 8, 9, 10, 11, 12, 13, 14, 15, 16, 17, 18, 19, 20, 21, 22, 23, 24, 25, 26, 27, 30, 40, 50, 100, 200, 300, 400, 500, 600, 700, 900 or 1000 nucleotides between the at least one nucleotide to be edited and the genomic target site.

Genome editing can be accomplished using any method of gene editing available. For example, gene editing can be accomplished through the introduction into a host cell of a polynucleotide modification template (sometimes also referred to as a gene repair oligonucleotide) containing a targeted modification to a gene within the genome of the host cell. The polynucleotide modification template for use in such methods can be either single-stranded or double-stranded. Examples of such methods are generally described, for example, in US Publication No. 2013/0019349.

In some embodiments, gene editing may be facilitated through the induction of a double-stranded break (DSB) in a defined position in the genome near the desired alteration. DSBs can be induced using any DSB-inducing agent available, including, but not limited to, TALENs, meganucleases, zinc finger nucleases, Cas9-gRNA systems (based on bacterial CRISPR-Cas systems), and the like. In some embodiments, the introduction of a DSB can be combined with the introduction of a polynucleotide modification template.

The process for editing a genomic sequence combining DSB and modification templates generally comprises: introducing into a host cell, a DSB-inducing agent, or a nucleic acid encoding a DSB-inducing agent, that recognizes a target sequence in the chromosomal sequence and is able to induce a DSB in the genomic sequence, and at least one polynucleotide modification template comprising at least one nucleotide alteration when compared to the nucleotide sequence to be edited. The polynucleotide modification template can further comprise nucleotide sequences flanking the at least one nucleotide alteration, in which the flanking sequences are substantially homologous to the chromosomal region flanking the DSB. Genome editing using DSB-inducing agents, such as Cas9-gRNA complexes, has been described, for example in U.S. Patent Application US 2015-0082478 A1, published on Mar. 19, 2015, WO2015/026886 A1, published on Feb. 26, 2015, U.S. application 62/023,246, filed on Jul. 7, 2014, and U.S. application 62/036,652, filed on Aug. 13, 2014, all of which are incorporated by reference herein.

The terms “knock-in”, “gene knock-in, “gene insertion” and “genetic knock-in” are used interchangeably herein. A knock-in represents the replacement or insertion of a DNA sequence at a specific DNA sequence in cell by targeting with a Cas9 protein (by HR, wherein a suitable donor DNA polynucleotide is also used). Examples of knock-ins are a specific insertion of a heterologous amino acid coding sequence in a coding region of a gene, or a specific insertion of a transcriptional regulatory element in a genetic locus.

Various methods and compositions can be employed to obtain a cell or organism having a polynucleotide of interest inserted in a target site for a Cas9 endonuclease. Such methods can employ homologous recombination to provide integration of the polynucleotide of Interest at the target site. In one method provided, a polynucleotide of interest is introduced into the organism cell in a donor DNA construct. As used herein, “donor DNA” is a DNA construct that comprises a polynucleotide of Interest to be inserted into the target site of a Cas9 endonuclease. The donor DNA construct further comprises a first and a second region of homology that flank the polynucleotide of Interest. The first and second regions of homology of the donor DNA share homology to a first and a second genomic region, respectively, present in or flanking the target site of the cell or organism genome. By “homology” is meant DNA sequences that are similar. For example, a “region of homology to a genomic region” that is found on the donor DNA is a region of DNA that has a similar sequence to a given “genomic region” in the cell or organism genome. A region of homology can be of any length that is sufficient to promote homologous recombination at the cleaved target site. For example, the region of homology can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800, 5-2900, 5-3000, 5-3100 or more bases in length such that the region of homology has sufficient homology to undergo homologous recombination with the corresponding genomic region. “Sufficient homology” indicates that two polynucleotide sequences have sufficient structural similarity to act as substrates for a homologous recombination reaction. The structural similarity includes overall length of each polynucleotide fragment, as well as the sequence similarity of the polynucleotides. Sequence similarity can be described by the percent sequence identity over the whole length of the sequences, and/or by conserved regions comprising localized similarities such as contiguous nucleotides having 100% sequence identity, and percent sequence identity over a portion of the length of the sequences.

The amount of homology or sequence identity shared by a target and a donor polynucleotide can vary and includes total lengths and/or regions having unit integral values in the ranges of about 1-20 bp, 20-50 bp, 50-100 bp, 75-150 bp, 100-250 bp, 150-300 bp, 200-400 bp, 250-500 bp, 300-600 bp, 350-750 bp, 400-800 bp, 450-900 bp, 500-1000 bp, 600-1250 bp, 700-1500 bp, 800-1750 bp, 900-2000 bp, 1-2.5 kb, 1.5-3 kb, 2-4 kb, 2.5-5 kb, 3-6 kb, 3.5-7 kb, 4-8 kb, 5-10 kb, or up to and including the total length of the target site. These ranges include every integer within the range, for example, the range of 1-20 bp includes 1, 2, 3, 4, 5, 6, 7, 8, 9, 10, 11, 12, 13, 14, 15, 16, 17, 18, 19 and 20 bps. The amount of homology can also described by percent sequence identity over the full aligned length of the two polynucleotides which includes percent sequence identity of about at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Sufficient homology includes any combination of polynucleotide length, global percent sequence identity, and optionally conserved regions of contiguous nucleotides or local percent sequence identity, for example sufficient homology can be described as a region of 75-150 bp having at least 80% sequence identity to a region of the target locus. Sufficient homology can also be described by the predicted ability of two polynucleotides to specifically hybridize under high stringency conditions, see, for example, Sambrook et al., (1989) Molecular Cloning: A Laboratory Manual, (Cold Spring Harbor Laboratory Press, NY); Current Protocols in Molecular Biology, Ausubel et al., Eds (1994) Current Protocols, (Greene Publishing Associates, Inc. and John Wiley & Sons, Inc.); and, Tijssen (1993) Laboratory Techniques in Biochemistry and Molecular Biology—Hybridization with Nucleic Acid Probes, (Elsevier, New York).

In one embodiment of the disclosure, the method comprises a method for modifying a target site in the genome of a cell, the method comprising introducing into said cell at least one guide RNA, at least one donor DNA, and at least one Cas9 endonuclease selected from the group consisting of SEQ ID NOs: 47-69, a functional fragment of SEQ ID NOs: 47-69, and a functional variant of SEQ ID NOs: 47-69, wherein said at least one guide RNA and at least one Cas9 endonuclease can form a complex that is capable of recognizing, binding to, and optionally nicking or cleaving all or part of said target site, wherein said donor DNA comprises a polynucleotide of interest.

The guide polynucleotide/Cas9 endonuclease systems described herein can be used for introducing one or more polynucleotides of interest or one or more traits of interest into one or more target sites by introducing one or more guide polynucleotides, one Cas endonuclease, and optionally one or more donor DNAs into a plant cell (as described in U.S. patent application Ser. No. 14/463,687, file Aug. 20, 2014, incorporated by reference herein). A fertile plant can be produced from that plant cell that comprises an alteration at said one or more target sites, wherein the alteration is selected from the group consisting of (i) replacement of at least one nucleotide, (ii) a deletion of at least one nucleotide, (iii) an insertion of at least one nucleotide, and (iv) any combination of (i)-(iii). Plants comprising these altered target sites can be crossed with plants comprising at least one gene or trait of interest in the same complex trait locus; thereby further stacking traits in said complex trait locus (see also US-2013-0263324-A1, published 3 Oct. 2013 and in PCT/US13/22891, published Jan. 24, 2013).

As used herein, a “genomic region” is a segment of a chromosome in the genome of a cell that is present on either side of the target site or, alternatively, also comprises a portion of the target site. The genomic region can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800. 5-2900, 5-3000, 5-3100 or more bases such that the genomic region has sufficient homology to undergo homologous recombination with the corresponding region of homology.

Polynucleotides of interest and/or traits can be stacked together in a complex trait locus as described in US 2013/0263324-A1, published Oct. 3, 2013 and in PCT/US13/22891, published Jan. 24, 2013, both applications are hereby incorporated by reference. The guide polynucleotide/Cas9 endonuclease system described herein provides for an efficient system to generate double strand breaks and allows for traits to be stacked in a complex trait locus.

The structural similarity between a given genomic region and the corresponding region of homology found on the donor DNA can be any degree of sequence identity that allows for homologous recombination to occur. For example, the amount of homology or sequence identity shared by the “region of homology” of the donor DNA and the “genomic region” of the organism genome can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination

The region of homology on the donor DNA can have homology to any sequence flanking the target site. While in some embodiments the regions of homology share significant sequence homology to the genomic sequence immediately flanking the target site, it is recognized that the regions of homology can be designed to have sufficient homology to regions that may be further 5′ or 3′ to the target site. In still other embodiments, the regions of homology can also have homology with a fragment of the target site along with downstream genomic regions. In one embodiment, the first region of homology further comprises a first fragment of the target site and the second region of homology comprises a second fragment of the target site, wherein the first and second fragments are dissimilar.

As used herein, “homologous recombination” includes the exchange of DNA fragments between two DNA molecules at the sites of homology. The frequency of homologous recombination is influenced by a number of factors. Different organisms vary with respect to the amount of homologous recombination and the relative proportion of homologous to non-homologous recombination. Generally, the length of the region of homology affects the frequency of homologous recombination events: the longer the region of homology, the greater the frequency. The length of the homology region needed to observe homologous recombination is also species-variable. In many cases, at least 5 kb of homology has been utilized, but homologous recombination has been observed with as little as 25-50 bp of homology. See, for example, Singer et al., (1982) Cell 31:25-33; Shen and Huang, (1986) Genetics 112:441-57; Watt et al., (1985) Proc. Natl. Acad. Sci. USA 82:4768-72, Sugawara and Haber, (1992) Mol Cell Biol 12:563-75, Rubnitz and Subramani, (1984) Mol Cell Biol 4:2253-8; Ayares et al., (1986) Proc. Natl. Acad. Sci. USA 83:5199-203; Liskay et al., (1987) Genetics 115:161-7.

Homology-directed repair (HDR) is a mechanism in cells to repair double-stranded and single stranded DNA breaks. Homology-directed repair includes homologous recombination (HR) and single-strand annealing (SSA) (Lieber. 2010 Annu. Rev. Biochem. 79:181-211). The most common form of HDR is called homologous recombination (HR), which has the longest sequence homology requirements, between the donor and acceptor DNA. Other forms of HDR include single-stranded annealing (SSA) and breakage-induced replication, and these require shorter sequence homology relative to HR. Homology-directed repair at nicks (single-stranded breaks) can occur via a mechanism distinct from HDR at double-strand breaks (Davis and MaizeIs (2014) PNAS (0027-8424), 111 (10), p. E924-E932).

Alteration of the genome of a plant cell, for example, through homologous recombination (HR), is a powerful tool for genetic engineering. Homologous recombination has been demonstrated in plants (Halfter et al., (1992) Mol Gen Genet 231:186-93) and insects (Dray and Gloor, 1997, Genetics 147:689-99). Homologous recombination has also been accomplished in other organisms. For example, at least 150-200 bp of homology was required for homologous recombination in the parasitic protozoan Leishmania (Papadopoulou and Dumas, (1997) Nucleic Acids Res 25:4278-86). In the filamentous fungus Aspergillus nidulans, gene replacement has been accomplished with as little as 50 bp flanking homology (Chaveroche et al., (2000) Nucleic Acids Res 28:e97). Targeted gene replacement has also been demonstrated in the ciliate Tetrahymena thermophila (Gaertig et al., (1994) Nucleic Acids Res 22:5391-8). In mammals, homologous recombination has been most successful in the mouse using pluripotent embryonic stem cell lines (ES) that can be grown in culture, transformed, selected and introduced into a mouse embryo (Watson et al., 1992, Recombinant DNA, 2nd Ed., (Scientific American Books distributed by WH Freeman & Co.).

Error-prone DNA repair mechanisms can produce mutations at double-strand break sites. The Non-Homologous-End-Joining (NHEJ) pathways are the most common repair mechanism to bring the broken ends together (Bleuyard et al., (2006) DNA Repair 5:1-12). The structural integrity of chromosomes is typically preserved by the repair, but deletions, insertions, or other rearrangements are possible. The two ends of one double-strand break are the most prevalent substrates of NHEJ (Kirik et al., (2000) EMBO J 19:5562-6), however if two different double-strand breaks occur, the free ends from different breaks can be ligated and result in chromosomal deletions (Siebert and Puchta, (2002) Plant Cell 14:1121-31), or chromosomal translocations between different chromosomes (Pacher et al., (2007) Genetics 175:21-9).

Episomal DNA molecules can also be ligated into the double-strand break, for example, integration of T-DNAs into chromosomal double-strand breaks (Chilton and Que, (2003) Plant Physiol 133:956-65; Salomon and Puchta, (1998) EMBO J 17:6086-95). Once the sequence around the double-strand breaks is altered, for example, by exonuclease activities involved in the maturation of double-strand breaks, gene conversion pathways can restore the original structure if a homologous sequence is available, such as a homologous chromosome in non-dividing somatic cells, or a sister chromatid after DNA replication (Molinier et al., (2004) Plant Cell 16:342-52). Ectopic and/or epigenic DNA sequences may also serve as a DNA repair template for homologous recombination (Puchta, (1999) Genetics 152:1173-81).

Once a double-strand break is induced in the DNA, the cell's DNA repair mechanism is activated to repair the break. Error-prone DNA repair mechanisms can produce mutations at double-strand break sites. The most common repair mechanism to bring the broken ends together is the nonhomologous end-joining (NHEJ) pathway (Bleuyard et al., (2006) DNA Repair 5:1-12). The structural integrity of chromosomes is typically preserved by the repair, but deletions, insertions, or other rearrangements are possible (Siebert and Puchta, (2002) Plant Cell 14:1121-31; Pacher et al., (2007) Genetics 175:21-9).

Alternatively, the double-strand break can be repaired by homologous recombination between homologous DNA sequences. Once the sequence around the double-strand break is altered, for example, by exonuclease activities involved in the maturation of double-strand breaks, gene conversion pathways can restore the original structure if a homologous sequence is available, such as a homologous chromosome in non-dividing somatic cells, or a sister chromatid after DNA replication (Molinier et al., (2004) Plant Cell 16:342-52). Ectopic and/or epigenic DNA sequences may also serve as a DNA repair template for homologous recombination (Puchta, (1999) Genetics 152:1173-81).

The donor DNA may be introduced by any means known in the art. The donor DNA may be provided by any transformation method known in the art including, for example, Agrobacterium-mediated transformation or biolistic particle bombardment. The donor DNA may be present transiently in the cell or it could be introduced via a viral replicon. In the presence of the Cas9 endonuclease and the target site, the donor DNA is inserted into the transformed plant's genome. (see guide language)

Further uses for guide RNA/Cas9 endonuclease systems have been described (See U.S. Patent Application US 2015-0082478 A1, published on Mar. 19, 2015, WO2015/026886 A1, published on Feb. 26, 2015, US 2015-0059010 A1, published on Feb. 26, 2015, U.S. application 62/023,246, filed on Jul. 7, 2014, and U.S. application 62/036,652, filed on Aug. 13, 2014, all of which are incorporated by reference herein) and include but are not limited to modifying or replacing nucleotide sequences of interest (such as a regulatory elements), insertion of polynucleotides of interest, gene knock-out, gene-knock in, modification of splicing sites and/or introducing alternate splicing sites, modifications of nucleotide sequences encoding a protein of interest, amino acid and/or protein fusions, and gene silencing by expressing an inverted repeat into a gene of interest.

Given the diversity of Type II CRISPR-Cas systems (Fonfara et al. (2014) Nucleic Acids Res. 42:2577-2590), it is plausible that many of the Cas9 endonucleases and cognate guide RNAs may have unique sequence recognition and enzymatic properties different from those previously described or characterized. For example, cleavage activity and specificity may be enhanced or proto-spacer adjacent motif (PAM) sequence may be different leading to increased genomic target site density. To tap into this vast unexplored diversity and expand the repertoire of Cas9 endonucleases and cognate guide RNAs available for genome targeting, the two components of Cas9 target site recognition, the PAM sequence and the guide RNA (either duplexed CRISPR RNA (crRNA) and trans-activating CRISPR RNA (tracrRNA) or chimeric fusion of crRNA and tracrRNA (single guide RNA (sgRNA), need to be established for each new system.

As described herein, CRISPR-Cas loci (including Cas9 genes and open reading frames, CRISPR array and anti-repeats) from uncharacterized CRISPR-Cas systems (FIGS. 1-23) were identified by searching internal Pioneer-DuPont databases consisting of microbial genomes. The Cas9 endonuclease described herein can be expressed and purified by methods known in the art (such as those described in Example 2 of U.S. patent application 62/162,377 filed May 15, 2015, incorporated herein by reference). As described herein (Example 1), the transcriptional direction of the tracrRNA for all the CRISPR-Cas systems can be deduced and examples of sgRNAs (SEQ ID NOs:) and its components (VT, crRNA repeat, loop, anti-repeat and 3′tracrRNA) were identified for each new diverse CRISPR-Cas endonuclease described herein.

Polynucleotides of interest are further described herein and include polynucleotides reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for genetic engineering will change accordingly.

Further provided are methods for identifying at least one plant cell, comprising in its genome, a polynucleotide of interest integrated at the target site. A variety of methods are available for identifying those plant cells with insertion into the genome at or near to the target site without using a screenable marker phenotype. Such methods can be viewed as directly analyzing a target sequence to detect any change in the target sequence, including but not limited to PCR methods, sequencing methods, nuclease digestion, Southern blots, and any combination thereof. See, for example, U.S. patent application Ser. No. 12/147,834, herein incorporated by reference to the extent necessary for the methods described herein. The method also comprises recovering a plant from the plant cell comprising a polynucleotide of Interest integrated into its genome. The plant may be sterile or fertile. It is recognized that any polynucleotide of interest can be provided, integrated into the plant genome at the target site, and expressed in a plant.

Polynucleotides/polypeptides of interest include, but are not limited to, herbicide-resistance coding sequences, insecticidal coding sequences, nematicidal coding sequences, antimicrobial coding sequences, antifungal coding sequences, antiviral coding sequences, abiotic and biotic stress tolerance coding sequences, or sequences modifying plant traits such as yield, grain quality, nutrient content, starch quality and quantity, nitrogen fixation and/or utilization, fatty acids, and oil content and/or composition. More specific polynucleotides of interest include, but are not limited to, genes that improve crop yield, polypeptides that improve desirability of crops, genes encoding proteins conferring resistance to abiotic stress, such as drought, nitrogen, temperature, salinity, toxic metals or trace elements, or those conferring resistance to toxins such as pesticides and herbicides, or to biotic stress, such as attacks by fungi, viruses, bacteria, insects, and nematodes, and development of diseases associated with these organisms. General categories of genes of interest include, for example, those genes involved in information, such as zinc fingers, those involved in communication, such as kinases, and those involved in housekeeping, such as heat shock proteins. More specific categories of transgenes, for example, include genes encoding important traits for agronomics, insect resistance, disease resistance, herbicide resistance, fertility or sterility, grain characteristics, and commercial products. Genes of interest include, generally, those involved in oil, starch, carbohydrate, or nutrient metabolism as well as those affecting kernel size, sucrose loading, and the like that can be stacked or used in combination with other traits, such as but not limited to herbicide resistance, described herein.

Agronomically important traits such as oil, starch, and protein content can be genetically altered in addition to using traditional breeding methods. Modifications include increasing content of oleic acid, saturated and unsaturated oils, increasing levels of lysine and sulfur, providing essential amino acids, and also modification of starch. Hordothionin protein modifications are described in U.S. Pat. Nos. 5,703,049, 5,885,801, 5,885,802, and 5,990,389, herein incorporated by reference.

Polynucleotide sequences of interest may encode proteins involved in providing disease or pest resistance. By “disease resistance” or “pest resistance” is intended that the plants avoid the harmful symptoms that are the outcome of the plant-pathogen interactions. Pest resistance genes may encode resistance to pests that have great yield drag such as rootworm, cutworm, European Corn Borer, and the like. Disease resistance and insect resistance genes such as lysozymes or cecropins for antibacterial protection, or proteins such as defensins, glucanases or chitinases for antifungal protection, or Bacillus thuringiensis endotoxins, protease inhibitors, collagenases, lectins, or glycosidases for controlling nematodes or insects are all examples of useful gene products. Genes encoding disease resistance traits include detoxification genes, such as against fumonisin (U.S. Pat. No. 5,792,931); avirulence (avr) and disease resistance (R) genes (Jones et al. (1994) Science 266:789; Martin et al. (1993) Science 262:1432; and Mindrinos et al. (1994) Cell 78:1089); and the like. Insect resistance genes may encode resistance to pests that have great yield drag such as rootworm, cutworm, European Corn Borer, and the like. Such genes include, for example, Bacillus thuringiensis toxic protein genes (U.S. Pat. Nos. 5,366,892; 5,747,450; 5,736,514; 5,723,756; 5,593,881; and Geiser et al. (1986) Gene 48:109); and the like.

An “herbicide resistance protein” or a protein resulting from expression of an “herbicide resistance-encoding nucleic acid molecule” includes proteins that confer upon a cell the ability to tolerate a higher concentration of an herbicide than cells that do not express the protein, or to tolerate a certain concentration of an herbicide for a longer period of time than cells that do not express the protein. Herbicide resistance traits may be introduced into plants by genes coding for resistance to herbicides that act to inhibit the action of acetolactate synthase (ALS), in particular the sulfonylurea-type herbicides, genes coding for resistance to herbicides that act to inhibit the action of glutamine synthase, such as phosphinothricin or basta (e.g., the bar gene), glyphosate (e.g., the EPSP synthase gene and the GAT gene), HPPD inhibitors (e.g, the HPPD gene) or other such genes known in the art. See, for example, U.S. Pat. Nos. 7,626,077, 5,310,667, 5,866,775, 6,225,114, 6,248,876, 7,169,970, 6,867,293, and U.S. Provisional Application No. 61/401,456, each of which is herein incorporated by reference. The bar gene encodes resistance to the herbicide basta, the nptII gene encodes resistance to the antibiotics kanamycin and geneticin, and the ALS-gene mutants encode resistance to the herbicide chlorsulfuron.

Furthermore, it is recognized that the polynucleotide of interest may also comprise antisense sequences complementary to at least a portion of the messenger RNA (mRNA) for a targeted gene sequence of interest. Antisense nucleotides are constructed to hybridize with the corresponding mRNA. Modifications of the antisense sequences may be made as long as the sequences hybridize to and interfere with expression of the corresponding mRNA. In this manner, antisense constructions having 70%, 80%, or 85% sequence identity to the corresponding antisense sequences may be used. Furthermore, portions of the antisense nucleotides may be used to disrupt the expression of the target gene. Generally, sequences of at least 50 nucleotides, 100 nucleotides, 200 nucleotides, or greater may be used.

In addition, the polynucleotide of interest may also be used in the sense orientation to suppress the expression of endogenous genes in plants. Methods for suppressing gene expression in plants using polynucleotides in the sense orientation are known in the art. The methods generally involve transforming plants with a DNA construct comprising a promoter that drives expression in a plant operably linked to at least a portion of a nucleotide sequence that corresponds to the transcript of the endogenous gene. Typically, such a nucleotide sequence has substantial sequence identity to the sequence of the transcript of the endogenous gene, generally greater than about 65% sequence identity, about 85% sequence identity, or greater than about 95% sequence identity. See, U.S. Pat. Nos. 5,283,184 and 5,034,323; herein incorporated by reference.

The polynucleotide of interest can also be a phenotypic marker. A phenotypic marker is screenable or a selectable marker that includes visual markers and selectable markers whether it is a positive or negative selectable marker. Any phenotypic marker can be used. Specifically, a selectable or screenable marker comprises a DNA segment that allows one to identify, or select for or against a molecule or a cell that contains it, often under particular conditions. These markers can encode an activity, such as, but not limited to, production of RNA, peptide, or protein, or can provide a binding site for RNA, peptides, proteins, inorganic and organic compounds or compositions and the like.

Examples of selectable markers include, but are not limited to, DNA segments that comprise restriction enzyme sites; DNA segments that encode products which provide resistance against otherwise toxic compounds including antibiotics, such as, spectinomycin, ampicillin, kanamycin, tetracycline, Basta, neomycin phosphotransferase II (NEO) and hygromycin phosphotransferase (HPT)); DNA segments that encode products which are otherwise lacking in the recipient cell (e.g., tRNA genes, auxotrophic markers); DNA segments that encode products which can be readily identified (e.g., phenotypic markers such β-galactosidase, GUS; fluorescent proteins such as green fluorescent protein (GFP), cyan (CFP), yellow (YFP), red (RFP), and cell surface proteins); the generation of new primer sites for PCR (e.g., the juxtaposition of two DNA sequence not previously juxtaposed), the inclusion of DNA sequences not acted upon or acted upon by a restriction endonuclease or other DNA modifying enzyme, chemical, etc.; and, the inclusion of a DNA sequences required for a specific modification (e.g., methylation) that allows its identification.

Additional selectable markers include genes that confer resistance to herbicidal compounds, such as glufosinate ammonium, bromoxynil, imidazolinones, and 2,4-dichlorophenoxyacetate (2,4-D). Commercial traits can also be encoded on a gene or genes that could increase for example, starch for ethanol production, or provide expression of proteins. Exogenous products include plant enzymes and products as well as those from other sources including prokaryotes and other eukaryotes. Such products include enzymes, cofactors, hormones, and the like. The level of proteins, particularly modified proteins having improved amino acid distribution to improve the nutrient value of the plant, can be increased. This is achieved by the expression of such proteins having enhanced amino acid content.

The transgenes, recombinant DNA molecules, DNA sequences of interest, and polynucleotides of interest can be comprise one or more DNA sequences for gene silencing. Methods for gene silencing involving the expression of DNA sequences in plant are known in the art include, but are not limited to, cosuppression, antisense suppression, double-stranded RNA (dsRNA) interference, hairpin RNA (hpRNA) interference, intron-containing hairpin RNA (ihpRNA) interference, transcriptional gene silencing, and micro RNA (miRNA) interference

As used herein, “nucleic acid” means a polynucleotide and includes a single or a double-stranded polymer of deoxyribonucleotide or ribonucleotide bases. Nucleic acids may also include fragments and modified nucleotides. Thus, the terms “polynucleotide”, “nucleic acid sequence”, “nucleotide sequence” and “nucleic acid fragment” are used interchangeably to denote a polymer of RNA and/or DNA that is single- or double-stranded, optionally containing synthetic, non-natural, or altered nucleotide bases. Nucleotides (usually found in their 5′-monophosphate form) are referred to by their single letter designation as follows: “A” for adenosine or deoxyadenosine (for RNA or DNA, respectively), “C” for cytosine or deoxycytosine, “G” for guanosine or deoxyguanosine, “U” for uridine, “T” for deoxythymidine, “R” for purines (A or G), “Y” for pyrimidines (C or T), “K” for G or T, “H” for A or C or T, “I” for inosine, and “N” for any nucleotide.

“Open reading frame” is abbreviated ORF.

The terms “subfragment that is functionally equivalent” and “functionally equivalent subfragment” are used interchangeably herein. These terms refer to a portion or subsequence of an isolated nucleic acid fragment in which the ability to alter gene expression or produce a certain phenotype is retained whether or not the fragment or subfragment encodes an active enzyme. For example, the fragment or subfragment can be used in the design of genes to produce the desired phenotype in a transformed plant. Genes can be designed for use in suppression by linking a nucleic acid fragment or subfragment thereof, whether or not it encodes an active enzyme, in the sense or antisense orientation relative to a plant promoter sequence.

The term “conserved domain” or “motif” means a set of amino acids conserved at specific positions along an aligned sequence of evolutionarily related proteins. While amino acids at other positions can vary between homologous proteins, amino acids that are highly conserved at specific positions indicate amino acids that are essential to the structure, the stability, or the activity of a protein. Because they are identified by their high degree of conservation in aligned sequences of a family of protein homologues, they can be used as identifiers, or “signatures”, to determine if a protein with a newly determined sequence belongs to a previously identified protein family.

Polynucleotide and polypeptide sequences, variants thereof, and the structural relationships of these sequences can be described by the terms “homology”, “homologous”, “substantially identical”, “substantially similar” and “corresponding substantially” which are used interchangeably herein. These refer to polypeptide or nucleic acid fragments wherein changes in one or more amino acids or nucleotide bases do not affect the function of the molecule, such as the ability to mediate gene expression or to produce a certain phenotype. These terms also refer to modification(s) of nucleic acid fragments that do not substantially alter the functional properties of the resulting nucleic acid fragment relative to the initial, unmodified fragment. These modifications include deletion, substitution, and/or insertion of one or more nucleotides in the nucleic acid fragment.

Substantially similar nucleic acid sequences encompassed may be defined by their ability to hybridize (under moderately stringent conditions, e.g., 0.5×SSC, 0.1% SDS, 60° C.) with the sequences exemplified herein, or to any portion of the nucleotide sequences disclosed herein and which are functionally equivalent to any of the nucleic acid sequences disclosed herein. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 80% sequence identity, or 90% sequence identity, up to and including 100% sequence identity (i.e., fully complementary) with each other.

The term “stringent conditions” or “stringent hybridization conditions” includes reference to conditions under which a probe will selectively hybridize to its target sequence in an in vitro hybridization assay. Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length.

Typically, stringent conditions will be those in which the salt concentration is less than about 1.5 M Na ion, typically about 0.01 to 1.0 M Na ion concentration (or other salt(s)) at pH 7.0 to 8.3, and at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

“Sequence identity” or “identity” in the context of nucleic acid or polypeptide sequences refers to the nucleic acid bases or amino acid residues in two sequences that are the same when aligned for maximum correspondence over a specified comparison window.

The term “percentage of sequence identity” refers to the value determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. Useful examples of percent sequence identities include, but are not limited to, 50%, 55%, 60%, 65%, 70%, 75%, 80%, 85%, 90% or 95%, or any integer percentage from 50% to 100%. These identities can be determined using any of the programs described herein.

Sequence alignments and percent identity or similarity calculations may be determined using a variety of comparison methods designed to detect homologous sequences including, but not limited to, the MegAlign™ program of the LASERGENE bioinformatics computing suite (DNASTAR Inc., Madison, Wis.). Within the context of this application it will be understood that where sequence analysis software is used for analysis, that the results of the analysis will be based on the “default values” of the program referenced, unless otherwise specified. As used herein “default values” will mean any set of values or parameters that originally load with the software when first initialized.

The “Clustal V method of alignment” corresponds to the alignment method labeled Clustal V (described by Higgins and Sharp, (1989) CABIOS 5:151-153; Higgins et al., (1992) Comput Appl Biosci 8:189-191) and found in the MegAlign™ program of the LASERGENE bioinformatics computing suite (DNASTAR Inc., Madison, Wis.). For multiple alignments, the default values correspond to GAP PENALTY=10 and GAP LENGTH PENALTY=10. Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences using the Clustal V program, it is possible to obtain a “percent identity” by viewing the “sequence distances” table in the same program.

The “Clustal W method of alignment” corresponds to the alignment method labeled Clustal W (described by Higgins and Sharp, (1989) CABIOS 5:151-153; Higgins et al., (1992) Comput Appl Biosci 8:189-191) and found in the MegAlign™ v6.1 program of the LASERGENE bioinformatics computing suite (DNASTAR Inc., Madison, Wis.). Default parameters for multiple alignment (GAP PENALTY=10, GAP LENGTH PENALTY=0.2, Delay Divergen Seqs (%)=30, DNA Transition Weight=0.5, Protein Weight Matrix=Gonnet Series, DNA Weight Matrix=IUB). After alignment of the sequences using the Clustal W program, it is possible to obtain a “percent identity” by viewing the “sequence distances” table in the same program.

Unless otherwise stated, sequence identity/similarity values provided herein refer to the value obtained using GAP Version 10 (GCG, Accelrys, San Diego, Calif.) using the following parameters: % identity and % similarity for a nucleotide sequence using a gap creation penalty weight of 50 and a gap length extension penalty weight of 3, and the nwsgapdna.cmp scoring matrix; % identity and % similarity for an amino acid sequence using a GAP creation penalty weight of 8 and a gap length extension penalty of 2, and the BLOSUM62 scoring matrix (Henikoff and Henikoff, (1989) Proc. Natl. Acad. Sci. USA 89:10915). GAP uses the algorithm of Needleman and Wunsch, (1970) J Mol Biol 48:443-53, to find an alignment of two complete sequences that maximizes the number of matches and minimizes the number of gaps. GAP considers all possible alignments and gap positions and creates the alignment with the largest number of matched bases and the fewest gaps, using a gap creation penalty and a gap extension penalty in units of matched bases.

“BLAST” is a searching algorithm provided by the National Center for Biotechnology Information (NCBI) used to find regions of similarity between biological sequences. The program compares nucleotide or protein sequences to sequence databases and calculates the statistical significance of matches to identify sequences having sufficient similarity to a query sequence such that the similarity would not be predicted to have occurred randomly. BLAST reports the identified sequences and their local alignment to the query sequence.

It is well understood by one skilled in the art that many levels of sequence identity are useful in identifying polypeptides from other species or modified naturally or synthetically wherein such polypeptides have the same or similar function or activity. Useful examples of percent identities include, but are not limited to, 50%, 55%, 60%, 65%, 70%, 75%, 80%, 85%, 90% or 95%, or any integer percentage from 50% to 100%. Indeed, any integer amino acid identity from 50% to 100% may be useful in describing the present disclosure, such as 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%.

“Gene” includes a nucleic acid fragment that expresses a functional molecule such as, but not limited to, a specific protein, including regulatory sequences preceding (5′ non-coding sequences) and following (3′ non-coding sequences) the coding sequence. “Native gene” refers to a gene as found in nature with its own regulatory sequences.

A “mutated gene” is a gene that has been altered through human intervention. Such a “mutated gene” has a sequence that differs from the sequence of the corresponding non-mutated gene by at least one nucleotide addition, deletion, or substitution. In certain embodiments of the disclosure, the mutated gene comprises an alteration that results from a guide polynucleotide/Cas9 endonuclease system as disclosed herein. A mutated plant is a plant comprising a mutated gene.

As used herein, a “targeted mutation” is a mutation in a gene, such as a native gene, that was made by altering a target sequence within that gene using a method involving a double-strand-break-inducing agent that is capable of inducing a double-strand break in the DNA of the target sequence as disclosed herein or known in the art.

The guide RNA/Cas9 endonuclease induced targeted mutation can occur in a nucleotide sequence that is located within or outside a genomic target site that is recognized and cleaved by a Cas9 endonuclease.

The term “genome” as it applies to a plant cells encompasses not only chromosomal DNA found within the nucleus, but organelle DNA found within subcellular components (e.g., mitochondria, or plastid) of the cell.

A “codon-modified gene” or “codon-preferred gene” or “codon-optimized gene” is a gene having its frequency of codon usage designed to mimic the frequency of preferred codon usage of the host cell.

An “allele” is one of several alternative forms of a gene occupying a given locus on a chromosome. When all the alleles present at a given locus on a chromosome are the same, that plant is homozygous at that locus. If the alleles present at a given locus on a chromosome differ, that plant is heterozygous at that locus.

“Coding sequence” refers to a polynucleotide sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to: promoters, translation leader sequences, 5′ untranslated sequences, 3′ untranslated sequences, introns, polyadenylation target sequences, RNA processing sites, effector binding sites, and stem-loop structures.

Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference. Additional sequence modifications are known to enhance gene expression in a plant host. These include, for example, elimination of: one or more sequences encoding spurious polyadenylation signals, one or more exon-intron splice site signals, one or more transposon-like repeats, and other such well-characterized sequences that may be deleterious to gene expression. The G-C content of the sequence may be adjusted to levels average for a given plant host, as calculated by reference to known genes expressed in the host plant cell. When possible, the sequence is modified to avoid one or more predicted hairpin secondary mRNA structures. Thus, “a plant-optimized nucleotide sequence” of the present disclosure comprises one or more of such sequence modifications.

A promoter is a region of DNA involved in recognition and binding of RNA polymerase and other proteins to initiate transcription. The promoter sequence consists of proximal and more distal upstream elements, the latter elements often referred to as enhancers. An “enhancer” is a DNA sequence that can stimulate promoter activity, and may be an innate element of the promoter or a heterologous element inserted to enhance the level or tissue-specificity of a promoter. Promoters may be derived in their entirety from a native gene, or be composed of different elements derived from different promoters found in nature, and/or comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity. Promoters that cause a gene to be expressed in most cell types at most times are commonly referred to as “constitutive promoters”.

It has been shown that certain promoters are able to direct RNA synthesis at a higher rate than others. These are called “strong promoters”. Certain other promoters have been shown to direct RNA synthesis at higher levels only in particular types of cells or tissues and are often referred to as “tissue specific promoters”, or “tissue-preferred promoters” if the promoters direct RNA synthesis preferably in certain tissues but also in other tissues at reduced levels. Since patterns of expression of a chimeric gene (or genes) introduced into a plant are controlled using promoters, there is an ongoing interest in the isolation of novel promoters which are capable of controlling the expression of a chimeric gene or (genes) at certain levels in specific tissue types or at specific plant developmental stages.

A “chimeric gene” refers to any gene that is not a native gene, comprising regulatory and coding sequences that are not found together in nature (i.e., the regulatory and coding regions are heterologous with each other). Accordingly, a chimeric gene may comprise regulatory sequences and coding sequences that are derived from different sources, or regulatory sequences and coding sequences derived from the same source, but arranged in a manner different than that found in nature. A “foreign” or “heterologous” gene refers to a gene that is introduced into the host organism by gene transfer. Foreign genes can comprise native genes inserted into a non-native organism, native genes introduced into a new location within the native host, or chimeric genes. The polynucleotide sequences in certain embodiments disclosed herein are heterologous. A “transgene” is a gene that has been introduced into the genome by a transformation procedure. A “codon-optimized” open reading frame has its frequency of codon usage designed to mimic the frequency of preferred codon usage of the host cell. A plant promoter can include a promoter capable of initiating transcription in a plant cell, for a review of plant promoters, see, Potenza et al., (2004) In Vitro Cell Dev Biol 40:1-22. Constitutive promoters include, for example, the core promoter of the Rsyn7 promoter and other constitutive promoters disclosed in WO99/43838 and U.S. Pat. No. 6,072,050; the core CaMV 35S promoter (Odell et al., (1985) Nature 313:810-2); rice actin (McElroy et al., (1990) Plant Cell 2:163-71); ubiquitin (Christensen et al., (1989) Plant Mol Biol 12:619-32; Christensen et al., (1992) Plant Mol Biol 18:675-89); pEMU (Last et al., (1991) Theor Appl Genet 81:581-8); MAS (Velten et al., (1984) EMBO J3:2723-30); ALS promoter (U.S. Pat. No. 5,659,026), and the like. Other constitutive promoters are described in, for example, U.S. Pat. Nos. 5,608,149; 5,608,144; 5,604,121; 5,569,597; 5,466,785; 5,399,680; 5,268,463; 5,608,142 and 6,177,611. In some examples an inducible promoter may be used. Pathogen-inducible promoters induced following infection by a pathogen include, but are not limited to those regulating expression of PR proteins, SAR proteins, beta-1,3-glucanase, chitinase, etc.

Chemical-regulated promoters can be used to modulate the expression of a gene in a plant through the application of an exogenous chemical regulator. The promoter may be a chemical-inducible promoter, where application of the chemical induces gene expression, or a chemical-repressible promoter, where application of the chemical represses gene expression. Chemical-inducible promoters include, but are not limited to, the maize 1n2-2 promoter, activated by benzene sulfonamide herbicide safeners (De Veylder et al., (1997) Plant Cell Physiol 38:568-77), the maize GST promoter (GST-II-27, WO93/01294), activated by hydrophobic electrophilic compounds used as pre-emergent herbicides, and the tobacco PR-1a promoter (Ono et al., (2004) Biosci Biotechnol Biochem 68:803-7) activated by salicylic acid. Other chemical-regulated promoters include steroid-responsive promoters (see, for example, the glucocorticoid-inducible promoter (Schena et al., (1991) Proc. Natl. Acad. Sci. USA 88:10421-5; McNellis et al., (1998) Plant J 14:247-257); tetracycline-inducible and tetracycline-repressible promoters (Gatz et al., (1991) Mol Gen Genet 227:229-37; U.S. Pat. Nos. 5,814,618 and 5,789,156).

Tissue-preferred promoters can be utilized to target enhanced expression within a particular plant tissue. Tissue-preferred promoters include, for example, Kawamata et al., (1997) Plant Cell Physiol 38:792-803; Hansen et al., (1997) Mol Gen Genet 254:337-43; Russell et al., (1997) Transgenic Res 6:157-68; Rinehart et al., (1996) Plant Physiol 112:1331-41; Van Camp et al., (1996) Plant Physiol 112:525-35; Canevascini et al., (1996) Plant Physiol 112:513-524; Lam, (1994) Results Probl Cell Differ 20:181-96; and Guevara-Garcia et al., (1993) Plant J 4:495-505. Leaf-preferred promoters include, for example, Yamamoto et al., (1997) Plant J 12:255-65; Kwon et al., (1994) Plant Physiol 105:357-67; Yamamoto et al., (1994) Plant Cell Physiol 35:773-8; Gotor et al., (1993) Plant J 3:509-18; Orozco et al., (1993) Plant Mol Biol 23:1129-38; Matsuoka et al., (1993) Proc. Natl. Acad. Sci. USA 90:9586-90; Simpson et al., (1958) EMBO J 4:2723-9; Timko et al., (1988) Nature 318:57-8. Root-preferred promoters include, for example, Hire et al., (1992) Plant Mol Biol 20:207-18 (soybean root-specific glutamine synthase gene); Miao et al., (1991) Plant Cell 3:11-22 (cytosolic glutamine synthase (GS)); Keller and Baumgartner, (1991) Plant Cell 3:1051-61 (root-specific control element in the GRP 1.8 gene of French bean); Sanger et al., (1990) Plant Mol Biol 14:433-43 (root-specific promoter of A. tumefaciens mannopine synthase (MAS)); Bogusz et al., (1990) Plant Cell 2:633-41 (root-specific promoters isolated from Parasponia andersonii and Trema tomentosa); Leach and Aoyagi, (1991) Plant Sci 79:69-76 (A. rhizogenes roIC and roID root-inducing genes); Teeri et al., (1989) EMBO J 8:343-50 (Agrobacterium wound-induced TR1′ and TR2′ genes); VfENOD-GRP3 gene promoter (Kuster et al., (1995) Plant Mol Biol 29:759-72); and roIB promoter (Capana et al., (1994) Plant Mol Biol 25:681-91; phaseolin gene (Murai et al., (1983) Science 23:476-82; Sengopta-Gopalen et al., (1988) Proc. Natl. Acad. Sci. USA 82:3320-4). See also, U.S. Pat. Nos. 5,837,876; 5,750,386; 5,633,363; 5,459,252; 5,401,836; 5,110,732 and 5,023,179.

Seed-preferred promoters include both seed-specific promoters active during seed development, as well as seed-germinating promoters active during seed germination. See, Thompson et al., (1989) BioEssays 10:108. Seed-preferred promoters include, but are not limited to, Cim1 (cytokinin-induced message); cZ19B1 (maize 19 kDa zein); and milps (myo-inositol-1-phosphate synthase); (WO00/11177; and U.S. Pat. No. 6,225,529). For dicots, seed-preferred promoters include, but are not limited to, bean β-phaseolin, napin, β-conglycinin, soybean lectin, cruciferin, and the like. For monocots, seed-preferred promoters include, but are not limited to, maize 15 kDa zein, 22 kDa zein, 27 kDa gamma zein, waxy, shrunken 1, shrunken 2, globulin 1, oleosin, and nuc1. See also, WO00/12733, where seed-preferred promoters from END1 and END2 genes are disclosed.

The term “inducible promoter” refers to promoters that selectively express a coding sequence or functional RNA in response to the presence of an endogenous or exogenous stimulus, for example by chemical compounds (chemical inducers) or in response to environmental, hormonal, chemical, and/or developmental signals. Inducible or regulated promoters include, for example, promoters induced or regulated by light, heat, stress, flooding or drought, salt stress, osmotic stress, phytohormones, wounding, or chemicals such as ethanol, abscisic acid (ABA), jasmonate, salicylic acid, or safeners.

An example of a stress-inducible is RD29A promoter (Kasuga et al. (1999) Nature Biotechnol. 17:287-91). One of ordinary skill in the art is familiar with protocols for simulating drought conditions and for evaluating drought tolerance of plants that have been subjected to simulated or naturally-occurring drought conditions. For example, one can simulate drought conditions by giving plants less water than normally required or no water over a period of time, and one can evaluate drought tolerance by looking for differences in physiological and/or physical condition, including (but not limited to) vigor, growth, size, or root length, or in particular, leaf color or leaf area size. Other techniques for evaluating drought tolerance include measuring chlorophyll fluorescence, photosynthetic rates and gas exchange rates. Also, one of ordinary skill in the art is familiar with protocols for simulating stress conditions such as osmotic stress, salt stress and temperature stress and for evaluating stress tolerance of plants that have been subjected to simulated or naturally-occurring stress conditions.

Another example of an inducible promoter useful in plant cells has been described in US patent application, US 2013-0312137A1, published on Nov. 21, 2013, incorporated by reference herein. US patent application US 2013-0312137A1 describes a ZmCAS1 promoter from a CBSU-Anther_Subtraction library (CAS1) gene encoding a mannitol dehydrogenase from maize, and functional fragments thereof. The ZmCAS1 promoter (also referred to as “CAS1 promoter”, “mannitol dehydrogenase promoter”, “mdh promoter”) can be induced by a chemical or stress treatment. The chemical can be a safener such as, but not limited to, N-(aminocarbonyl)-2-chlorobenzenesulfonamide (2-CBSU). The stress treatment can be a heat treatment such as, but not limited to, a heat shock treatment (see also U.S. provisional patent application,62/120,421, filed on Feb. 25, 2015, and incorporated by reference herein.

New promoters of various types useful in plant cells are constantly being discovered; numerous examples may be found in the compilation by Okamuro and Goldberg, (1989) In The Biochemistry of Plants, Vol. 115, Stumpf and Conn, eds (New York, N.Y.: Academic Press), pp. 1-82.

“Translation leader sequence” refers to a polynucleotide sequence located between the promoter sequence of a gene and the coding sequence. The translation leader sequence is present in the mRNA upstream of the translation start sequence. The translation leader sequence may affect processing of the primary transcript to mRNA, mRNA stability or translation efficiency. Examples of translation leader sequences have been described (e.g., Turner and Foster, (1995) Mol Biotechnol 3:225-236).

“3′ non-coding sequences”, “transcription terminator” or “termination sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et aL, (1989) Plant Cell 1:671-680.

“RNA transcript” refers to the product resulting from RNA polymerase-catalyzed transcription of a DNA sequence. When the RNA transcript is a perfect complimentary copy of the DNA sequence, it is referred to as the primary transcript or pre-mRNA. A RNA transcript is referred to as the mature RNA or mRNA when it is a RNA sequence derived from post-transcriptional processing of the primary transcript pre mRNA. “Messenger RNA” or “mRNA” refers to the RNA that is without introns and that can be translated into protein by the cell. “cDNA” refers to a DNA that is complementary to, and synthesized from, an mRNA template using the enzyme reverse transcriptase. The cDNA can be single-stranded or converted into double-stranded form using the Klenow fragment of DNA polymerase I. “Sense” RNA refers to RNA transcript that includes the mRNA and can be translated into protein within a cell or in vitro. “Antisense RNA” refers to an RNA transcript that is complementary to all or part of a target primary transcript or mRNA, and that blocks the expression of a target gene (see, e.g., U.S. Pat. No. 5,107,065). The complementarity of an antisense RNA may be with any part of the specific gene transcript, i.e., at the 5′ non-coding sequence, 3′ non-coding sequence, introns, or the coding sequence. “Functional RNA” refers to antisense RNA, ribozyme RNA, or other RNA that may not be translated but yet has an effect on cellular processes. The terms “complement” and “reverse complement” are used interchangeably herein with respect to mRNA transcripts, and are meant to define the antisense RNA of the message.

The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., the coding sequence is under the transcriptional control of the promoter). Coding sequences can be operably linked to regulatory sequences in a sense or antisense orientation. In another example, the complementary RNA regions can be operably linked, either directly or indirectly, 5′ to the target mRNA, or 3′ to the target mRNA, or within the target mRNA, or a first complementary region is 5′ and its complement is 3′ to the target mRNA.

Standard recombinant DNA and molecular cloning techniques used herein are well known in the art and are described more fully in Sambrook et al., Molecular Cloning: A Laboratory Manual; Cold Spring Harbor Laboratory: Cold Spring Harbor, N.Y. (1989). Transformation methods are well known to those skilled in the art and are described infra.

The term “recombinant” refers to an artificial combination of two otherwise separated segments of sequence, e.g., by chemical synthesis, or manipulation of isolated segments of nucleic acids by genetic engineering techniques.

The terms “plasmid”, “vector” and “cassette” refer to an extra chromosomal element often carrying genes that are not part of the central metabolism of the cell, and usually in the form of double-stranded DNA. Such elements may be autonomously replicating sequences, genome integrating sequences, phage, or nucleotide sequences, in linear or circular form, of a single- or double-stranded DNA or RNA, derived from any source, in which a number of nucleotide sequences have been joined or recombined into a unique construction which is capable of introducing a polynucleotide of interest into a cell. “Transformation cassette” refers to a specific vector containing a gene and having elements in addition to the gene that facilitates transformation of a particular host cell. “Expression cassette” refers to a specific vector containing a gene and having elements in addition to the gene that allow for expression of that gene in a host.

The terms “recombinant DNA molecule”, “recombinant construct”, “expression construct”, “ construct”, “construct”, and “recombinant DNA construct” are used interchangeably herein. A recombinant construct comprises an artificial combination of nucleic acid fragments, e.g., regulatory and coding sequences that are not all found together in nature. For example, a construct may comprise regulatory sequences and coding sequences that are derived from different sources, or regulatory sequences and coding sequences derived from the same source, but arranged in a manner different than that found in nature. Such a construct may be used by itself or may be used in conjunction with a vector. If a vector is used, then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells. The skilled artisan will also recognize that different independent transformation events may result in different levels and patterns of expression (Jones et al., (1985) EMBO J 4:2411-2418; De Almeida et aL, (1989) Mol Gen Genetics 218:78-86), and thus that multiple events are typically screened in order to obtain lines displaying the desired expression level and pattern. Such screening may be accomplished standard molecular biological, biochemical, and other assays including Southern analysis of DNA, Northern analysis of mRNA expression, PCR, real time quantitative PCR (qPCR), reverse transcription PCR (RT-PCR), immunoblotting analysis of protein expression, enzyme or activity assays, and/or phenotypic analysis.

The term “expression”, as used herein, refers to the production of a functional end-product (e.g., an mRNA, guide RNA, or a protein) in either precursor or mature form.

The term “introducing” includes providing a nucleic acid (e.g., expression construct) or protein into a cell. “Introducing” is intended to mean presenting to the organism, such as a cell or organism, the polynucleotide or polypeptide or polynucleotide-protein complex (also referred to as ribonucleotide protein complex or RNP), in such a manner that the component(s) gains access to the interior of a cell of the organism or to the cell itself. The methods and compositions do not depend on a particular method for introducing a sequence into an organism or cell, only that the polynucleotide or polypeptide gains access to the interior of at least one cell of the organism. Introducing includes reference to the incorporation of a nucleic acid into a eukaryotic or prokaryotic cell where the nucleic acid may be incorporated into the genome of the cell, and includes reference to the transient (direct) provision of a nucleic acid, protein or polynucleotide-protein complex (PGEN, RGEN) to the cell.

Introduced includes reference to stable or transient transformation methods, as well as sexually crossing. Thus, “introducing” in the context of inserting a nucleic acid fragment (e.g., a recombinant DNA construct/expression construct) into a cell, includes “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid, or mitochondrial DNA), converted into an autonomous replicon, or transiently expressed (e.g., transfected mRNA).

“Mature” protein refers to a post-translationally processed polypeptide (i.e., one from which any pre- or propeptides present in the primary translation product have been removed). “Precursor” protein refers to the primary product of translation of mRNA (i.e., with pre- and propeptides still present). Pre- and propeptides may be but are not limited to intracellular localization signals.

“Stable transformation” refers to the transfer of a nucleic acid fragment into a genome of a host organism, including both nuclear and organellar genomes, resulting in genetically stable inheritance. In contrast, “transient transformation” refers to the transfer of a nucleic acid fragment into the nucleus, or other DNA-containing organelle, of a host organism resulting in gene expression without integration or stable inheritance. Host organisms containing the transformed nucleic acid fragments are referred to as “transgenic” organisms.

The commercial development of genetically improved germplasm has also advanced to the stage of introducing multiple traits into crop plants, often referred to as a gene stacking approach. In this approach, multiple genes conferring different characteristics of interest can be introduced into a plant. Gene stacking can be accomplished by many means including but not limited to co-transformation, retransformation, and crossing lines with different genes of interest.

The term “plant” refers to whole plants, plant organs, plant tissues, seeds, plant cells, seeds and progeny of the same. Plant cells include, without limitation, cells from seeds, suspension cultures, embryos, meristematic regions, callus tissue, leaves, roots, shoots, gametophytes, sporophytes, pollen and microspores. Plant parts include differentiated and undifferentiated tissues including, but not limited to roots, stems, shoots, leaves, pollens, seeds, tumor tissue and various forms of cells and culture (e.g., single cells, protoplasts, embryos, and callus tissue). The plant tissue may be in plant or in a plant organ, tissue or cell culture. The term “plant organ” refers to plant tissue or a group of tissues that constitute a morphologically and functionally distinct part of a plant. The term “genome” refers to the entire complement of genetic material (genes and non-coding sequences) that is present in each cell of an organism, or virus or organelle; and/or a complete set of chromosomes inherited as a (haploid) unit from one parent. “Progeny” comprises any subsequent generation of a plant.

A transgenic plant includes, for example, a plant which comprises within its genome a heterologous polynucleotide introduced by a transformation step. The heterologous polynucleotide can be stably integrated within the genome such that the polynucleotide is passed on to successive generations. The heterologous polynucleotide may be integrated into the genome alone or as part of a recombinant DNA construct. A transgenic plant can also comprise more than one heterologous polynucleotide within its genome. Each heterologous polynucleotide may confer a different trait to the transgenic plant. A heterologous polynucleotide can include a sequence that originates from a foreign species, or, if from the same species, can be substantially modified from its native form. Transgenic can include any cell, cell line, callus, tissue, plant part or plant, the genotype of which has been altered by the presence of heterologous nucleic acid including those transgenics initially so altered as well as those created by sexual crosses or asexual propagation from the initial transgenic. The alterations of the genome (chromosomal or extra-chromosomal) by conventional plant breeding methods, by the genome editing procedure described herein that does not result in an insertion of a foreign polynucleotide, or by naturally occurring events such as random cross-fertilization, non-recombinant viral infection, non-recombinant bacterial transformation, non-recombinant transposition, or spontaneous mutation are not intended to be regarded as transgenic.

In certain embodiments of the disclosure, a fertile plant is a plant that produces viable male and female gametes and is self-fertile. Such a self-fertile plant can produce a progeny plant without the contribution from any other plant of a gamete and the genetic material contained therein. Other embodiments of the disclosure can involve the use of a plant that is not self-fertile because the plant does not produce male gametes, or female gametes, or both, that are viable or otherwise capable of fertilization. As used herein, a “male sterile plant” is a plant that does not produce male gametes that are viable or otherwise capable of fertilization. As used herein, a “female sterile plant” is a plant that does not produce female gametes that are viable or otherwise capable of fertilization. It is recognized that male-sterile and female-sterile plants can be female-fertile and male-fertile, respectively. It is further recognized that a male fertile (but female sterile) plant can produce viable progeny when crossed with a female fertile plant and that a female fertile (but male sterile) plant can produce viable progeny when crossed with a male fertile plant.

The guided Cas9 endonuclease systems of the present disclosure can be used in any prokaryotic or eukaryotic organism including non-conventional yeast and yeast or any fungal species that predominantly exist in unicellular form.

A “centimorgan” (cM) or “map unit” is the distance between two linked genes, markers, target sites, loci, or any pair thereof, wherein 1% of the products of meiosis are recombinant. Thus, a centimorgan is equivalent to a distance equal to a 1% average recombination frequency between the two linked genes, markers, target sites, loci, or any pair thereof.

The present disclosure finds use in the breeding of plants comprising one or more transgenic traits. Most commonly, transgenic traits are randomly inserted throughout the plant genome as a consequence of transformation systems based on Agrobacterium, biolistics, or other commonly used procedures. More recently, gene targeting protocols have been developed that enable directed transgene insertion. One important technology, site-specific integration (SSI) enables the targeting of a transgene to the same chromosomal location as a previously inserted transgene. Custom-designed meganucleases and custom-designed zinc finger meganucleases allow researchers to design nucleases to target specific chromosomal locations, and these reagents allow the targeting of transgenes at the chromosomal site cleaved by these nucleases.

The currently used systems for precision genetic engineering of eukaryotic genomes, e.g. plant genomes, rely upon homing endonucleases, meganucleases, zinc finger nucleases, and transcription activator—like effector nucleases (TALENs), which require de novo protein engineering for every new target locus. The highly specific, RNA-directed DNA nuclease, guide RNA/ Cas9 endonuclease system described herein, is more easily customizable and therefore more useful when modification of many different target sequences is the goal. This disclosure takes further advantage of the two component nature of the guide RNA/ Cas9 system, with its constant protein component, the Cas9 endonuclease, and its variable and easily reprogrammable targeting component, the guide RNA or the crRNA.

The guide RNA/Cas9 system described herein is especially useful for genome engineering, especially plant genome engineering, in circumstances where nuclease off-target cutting can be toxic to the targeted cells. In one embodiment of the guide RNA/Cas9 system described herein, the constant component, in the form of an expression-optimized Cas9 gene, is stably integrated into the target genome, e.g. plant genome. Expression of the Cas9 gene is under control of a promoter, e.g. plant promoter, which can be a constitutive promoter, tissue-specific promoter or inducible promoter, e.g. temperature-inducible, stress-inducible, developmental stage inducible, or chemically inducible promoter. In the absence of the variable component, i.e. the guide RNA or crRNA, the Cas9 protein is not able to cut DNA and therefore its presence in the plant cell should have little or no consequence. Hence a key advantage of the guide RNA/Cas9 system described herein is the ability to create and maintain a cell line or transgenic organism capable of efficient expression of the Cas9 protein with little or no consequence to cell viability. In order to induce cutting at desired genomic sites to achieve targeted genetic modifications, guide RNAs or crRNAs can be introduced by a variety of methods into cells containing the stably-integrated and expressed cas9 gene. For example, guide RNAs or crRNAs can be chemically or enzymatically synthesized, and introduced into the Cas9 expressing cells via direct delivery methods such a particle bombardment or electroporation.

Alternatively, genes capable of efficiently expressing guide RNAs or crRNAs in the target cells can be synthesized chemically, enzymatically or in a biological system, and these genes can be introduced into the Cas9 expressing cells via direct delivery methods such a particle bombardment, electroporation or biological delivery methods such as Agrobacterium mediated DNA delivery.

A guide RNA/Cas9 system mediating gene targeting can be used in methods for directing transgene insertion and/or for producing complex transgenic trait loci comprising multiple transgenes in a fashion similar as disclosed in WO2013/0198888 (published Aug. 1, 2013) where instead of using a double strand break inducing agent to introduce a gene of interest, a guide RNA/Cas9 system as disclosed herein is used. In one embodiment, a complex transgenic trait locus is a genomic locus that has multiple transgenes genetically linked to each other. By inserting independent transgenes within 0.1, 0.2, 0.3, 0.4, 0.5, 1.0, 2, or even 5 centimorgans (cM) from each other, the transgenes can be bred as a single genetic locus (see, for example, U.S. patent application Ser. No. 13/427,138) or PCT application PCT/US2012/030061. After selecting a plant comprising a transgene, plants containing (at least) one transgenes can be crossed to form an F1 that contains both transgenes. In progeny from these F1 (F2 or BC1) 1/500 progeny would have the two different transgenes recombined onto the same chromosome. The complex locus can then be bred as single genetic locus with both transgene traits. This process can be repeated to stack as many traits as desired.

Chromosomal intervals that correlate with a phenotype or trait of interest can be identified. A variety of methods well known in the art are available for identifying chromosomal intervals. The boundaries of such chromosomal intervals are drawn to encompass markers that will be linked to the gene controlling the trait of interest. In other words, the chromosomal interval is drawn such that any marker that lies within that interval (including the terminal markers that define the boundaries of the interval) can be used as a marker for northern leaf blight resistance. In one embodiment, the chromosomal interval comprises at least one QTL, and furthermore, may indeed comprise more than one QTL. Close proximity of multiple QTLs in the same interval may obfuscate the correlation of a particular marker with a particular QTL, as one marker may demonstrate linkage to more than one QTL. Conversely, e.g., if two markers in close proximity show co-segregation with the desired phenotypic trait, it is sometimes unclear if each of those markers identifies the same QTL or two different QTL. The term “quantitative trait locus” or “QTL” refers to a region of DNA that is associated with the differential expression of a quantitative phenotypic trait in at least one genetic background, e.g., in at least one breeding population. The region of the QTL encompasses or is closely linked to the gene or genes that affect the trait in question. An “allele of a QTL” can comprise multiple genes or other genetic factors within a contiguous genomic region or linkage group, such as a haplotype. An allele of a QTL can denote a haplotype within a specified window wherein said window is a contiguous genomic region that can be defined, and tracked, with a set of one or more polymorphic markers. A haplotype can be defined by the unique fingerprint of alleles at each marker within the specified window.

A variety of methods are available to identify those cells having an altered genome at or near a target site without using a screenable marker phenotype. Such methods can be viewed as directly analyzing a target sequence to detect any change in the target sequence, including but not limited to PCR methods, sequencing methods, nuclease digestion, Southern blots, and any combination thereof.

Proteins may be altered in various ways including amino acid substitutions, deletions, truncations, and insertions. Methods for such manipulations are generally known. For example, amino acid sequence variants of the protein(s) can be prepared by mutations in the DNA. Methods for mutagenesis and nucleotide sequence alterations include, for example, Kunkel, (1985) Proc. Natl. Acad. Sci. USA 82:488-92; Kunkel et al., (1987) Meth Enzymol 154:367-82; U.S. Pat. No. 4,873,192; Walker and Gaastra, eds. (1983) Techniques in Molecular Biology (MacMillan Publishing Company, New York) and the references cited therein. Guidance regarding amino acid substitutions not likely to affect biological activity of the protein is found, for example, in the model of Dayhoff et al., (1978) Atlas of Protein Sequence and Structure (Natl Biomed Res Found, Washington, D.C.). Conservative substitutions, such as exchanging one amino acid with another having similar properties, may be preferable. Conservative deletions, insertions, and amino acid substitutions are not expected to produce radical changes in the characteristics of the protein, and the effect of any substitution, deletion, insertion, or combination thereof can be evaluated by routine screening assays. Assays for double-strand-break-inducing activity are known and generally measure the overall activity and specificity of the agent on DNA substrates containing target sites.

A variety of methods are known for the introduction of nucleotide sequences and polypeptides into an organism, including, for example, transformation, sexual crossing, and the introduction of the polypeptide, DNA, or mRNA into the cell.

Methods for contacting, providing, and/or introducing a composition into various organisms are known and include but are not limited to, stable transformation methods, transient transformation methods, virus-mediated methods, and sexual breeding. Stable transformation indicates that the introduced polynucleotide integrates into the genome of the organism and is capable of being inherited by progeny thereof. Transient transformation indicates that the introduced composition is only temporarily expressed or present in the organism.

Protocols for introducing polynucleotides and polypeptides into plants may vary depending on the type of plant or plant cell targeted for transformation, such as monocot or dicot. Protocols for introducing polynucleotides, polypeptides or polynucleotide-protein complexes (PGEN, RGEN) into eukaryotic cells, such as plants or plant cells are known and include microinjection (Crossway et al., (1986) Biotechniques 4:320-34 and U.S. Pat. No. 6,300,543), meristem transformation (U.S. Pat. No. 5,736,369), electroporation (Riggs et al., (1986) Proc. Natl. Acad. Sci. USA 83:5602-6, Agrobacterium-mediated transformation (U.S. Pat. Nos. 5,563,055 and 5,981,840), direct gene transfer (Paszkowski et al., (1984) EMBO J 3:2717-22), and ballistic particle acceleration (U.S. Pat. Nos. 4,945,050; 5,879,918; 5,886,244; 5,932,782; Tomes et al., (1995) “Direct DNA Transfer into Intact Plant Cells via Microprojectile Bombardment” in Plant Cell, Tissue, and Organ Culture: Fundamental Methods, ed. Gamborg & Phillips (Springer-Verlag, Berlin); McCabe et al., (1988) Biotechnology 6:923-6; Weissinger et al., (1988) Ann Rev Genet 22:421-77; Sanford et al., (1987) Particulate Science and Technology 5:27-37 (onion); Christou et al., (1988) Plant Physiol 87:671-4 (soybean); Finer and McMullen, (1991) In Vitro Cell Dev Biol 27P:175-82 (soybean); Singh et al., (1998) Theor Appl Genet 96:319-24 (soybean); Datta et al., (1990) Biotechnology 8:736-40 (rice); Klein et al., (1988) Proc. Natl. Acad. Sci. USA 85:4305-9 (maize); Klein et al., (1988) Biotechnology 6:559-63 (maize); U.S. Pat. Nos. 5,240,855; 5,322,783 and 5,324,646; Klein et al., (1988) Plant Physiol 91:440-4 (maize); Fromm et al., (1990) Biotechnology 8:833-9 (maize); Hooykaas-Van Slogteren et al., (1984) Nature 311:763-4; U.S. Pat. No. 5,736,369 (cereals); Bytebier et al., (1987) Proc. Natl. Acad. Sci. USA 84:5345-9 (Liliaceae); De Wet et al., (1985) in The Experimental Manipulation of Ovule Tissues, ed. Chapman et al., (Longman, New York), pp. 197-209 (pollen); Kaeppler et al., (1990) Plant Cell Rep 9:415-8) and Kaeppler et al., (1992) Theor Appl Genet 84:560-6 (whisker-mediated transformation); D'Halluin et al., (1992) Plant Cell 4:1495-505 (electroporation); Li et al., (1993) Plant Cell Rep 12:250-5; Christou and Ford (1995) Annals Botany 75:407-13 (rice) and Osjoda et al., (1996) Nat Biotechnol 14:745-50 (maize via Agrobacterium tumefaciens).

Alternatively, polynucleotides may be introduced into plants by contacting plants with a virus or viral nucleic acids. Generally, such methods involve incorporating a polynucleotide within a viral DNA or RNA molecule. In some examples a polypeptide of interest may be initially synthesized as part of a viral polyprotein, which is later processed by proteolysis in vivo or in vitro to produce the desired recombinant protein. Methods for introducing polynucleotides into plants and expressing a protein encoded therein, involving viral DNA or RNA molecules, are known, see, for example, U.S. Pat. Nos. 5,889,191, 5,889,190, 5,866,785, 5,589,367 and 5,316,931. Transient transformation methods include, but are not limited to, the introduction of polypeptides, such as a double-strand break inducing agent, directly into the organism, the introduction of polynucleotides such as DNA and/or RNA polynucleotides, and the introduction of the RNA transcript, such as an mRNA encoding a double-strand break inducing agent, into the organism. Such methods include, for example, microinjection or particle bombardment. See, for example Crossway et al., (1986) Mol Gen Genet 202:179-85; Nomura et al., (1986) Plant Sci 44:53-8; Hepler et al., (1994) Proc. Natl. Acad. Sci. USA 91:2176-80; and, Hush et al., (1994) J Cell Sci 107:775-84.

The term “dicot” refers to the subclass of angiosperm plants also knows as “dicotyledoneae” and includes reference to whole plants, plant organs (e.g., leaves, stems, roots, etc.), seeds, plant cells, and progeny of the same. Plant cell, as used herein includes, without limitation, seeds, suspension cultures, embryos, meristematic regions, callus tissue, leaves, roots, shoots, gametophytes, sporophytes, pollen, and microspores.

The term “crossed” or “cross” or “crossing” in the context of this disclosure means the fusion of gametes via pollination to produce progeny (i.e., cells, seeds, or plants). The term encompasses both sexual crosses (the pollination of one plant by another) and selfing (self-pollination, i.e., when the pollen and ovule (or microspores and megaspores) are from the same plant or genetically identical plants).

The term “introgression” refers to the transmission of a desired allele of a genetic locus from one genetic background to another. For example, introgression of a desired allele at a specified locus can be transmitted to at least one progeny plant via a sexual cross between two parent plants, where at least one of the parent plants has the desired allele within its genome. Alternatively, for example, transmission of an allele can occur by recombination between two donor genomes, e.g., in a fused protoplast, where at least one of the donor protoplasts has the desired allele in its genome. The desired allele can be, e.g., a transgene, a modified (mutated or edited) native allele, or a selected allele of a marker or QTL.

Standard DNA isolation, purification, molecular cloning, vector construction, and verification/characterization methods are well established, see, for example Sambrook et al., (1989) Molecular Cloning: A Laboratory Manual, (Cold Spring Harbor Laboratory Press, NY). Vectors and constructs include circular plasmids, and linear polynucleotides, comprising a polynucleotide of interest and optionally other components including linkers, adapters, regulatory or analysis. In some examples a recognition site and/or target site can be contained within an intron, coding sequence, 5′ UTRs, 3′ UTRs, and/or regulatory regions.

The present disclosure further provides expression constructs for expressing in a plant, plant cell, or plant part a guide RNA/Cas9 system that is capable of binding to and creating a double strand break in a target site. In one embodiment, the expression constructs of the disclosure comprise a promoter operably linked to a nucleotide sequence encoding a Cas9 gene and a promoter operably linked to a guide RNA of the present disclosure. The promoter is capable of driving expression of an operably linked nucleotide sequence in a plant cell.

A phenotypic marker is a screenable or selectable marker that includes visual markers and selectable markers whether it is a positive or negative selectable marker. Any phenotypic marker can be used. Specifically, a selectable or screenable marker comprises a DNA segment that allows one to identify, or select for or against a molecule or a cell that contains it, often under particular conditions. These markers can encode an activity, such as, but not limited to, production of RNA, peptide, or protein, or can provide a binding site for RNA, peptides, proteins, inorganic and organic compounds or compositions and the like.

Examples of selectable markers include, but are not limited to, DNA segments that comprise restriction enzyme sites; DNA segments that encode products which provide resistance against otherwise toxic compounds including antibiotics, such as, spectinomycin, ampicillin, kanamycin, tetracycline, Basta, neomycin phosphotransferase II (NEO) and hygromycin phosphotransferase (HPT)); DNA segments that encode products which are otherwise lacking in the recipient cell (e.g., tRNA genes, auxotrophic markers); DNA segments that encode products which can be readily identified (e.g., phenotypic markers such β-galactosidase, GUS; fluorescent proteins such as green fluorescent protein (GFP), cyan (CFP), yellow (YFP), red (RFP), and cell surface proteins); the generation of new primer sites for PCR (e.g., the juxtaposition of two DNA sequence not previously juxtaposed), the inclusion of DNA sequences not acted upon or acted upon by a restriction endonuclease or other DNA modifying enzyme, chemical, etc.; and, the inclusion of a DNA sequences required for a specific modification (e.g., methylation) that allows its identification.

Additional selectable markers include genes that confer resistance to herbicidal compounds, such as glufosinate ammonium, bromoxynil, imidazolinones, and 2,4-dichlorophenoxyacetate (2,4-D). See for example, Yarranton, (1992) Curr Opin Biotech 3:506-11)

The cells having the introduced sequence may be grown or regenerated into plants using conventional conditions, see for example, McCormick et al., (1986) Plant Cell Rep 5:81-4. These plants may then be grown, and either pollinated with the same transformed strain or with a different transformed or untransformed strain, and the resulting progeny having the desired characteristic and/or comprising the introduced polynucleotide or polypeptide identified. Two or more generations may be grown to ensure that the polynucleotide is stably maintained and inherited, and seeds harvested.

Any plant can be used, including monocot and dicot plants. Examples of monocot plants that can be used include, but are not limited to, corn (Zea mays), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), wheat (Triticum aestivum), sugarcane (Saccharum spp.), oats (Avena), barley (Hordeum), switchgrass (Panicum virgatum), pineapple (Ananas comosus), banana (Musa spp.), palm, ornamentals, turfgrasses, and other grasses. Examples of dicot plants that can be used include, but are not limited to, soybean (Glycine max), canola (Brassica napus and B. campestris), alfalfa (Medicago sativa), tobacco (Nicotiana tabacum), Arabidopsis (Arabidopsis thaliana), sunflower (Helianthus annuus), cotton (Gossypium arboreum), and peanut (Arachis hypogaea), tomato (Solanum lycopersicum), potato (Solanum tuberosum) etc.

The transgenes, recombinant DNA molecules, DNA sequences of interest, and polynucleotides of interest can comprise one or more genes of interest. Such genes of interest can encode, for example, a protein that provides agronomic advantage to the plant.

The meaning of abbreviations is as follows: “sec” means second(s), “min” means minute(s), “h” means hour(s), “d” means day(s), “A” means microliter(s), “mL” means milliliter(s), “L” means liter(s), “μM” means micromolar, “mM” means millimolar, “M” means molar, “mmol” means millimole(s), “μmole” mean micromole(s), “g” means gram(s), “μg” means microgram(s), “ng” means nanogram(s), “U” means unit(s), “bp” means base pair(s) and “kb” means kilobase(s).
Non-limiting examples of compositions and methods disclosed herein are as follows:
1. A single guide RNA capable of forming a guide RNA/Cas9 endonuclease complex, wherein said guide RNA/Cas9 endonuclease complex can recognize, bind to, and optionally nick or cleave a target sequence, wherein said single guide RNA is selected from the group consisting of SEQ ID NOs: 185-207, a functional fragment of SEQ ID NOs: 185-207, and a functional variant of SEQ ID NOs: 185-207.
2. A single guide RNA capable of forming a guide RNA/Cas9 endonuclease complex, wherein said guide RNA/Cas9 endonuclease complex can recognize, bind to, and optionally nick or cleave a target sequence, wherein said single guide RNA comprises a chimeric non-naturally occurring crRNA linked to a tracrRNA, wherein said tracrRNA comprises a nucleotide sequence selected from the group consisting of SEQ ID NOs: 139-184, a functional fragment of SEQ ID NOs: 139-184, and a functional variant of SEQ ID NOs: 139-184.
3. A single guide RNA capable of forming a guide RNA/Cas9 endonuclease complex, wherein said guide RNA/Cas9 endonuclease complex can recognize, bind to, and optionally nick or cleave a target sequence, wherein said single guide RNA comprises a chimeric non-naturally occurring crRNA linked to a tracrRNA, wherein said chimeric non-naturally occurring crRNA comprises a nucleotide sequence selected from the group consisting of SEQ ID NOs: 116-138, a functional fragment of SEQ ID NOs: 116-138, and a functional variant of SEQ ID NOs: 116-138.
4. A guide RNA capable of forming a guide RNA/Cas9 endonuclease complex, wherein said guide RNA/Cas9 endonuclease complex can recognize, bind to, and optionally nick or cleave a target sequence, wherein said guide RNA is a duplex molecule comprising a chimeric non-naturally occurring crRNA and a tracrRNA, wherein said chimeric non-naturally occurring crRNA comprises a variable targeting domain capable of hybridizing to said target sequence, wherein said tracrRNA comprises a nucleotide sequence selected from the group consisting of SEQ ID NOs: 139-184, a functional fragment of SEQ ID NOs: 139-184, and a functional variant of SEQ ID NOs: 139-184, wherein said chimeric non-naturally occurring crRNA comprises a variable targeting domain capable of hybridizing to said target sequence.
5. A guide RNA capable of forming a guide RNA/Cas9 endonuclease complex, wherein said guide RNA/Cas9 endonuclease complex can recognize, bind to, and optionally nick or cleave a target sequence, wherein said guide RNA is a duplex molecule comprising a chimeric non-naturally occurring crRNA and a tracrRNA, wherein said chimeric non-naturally occurring crRNA comprises a nucleotide sequence selected from the group consisting of SEQ ID NOs: 116-138, a functional fragment of SEQ ID NOs: 116-138, and a functional variant of SEQ ID NOs: 116-138, wherein said chimeric non-naturally occurring crRNA comprises a variable targeting domain capable of hybridizing to said target sequence.
6. A guide RNA/Cas9 endonuclease complex comprising a Cas9 endonuclease selected from the group consisting of SEQ ID NOs: 47-69, a functional fragment of SEQ ID NOs: 47-69, and a functional variant of SEQ ID NOs: 47-69, and at least one guide RNA, wherein said guide RNA/Cas9 endonuclease complex is capable of recognizing, binding to, and optionally nicking or cleaving all or part of a target sequence.
7. A guide RNA/Cas9 endonuclease complex comprising at least one guide RNA and a Cas9 endonuclease, wherein said Cas9 endonuclease is encoded by a DNA sequence selected from the group consisting of SEQ ID NOs: 24-46, a functional fragment of SEQ ID NOs: 24-46, and a functional variant of SEQ ID NOs: 24-46, wherein said guide RNA/Cas9 endonuclease complex is capable of recognizing, binding to, and optionally nicking or cleaving all or part of a target sequence.
8. The guide RNA/Cas9 endonuclease complex of any of embodiments 6-7 comprising at least one guide RNA of any one of embodiments 1-5.
9. The guide RNA/Cas9 endonuclease complex of any of embodiments 6-7, wherein said target sequence is located in the genome of a cell.
10. A method for modifying a target site in the genome of a cell, the method comprising introducing into said cell at least one guide RNA and at least one Cas9 endonuclease selected from the group consisting of SEQ ID NOs: 47-69, a functional fragment of SEQ ID NOs: 47-69, and a functional variant of SEQ ID NOs: 47-69, wherein said guide RNA and Cas9 endonuclease can form a complex that is capable of recognizing, binding to, and optionally nicking or cleaving all or part of said target site.
11. The method of embodiment 10, further comprising identifying at least one cell that has a modification at said target, wherein the modification at said target site is selected from the group consisting of (i) a replacement of at least one nucleotide, (ii) a deletion of at least one nucleotide, (iii) an insertion of at least one nucleotide, and (iv) any combination of (i)-(iii).
12. A method for editing a nucleotide sequence in the genome of a cell, the method comprising introducing into said cell a polynucleotide modification template, at least one guide RNA and at least one Cas9 endonuclease selected from the group consisting of SEQ ID NOs: 47-69, a functional fragment of SEQ ID NOs: 47-69, and a functional variant of SEQ ID NOs: 47-69, wherein said polynucleotide modification template comprises at least one nucleotide modification of said nucleotide sequence, wherein said guide RNA and Cas9 endonuclease can form a complex that is capable of recognizing, binding to, and optionally nicking or cleaving all or part of said target site.
13. A method for modifying a target site in the genome of a cell, the method comprising introducing into said cell at least one guide RNA, at least one donor DNA, and at least one Cas9 endonuclease selected from the group consisting of SEQ ID NOs: 47-69, a functional fragment of SEQ ID NOs: 47-69, and a functional variant of SEQ ID NOs: 47-69, wherein said at least one guide RNA and at least one Cas9 endonuclease can form a complex that is capable of recognizing, binding to, and optionally nicking or cleaving all or part of said target site, wherein said donor DNA comprises a polynucleotide of interest.
14. The method of embodiment 13, further comprising identifying at least one cell that has said polynucleotide of interest integrated in or near said target site.
15. The method of any one of embodiments 10-14, wherein the cell is selected from the group consisting of a human, non-human, animal, bacterial, fungal, insect, yeast, non-conventional yeast, and plant cell.
16. The method of embodiment 15, wherein the plant cell is selected from the group consisting of a monocot and dicot cell.
17. The method of embodiment 16, wherein the plant cell is selected from the group consisting of maize, rice, sorghum, rye, barley, wheat, millet, oats, sugarcane, turfgrass, or switchgrass, soybean, canola, alfalfa, sunflower, cotton, tobacco, peanut, potato, tobacco, Arabidopsis, and safflower cell.
18. A plant comprising a modified target site, wherein said plant originates from a plant cell comprising a modified target site produced by the method of any of embodiments 10-17.
19. A plant comprising an edited nucleotide, wherein said plant originates from a plant cell comprising an edited nucleotide produced by the method of embodiment 12.

### EXAMPLES

In the following Examples, unless otherwise stated, parts and percentages are by weight and degrees are Celsius. It should be understood that these Examples, while indicating embodiments of the disclosure, are given by way of illustration only. From the above discussion and these Examples, one skilled in the art can make various changes and modifications of the disclosure to adapt it to various usages and conditions. Such modifications are also intended to fall within the scope of the appended claims.

### Example 1

### Characterization of New Cas9 Endonucleases and Cognate Guide RNAs

CRISPR-Cas loci from uncharacterized Type II CRISPR-Cas systems were identified by searching internal Pioneer-DuPont databases consisting of microbial genomes as described below. First, multiple sequence alignment of protein sequences from a diverse collection of Cas9 endonucleases was performed using MUSCLE (Edgar R. (2004) Nucleic Acids Res. 32(5): 1792-97). The alignments were examined and curated and were used to build profile hidden Markov models (HMM) for Cas9 sub-families using HMMER (Eddy S. R. (1998) Bioinformatics 14:755-763; Eddy S. R. (2011) PLoS Comp. Biol., 7:e1002195). The resulting HMM models were then utilized to search protein sequences translated from DNA sequence collections for the presence of cas9-like genes. The resulting genes were further validated as encoding a Cas9 protein by examining the translated amino acid sequence for the presence of HNH and RuvC cleavage domains. To further validate the gene as encoding a Cas9 protein, the other structural components of a Type II CRISPR-Cas system (Makarova et al. (2015) Nat. Rev. Microbiol. 13:722-736) (cas1 gene, cas2 gene, CRISPR array and tracrRNA encoding region) were identified in the DNA locus. Cas1 and cas2 genes were identified by examining the protein translations of open-reading-frames (ORFs) 201 nucleotides within the CRISPR-Cas locus against the NCBI protein database for those matching known Cas1 and Cas2 proteins using the PSI-BLAST program (Altschul, S. F. et al. (2005) FEBS J. 272:5101-5109.). The CRISPR array was detected using the PILER-CR program (Edgar R. (2007) BMC Bioinformatics 8:18. Additional CRISPR array repeats not detected by PILER-CR were identified by performing pairwise alignments of the locus with the PILER-CR identified repeats using the blastn program (Altschul, S. F. et al. (1997) Nucleic Acids Res. 25:3389-3402). The tracrRNA encoding region, termed the anti-repeat, was established by searching the locus for regions (distinct from the CRISPR array) with complete to partial homology to the repeats in the CRISPR array. In total, 23 DNA regions (Locus 6 to Locus 28, respectively) were selected (Table 2).

TABLE 2
List of sequence for Type 11 CRISPR-Cas
loci identified from Pioneer-Dupont databases.

| CRISPR-Cas | | |
|---|---|---|
| locus name | Genus/species of Origin | SEQ ID NO: |
| Locus 6 | Bacillus cereus | 1 |
| Locus 7 | Brevibacillus laterosporus | 2 |
| Locus 8 | Bacillus species | 3 |
| Locus 9 | Bacillus cereus | 4 |
| Locus 10 | Lactobacillus fermentum | 5 |
| Locus 11 | Enterococcus faecalis | 6 |
| Locus 12 | Bacillus cereus | 7 |
| Locus 13 | Enterococcus faecalis | 8 |
| Locus 14 | Unknown | 9 |
| Locus 15 | Enterococcus faecalis | 10 |
| Locus 16 | Metagenomic | 11 |
| Locus 17 | Chryseobacterium species | 12 |
| Locus 18 | Metagenomic | 13 |
| Locus 19 | Metagenomic | 14 |
| Locus 20 | Unknown | 15 |
| Locus 21 | Metagenomic | 16 |
| Locus 22 | Metagenomic | 17 |
| Locus 23 | Metagenomic | 18 |
| Locus 24 | Metagenomic | 19 |
| Locus 25 | Metagenomic | 20 |
| Locus 26 | Metagenomic | 21 |
| Locus 27 | Metacenomic | 22 |
| Locus 28 | Metagenomic | 23 |

A schematic of the DNA locus for each system is depicted in FIGS. 1-23. The cas9 gene open-reading-frame (cas9 gene ORF), accessory protein gene ORF (e.g. Cas1, Cas2, and when present Csn2), CRISPR array with CRISPR repeats, and anti-repeat (the genomic DNA region demonstrating partial homology to the CRISPR array repeat that indicates the location of the encoded tracrRNA) are indicated.

The genomic DNA sequence and length of each cas9 gene ORF and cas9 gene translation (not including the stop codon) are referenced in Table 3 for each system. Table 4 lists the consensus sequence of the CRISPR array repeats from the DNA locus of each system and the sequences of the anti-repeat for each system (as DNA sequence on the same strand as the cas9 gene ORF).

TABLE 3
Sequence and length of the cas9 gene ORF and cas9 gene translation
from each Type II CRISPR-Cas system identified as described herein.

| | | | | Length of |
|---|---|---|---|---|
| | | | Translation of | cas9 Gene |
| | | | cas9 Gene ORF | Translation |
| Cas9 | | Length of | (not including | (No. of |
| endonuclease | cas9 Gene ORF | cas9 Gene | the stop codon) | Amino |
| name | (SEQ ID NO:) | ORF (bp) | (SEQ ID NO) | Acids) |
| Cas-Locus 6 | 24 | 3282 | 47 | 1093 |
| Cas-Locus 7 | 25 | 3279 | 48 | 1092 |
| Cas-Locus 8 | 26 | 3213 | 49 | 1070 |
| Cas-Locus 9 | 27 | 3246 | 50 | 1081 |
| Cas-Locus 10 | 28 | 4137 | 51 | 1378 |
| Cas-Locus 11 | 29 | 4014 | 52 | 1337 |
| Cas-Locus 12 | 30 | 4014 | 53 | 1337 |
| Cas-Locus 13 | 31 | 4014 | 54 | 1337 |
| Cas-Locus 14 | 32 | 3993 | 55 | 1330 |
| Cas-Locus 15 | 33 | 3453 | 56 | 1150 |
| Cas-Locus 16 | 34 | 4371 | 57 | 1456 |
| Cas-Locus 17 | 35 | 4389 | 58 | 1462 |
| Cas-Locus 18 | 36 | 4323 | 59 | 1440 |
| Cas-Locus 19 | 37 | 4323 | 60 | 1440 |
| Cas-Locus 20 | 38 | 4323 | 61 | 1440 |
| Cas-Locus 21 | 39 | 3702 | 62 | 1233 |
| Cas-Locus 22 | 40 | 3807 | 63 | 1268 |
| Cas-Locus 23 | 41 | 3795 | 64 | 1264 |
| Cas-Locus 24 | 42 | 4395 | 65 | 1464 |
| Cas-Locus 25 | 43 | 4377 | 66 | 1458 |
| Cas-Locus 26 | 44 | 3384 | 67 | 1127 |
| Cas-Locus 27 | 45 | 3327 | 68 | 1108 |
| Cas-Locus 28 | 46 | 3327 | 69 | 1108 |

TABLE 4
CRISPR repeat consensus and anti-repeat (putative tracrRNA coding
region) for diverse Type II CRISPR-Cas systems described herein.

| | CRISPR | | | Anti- | |
|---|---|---|---|---|---|
| | repeat | | | Repeat | |
| CRISPR- | consensus | CRISPR | CRISPR Array | Consensus | Anti- |
| Cas locus | (SEQ | repeat | Transcriptional | (SEQ | Repeat |
| name | ID NO) | length | Direction | ID NO) | Direction |
| Locus 6 | 70 | 36 | Anti-sense | 93 | Sense |
| Locus 7 | 71 | 36 | Anti-sense | 94 | Sense |
| Locus 8 | 72 | 36 | Anti-sense | 95 | Sense |
| Locus 9 | 73 | 36 | Anti-sense | 96 | Sense |
| Locus 10 | 74 | 36 | Sense | 97 | Anti-sense |
| Locus 11 | 75 | 36 | Sense | 98 | Anti-sense |
| Locus 12 | 76 | 36 | Sense | 99 | Anti-sense |
| Locus 13 | 77 | 36 | Sense | 100 | Anti-sense |
| Locus 14 | 78 | 36 | Sense | 101 | Anti-sense |
| Locus 15 | 79 | 36 | Sense | 102 | Sense |
| Locus 16 | 80 | 47 | Anti-sense | 103 | Anti-sense |
| Locus 17 | 81 | 47 | Anti-sense | 104 | Anti-sense |
| Locus 18 | 82 | 47 | Anti-sense | 105 | Anti-sense |
| Locus 19 | 83 | 47 | Anti-sense | 106 | Anti-sense |
| Locus 20 | 84 | 47 | Anti-sense | 107 | Anti-sense |
| Locus 21 | 85 | 47 | Anti-sense | 108 | Anti-sense |
| Locus 22 | 86 | 47 | Anti-sense | 109 | Anti-sense |
| Locus 23 | 87 | 46 | Anti-sense | 110 | Anti-sense |
| Locus 24 | 88 | 36 | Sense | 111 | Anti-sense |
| Locus 25 | 89 | 36 | Sense | 112 | Anti-sense |
| Locus 26 | 90 | 36 | Sense | 113 | Anti-sense |
| Locus 27 | 91 | 36 | Anti-sense | 114 | Anti-sense |
| Locus 28 | 92 | 36 | Anti-sense | 115 | Anti-sense |

The possible transcriptional directions of the putative tracrRNAs for each new system were considered by examining the secondary structures and possible termination signals present in a RNA version of the sense and anti-sense genomic DNA sequences surrounding the anti-repeat (as described in U.S. patent applications 62/162,377 filed May 15, 2015, 62/162,353 filed May 15, 2015 and 62/196,535 filed Jul. 24, 2015, all three applications incorporated in their entirety herein by reference). Based on the hairpin-like secondary structures and termination signals present for each system, the transcriptional direction of the tracrRNA for all the Type II CRISPR-Cas systems can be deduced. Because the anti-repeat in the tracrRNA can hybridize to the crRNA derived from the CRISPR array to form a duplexed RNA capable of guiding the Cas9 endonuclease to cleave invading DNA the transcriptional direction of the CRISPR array may also be determined based on the direction of tracrRNA transcription (since double-stranded RNA hybridizes with 5′ to 3′ directionality). The transcriptional directions of both the tracrRNA and CRISPR array were deduced for each system as described above and are listed in Table 4 and depicted in FIGS. 1-23. Based on the likely transcriptional direction of the tracrRNA and CRISPR array, single guide RNAs (sgRNAs, SEQ ID NOs: 185-207) were designed and are shown in Table 5.

TABLE 5
Examples of sgRNAs (SEQ ID NOs: 185-207)
and its components (VT, crRNA repeat, loop,
anti-repeat and 3′tracrRNA) for each new diverse
Type II CRISPR-Cas endonuclease described herein.

| | | | | | | Single |
|---|---|---|---|---|---|---|
| | | crRNA | | Anti- | 3′tracr | guide |
| Cas | Variable | repeat | | Repeat | RNA | RNA |
| endo- | targeting | (SEQ | | (SEQ | (SEQ | (sgRNA) |
| nuclease | domain | ID | | ID | ID | SEQ |
| name | (VT) | NO) | Loop | NO) | NO) | ID NO: |
| Cas-Locus 6 | N_{20 (*)} | 116 | N_{4 (**)} | 139 | 162 | 185 |
| Cas-Locus 7 | N_{20 (*)} | 117 | N_{4 (**)} | 140 | 163 | 186 |
| Cas-Locus 8 | N_{20 (*)} | 118 | N_{4 (**)} | 141 | 164 | 187 |
| Cas-Locus 9 | N_{20 (*)} | 119 | N_{4 (**)} | 142 | 165 | 188 |
| Cas-Locus 10 | N_{20 (*)} | 120 | N_{4 (**)} | 143 | 166 | 189 |
| Cas-Locus 11 | N_{20 (*)} | 121 | N_{4 (**)} | 144 | 167 | 190 |
| Cas-Locus 12 | N_{20 (*)} | 122 | N_{4 (**)} | 145 | 168 | 191 |
| Cas-Locus 13 | N_{20 (*)} | 123 | N_{4 (**)} | 146 | 169 | 192 |
| Cas-Locus 14 | N_{20 (*)} | 124 | N_{4 (**)} | 147 | 170 | 193 |
| Cas-Locus 15 | N_{20 (*)} | 125 | N_{4 (**)} | 148 | 171 | 194 |
| Cas-Locus 16 | N_{20 (*)} | 126 | N_{4 (**)} | 149 | 172 | 195 |
| Cas-Locus 17 | N_{20 (*)} | 127 | N_{4 (**)} | 150 | 173 | 196 |
| Cas-Locus 18 | N_{20 (*)} | 128 | N_{4 (**)} | 151 | 174 | 197 |
| Cas-Locus 19 | N_{20 (*)} | 129 | N_{4 (**)} | 152 | 175 | 198 |
| Cas-Locus 20 | N_{20 (*)} | 130 | N_{4 (**)} | 153 | 176 | 199 |
| Cas-Locus 21 | N_{20 (*)} | 131 | N_{4 (**)} | 154 | 177 | 200 |
| Cas-Locus 22 | N_{20 (*)} | 132 | N_{4 (**)} | 155 | 178 | 201 |
| Cas-Locus 23 | N_{20 (*)} | 133 | N_{4 (**)} | 156 | 179 | 202 |
| Cas-Locus 24 | N_{20 (*)} | 134 | N_{4 (**)} | 157 | 180 | 203 |
| Cas-Locus 25 | N_{20 (*)} | 135 | N_{4 (**)} | 158 | 181 | 204 |
| Cas-Locus 26 | N_{20 (*)} | 136 | N_{4 (**)} | 159 | 182 | 205 |
| Cas-Locus 27 | N_{20 (*)} | 137 | N_{4 (**)} | 160 | 183 | 206 |
| Cas-Locus 28 | N_{20 (*)} | 138 | N_{4 (**)} | 161 | 184 | 207 |

N_{20 (*)} indicates a series of 20 nucleotides as one example of a sg RNA variable targeting domain. As described herein, the variable targeting domain of a sgRNA can vary for example, but not limiting from at least 12 to 30 nucleotides.
N_{4 (**)} indicates a loop of 4 nucleotides such as but not limiting to GAAA. As described herein, the length of the loop can vary from at least 3 nucleotides to 100 nucleotides.

Rapid in vitro methods to characterize the protospacer adjacent motif (PAM) specificity of Type II Cas9 proteins have been described (see U.S. patent applications 62/162,377 filed May 15, 2015, 62/162,353 filed May 15, 2015 and 62/196,535 filed Jul. 24, 2015, incorporated in their entirety herein by reference) and can be used to characterize the PAM preference of the novel CRISPR-Cas systems described herein.

The single guide RNAs described herein (Table 5) can be complexed with the respective purified Cas9 protein (for example SEQ ID NO: 185—Table 5—can be complexed with the Cas-Locus 6 endonuclease protein of SEQ ID NO: 47—Table 3) and assayed for their ability to support cleavage of a randomized PAM plasmid DNA library (as described in Example 7 of U.S. patent application 62/162,377 filed May 15, 2015). If the sgRNA does not support cleavage activity, new guide RNA designs (either sgRNA or duplexed crRNA and tracrRNA; in both possible transcriptional directions of the CRISPR array and anti-repeat region) will be tested for their ability to support cleavage.

Once a guide RNA that supports Cas9 cleavage has been established, the PAM specificity of each Cas9 endonuclease can be assayed (as described in Examples 4, 8, 14 and 15 U.S. patent application 62/162,377 filed May 15, 2015). PAM preferences which extend past the randomized PAM region may also be examined (as described in Example 11 U.S. patent application 62/162,377 filed May 15, 2015). After PAM preferences have been determined, the sgRNAs may be further refined for maximal activity or cellular transcription by either increasing or decreasing the tracrRNA 3′ end tail length, increasing or decreasing crRNA repeat and tracrRNA anti-repeat length, modifying the 4 nt self-folding loop or altering the sequence composition. The guide RNA solutions provided in Table 5 supported target recognition and cleavage for all of the Type II Cas9s examined (Cas-Locus 9, Cas-Locus 14, Cas-Locus 15, Cas-Locus 24, Cas-Locus 26, Cas-Locus 27 and Cas-Locus 28). Digestion of randomized PAM libraries followed by the capture and analysis of the PAM sequences which supported cleavage activity as described previously (see Examples 4, 8, 14 and 15 from U.S. patent application 62/162,377 filed May 15) yielded the PAM recognition profiles shown in Tables 6-12.

Taken together, the Type II Cas9 proteins combined with the guide polynucleotide solutions listed in Table 5 were capable of programmable RNA directed DNA target recognition and cleavage.

### Example 2

### Identification of Amino Acid Domains of Novel Cas9 Systems of the Present Disclosure

Multiple functional domains and conserved elements were determined for each of the novel Cas9 endonuclease proteins of the present disclosure. Tables 13-14 show the domain location of the HNH, RuvC-I, RuvC-II, RuvC-III, REC1, REC1′, REC-2, Bridge-Helix (BH) and PAM interacting (PI) domains along the amino acid sequence of each Cas9 endonuclease.

The novel Cas9 endonucleases of the present disclosure comprised an HNH domain, an RuvC domain that included three subdomains (RuvC-I, Ruvc-II and RuvC-II), a Brige Helicx domain a PAM interacting domain and DNA/RNA recognition regions including REC1 and REC1′. The REC1 binds to repeat:anti-repeat RNA duplex of the guide RNA while REC1′ mainly interacts with targetDNA:guide RNA hybrid duplex. The REC2 domain is a conserved element.

TABLE 13
Location of RuvC-I, BH, REC1 and REC2 domains of
novel Cas9 endonucleases of the present disclosure relative
to their respective Cas9 amino acid sequence.

| | Length of AA | | | | |
|---|---|---|---|---|---|
| Cas9 | sequence | RuvC-I | BH | REC1 | REC2 |
| Locus-6 | 1093 | 1-41 | 42-81 | 82-233 | None |
| (SEQ ID NO: 47) | | | | | |
| Locus-7 | 1092 | 1-41 | 42-81 | 82-232 | None |
| (SEQ ID NO: 48) | | | | | |
| Locus-8 | 1070 | 1-53 | 54-93 | 94-245 | None |
| (SEQ ID NO: 49) | | | | | |
| Locus-9 | 1081 | 1-41 | 42-81 | 82-231 | None |
| (SEQ ID NO: 50) | | | | | |
| Locus-10 | 1378 | 1-45 | 46-80 | 81-176 | 177-332 |
| (SEQ ID NO: 51) | | | | | |
| Locus-11 | 1337 | 1-58 | 59-93 | 94-176 | 177-312 |
| (SEQ ID NO: 52) | | | | | |
| Locus-12 | 1337 | 1-58 | 59-93 | 94-176 | 177-312 |
| (SEQ ID NO: 53) | | | | | |
| Locus-13 | 1337 | 1-58 | 59-93 | 94-176 | 177-312 |
| (SEQ ID NO: 54) | | | | | |
| Locus-14 | 1330 | 1-58 | 59-97 | 98-180 | 181-310 |
| (SEQ ID NO: 55) | | | | | |
| Locus-15 | 1150 | 1-40 | 41-75 | 76-257 | None |
| (SEQ ID NO: 56) | | | | | |
| Locus-16 | 1456 | 1-68 | 69-114 | 115-409 | undefined |
| (SEQ ID NO: 57) | | | | | |
| Locus-17 | 1462 | 1-80 | 81-126 | 127-431 | undefined |
| (SEQ ID NO: 58) | | | | | |
| Locus-18 | 1440 | 1-45 | 46-91 | 92-396 | undefined |
| (SEQ ID NO: 59) | | | | | |
| Locus-19 | 1440 | 1-45 | 46-91 | 92-396 | undefined |
| (SEQ ID NO: 60) | | | | | |
| Locus-20 | 1440 | 1-45 | 46-91 | 92-396 | undefined |
| (SEQ ID NO: 61) | | | | | |
| Locus-21 | 1233 | 1-13* | 14-59 | 60-207 | None |
| (SEQ ID NO: 62) | | | | | |
| Locus-22 | 1268 | 1-47 | 47-93 | 94-244 | None |
| (SEQ ID NO: 63) | | | | | |
| Locus-23 | 1264 | 1-45 | 46-91 | 92-239 | None |
| (SEQ ID NO: 64) | | | | | |
| Locus-24 | 1464 | 1-43 | 44-85 | 86-346 | undefined |
| (SEQ ID NO: 65) | | | | | |
| Locus-25 | 1458 | 1-43 | 44-85 | 86-347 | undefined |
| (SEQ ID NO: 66) | | | | | |
| Locus-26 | | | | | |
| (SEQ ID NO: 67) | 1127 | 1-44 | 42-85 | 86-262 | None |
| Locus-27 | 1108 | 1-41 | 45-86 | 87-241 | None |
| (SEQ ID NO: 68) | | | | | |
| Locus-28 | | | | | |
| (SEQ ID NO: 69) | 1108 | 1-44 | 45-86 | 87-241 | None |

*This RuvC domain is missing a N-terminal fragment

TABLE 14
Location of REC1', RuvC-II, HNH, RuvC-III and PAM interacting (PI)
domains of novel Cas9 endonucleases of the present disclosure relative
to their respective Cas9 amino acid sequence.

| Cas9 | REC1' | RuvC-II | HNH | RuvC-III | PI |
|---|---|---|---|---|---|
| Locus-6 | 234-463 | 464-508 | 509-664 | 665-810 | 811-1093 |
| (SEQ ID NO: 47) | | | | | |
| Locus-7 | 233-462 | 463-707 | 508-663 | 664-809 | 810-1092 |
| (SEQ ID NO: 48) | | | | | |
| Locus-8 | 246-473 | 474-519 | 520-683 | 684-808 | 809-1070 |
| (SEQ ID NO: 49) | | | | | |
| Locus-9 | 231-460 | 461-505 | 506-660 | 661-808 | 809-1081 |
| (SEQ ID NO: 50) | | | | | |
| Locus-10 | 333-748 | 749-796 | 750-944 | 945-1101 | 1102-1378 |
| (SEQ ID NO: Si) | | | | | |
| Locus-11 | 313-729 | 730-777 | 778-930 | 931-1084 | 1085-1337 |
| (SEQ ID NO: 52) | | | | | |
| Locus-12 | 313-729 | 730-777 | 778-930 | 931-1084 | 1085-1337 |
| (SEQ ID NO: 53) | | | | | |
| Locus-13 | 313-729 | 730-777 | 778-930 | 931-1084 | 1085-1337 |
| (SEQ ID NO: 54) | | | | | |
| Locus-14 | 311-719 | 720-770 | 771-922 | 923-1104 | 1105-1330 |
| (SEQ ID NO: 55) | | | | | |
| Locus-15 | 258-470 | 471-517 | 517-690 | 691-848 | 849-1150 |
| (SEQ ID NO: 56) | | | | | |
| Locus-16 | undefined | 691-743 | 744-950 | 921-1178 | 1179-1456 |
| (SEQ ID NO: 57) | | | | | |
| Locus-17 | undefined | 711-764 | 765-971 | 972-1199 | 1200-1462 |
| (SEQ ID NO: 58) | | | | | |
| Locus-18 | undefined | 677-729 | 720-936 | 936-1164 | 1165-1440 |
| (SEQ ID NO: 59) | | | | | |
| Locus-19 | undefined | 677-729 | 720-936 | 936-1164 | 1165-1440 |
| (SEQ ID NO: 60) | | | | | |
| Locus-20 | undefined | 677-729 | 720-936 | 936-1164 | 1165-1440 |
| (SEQ ID NO: 61) | | | | | |
| Locus-21 | 208-474 | 475-521 | 522-704 | 705-892 | 893-1233 |
| (SEQ ID NO: 62) | | | | | |
| Locus-22 | 245-511 | 512-558 | 559-741 | 742-929 | 930-1268 |
| (SEQ ID NO: 63) | | | | | |
| Locus-23 | 240-506 | 507-553 | 554-736 | 737-924 | 925-1264 |
| (SEQ ID NO: 64) | | | | | |
| Locus-24 | undefined | 734-780 | 781-965 | 966-1163 | 1164-1464 |
| (SEQ ID NO: 65) | | | | | |
| Locus-25 | undefined | 728-774 | 775-969 | 970-1157 | 1158-1458 |
| (SEQ ID NO: 66) | | | | | |
| Locus-26 | 262-515 | 516-573 | 574-728 | 729-868 | 869-1127 |
| (SEQ ID NO: 67) | | | | | |
| Locus-27 | 242-497 | 498-543 | 543-718 | 719-886 | 887-1108 |
| (SEQ ID NO: 68) | | | | | |
| Locus-28 | 242-497 | 498-543 | 544-718 | 719-886 | 887-1108 |
| (SEQ ID NO: 69) | | | | | |

Length refers to the total amino acids of each Cas9 endonuclease protein.

The number range shown for each Cas9 endonuclease domain (RuvC-I, Bridge Helix (BH), REC1, REC2, REC1′, RuvC-II, HNH, RuvC-III and PAM interacting (PI)) indicates the location of the first amino acid and last amino acid of that domain relative to the amino acid sequence of its respective Cas9 endonuclease. For example, the RuvCI domain of Cas-Locus-6 (1-41) comprises 41 amino acids spanning from the first amino acid (amino acid 1) to the 41^{st} amino acid of the Cas9 endonuclease (Cas-Locus6) of SEQ ID NO: 11. None indicates that no REC2 domain is present is said Cas9 endonuclease.

### Example 3

### Transformation of Maize Immature Embryos

Transformation can be accomplished by various methods known to be effective in plants, including particle-mediated delivery, Agrobacterium-mediated transformation, PEG-mediated delivery, and electroporation.

a. Particle-mediated delivery

Transformation of maize immature embryos using particle delivery is performed as follows. Media recipes follow below.

The ears are husked and surface sterilized in 30% Clorox bleach plus 0.5% Micro detergent for 20 minutes, and rinsed two times with sterile water. The immature embryos are isolated and placed embryo axis side down (scutellum side up), 25 embryos per plate, on 560Y medium for 4 hours and then aligned within the 2.5-cm target zone in preparation for bombardment. Alternatively, isolated embryos are placed on 560L (Initiation medium) and placed in the dark at temperatures ranging from 26° C. to 37° C. for 8 to 24 hours prior to placing on 560Y for 4 hours at 26° C. prior to bombardment as described above.

Plasmids containing the double strand brake inducing agent and donor DNA are constructed using standard molecular biology techniques and co-bombarded with plasmids containing the developmental genes ODP2 (AP2 domain transcription factor ODP2 (Ovule development protein 2); US20090328252 A1) and Wushel (US2011/0167516).

The plasmids and DNA of interest are precipitated onto 0.6 μm (average diameter) gold pellets using a water-soluble cationic lipid transfection reagent as follows. DNA solution is prepared on ice using 1 μg of plasmid DNA and optionally other constructs for co-bombardment such as 50 ng (0.5 μl) of each plasmid containing the developmental genes ODP2 (AP2 domain transcription factor ODP2 (Ovule development protein 2); US20090328252 A1) and Wushel. To the pre-mixed DNA, 20 μl of prepared gold particles (15 mg/ml) and 5 μl of a water-soluble cationic lipid transfection reagent is added in water and mixed carefully. Gold particles are pelleted in a microfuge at 10,000 rpm for 1 min and supernatant is removed. The resulting pellet is carefully rinsed with 100 ml of 100% EtOH without resuspending the pellet and the EtOH rinse is carefully removed. 105 μl of 100% EtOH is added and the particles are resuspended by brief sonication. Then, 10 μl is spotted onto the center of each macrocarrier and allowed to dry about 2 minutes before bombardment.

Alternatively, the plasmids and DNA of interest are precipitated onto 1.1 μm (average diameter) tungsten pellets using a calcium chloride (CaCl₂) precipitation procedure by mixing 100 μl prepared tungsten particles in water, 10 μl (1 μg) DNA in Tris EDTA buffer (1 μg total DNA), 100 μl 2.5 M CaCl2, and 10 μl 0.1 M spermidine. Each reagent is added sequentially to the tungsten particle suspension, with mixing. The final mixture is sonicated briefly and allowed to incubate under constant vortexing for 10 minutes. After the precipitation period, the tubes are centrifuged briefly, liquid is removed, and the particles are washed with 500 ml 100% ethanol, followed by a 30 second centrifugation. Again, the liquid is removed, and 105 μl of 100% ethanol is added to the final tungsten particle pellet. For particle gun bombardment, the tungsten/DNA particles are briefly sonicated. 10 μl of the tungsten/DNA particles is spotted onto the center of each macrocarrier, after which the spotted particles are allowed to dry about 2 minutes before bombardment.

The sample plates are bombarded at level #4 with a Biorad Helium Gun. All samples receive a single shot at 450 PSI, with a total of ten aliquots taken from each tube of prepared particles/DNA.

Following bombardment, the embryos are incubated on 560P (maintenance medium) for 12 to 48 hours at temperatures ranging from 26C to 37C, and then placed at 26C. After 5 to 7 days the embryos are transferred to 560R selection medium containing 3 mg/liter Bialaphos, and subcultured every 2 weeks at 26C. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to a lighted culture room. Approximately 7-10 days later, developing plantlets are transferred to 272V hormone-free medium in tubes for 7-10 days until plantlets are well established. Plants are then transferred to inserts in flats (equivalent to a 2.5″ pot) containing potting soil and grown for 1 week in a growth chamber, subsequently grown an additional 1-2 weeks in the greenhouse, then transferred to Classic 600 pots (1.6 gallon) and grown to maturity. Plants are monitored and scored for transformation efficiency, and/or modification of regenerative capabilities.

Initiation medium (560L) comprises 4.0 g/l N6 basal salts (SIGMA C-1416), 1.0 ml/l Eriksson's Vitamin Mix (1000× SIGMA-1511), 0.5 mg/l thiamine HCl, 20.0 g/l sucrose, 1.0 mg/l 2,4-D, and 2.88 g/l L-proline (brought to volume with D-1 H2O following adjustment to pH 5.8 with KOH); 2.0 g/l Gelrite (added after bringing to volume with D-I H2O); and 8.5 mg/l silver nitrate (added after sterilizing the medium and cooling to room temperature).

Maintenance medium (560P) comprises 4.0 g/l N6 basal salts (SIGMA C-1416), 1.0 ml/l Eriksson's Vitamin Mix (1000× SIGMA-1511), 0.5 mg/l thiamine HCl, 30.0 g/l sucrose, 2.0 mg/l 2,4-D, and 0.69 g/l L-proline (brought to volume with D-I H2O following adjustment to pH 5.8 with KOH); 3.0 g/l Gelrite (added after bringing to volume with D-I H2O); and 0.85 mg/l silver nitrate (added after sterilizing the medium and cooling to room temperature).

Bombardment medium (560Y) comprises 4.0 g/l N6 basal salts (SIGMA C-1416), 1.0 ml/l Eriksson's Vitamin Mix (1000× SIGMA-1511), 0.5 mg/l thiamine HCl, 120.0 g/l sucrose, 1.0 mg/l 2,4-D, and 2.88 g/l L-proline (brought to volume with D-I H2O following adjustment to pH 5.8 with KOH); 2.0 g/l Gelrite (added after bringing to volume with D-I H2O); and 8.5 mg/l silver nitrate (added after sterilizing the medium and cooling to room temperature).

Selection medium (560R) comprises 4.0 g/l N6 basal salts (SIGMA C-1416), 1.0 ml/l Eriksson's Vitamin Mix (1000× SIGMA-1511), 0.5 mg/l thiamine HCl, 30.0 g/l sucrose, and 2.0 mg/l 2,4-D (brought to volume with D-I H2O following adjustment to pH 5.8 with KOH); 3.0 g/l Gelrite (added after bringing to volume with D-I H2O); and 0.85 mg/l silver nitrate and 3.0 mg/l bialaphos (both added after sterilizing the medium and cooling to room temperature).

Plant regeneration medium (288J) comprises 4.3 g/l MS salts (GIBCO 11117-074), 5.0 ml/l MS vitamins stock solution (0.100 g nicotinic acid, 0.02 g/l thiamine HCL, 0.10 g/l pyridoxine HCL, and 0.40 g/l glycine brought to volume with polished D-I H2O) (Murashige and Skoog (1962) Physiol. Plant. 15:473), 100 mg/l myo-inositol, 0.5 mg/l zeatin, 60 g/l sucrose, and 1.0 ml/l of 0.1 mM abscisic acid (brought to volume with polished D-I H2O after adjusting to pH 5.6); 3.0 g/l Gelrite (added after bringing to volume with D-I H2O); and 1.0 mg/l indoleacetic acid and 3.0 mg/l bialaphos (added after sterilizing the medium and cooling to 60° C.). Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL, and 0.40 g/l glycine brought to volume with polished D-I H2O), 0.1 g/l myo-inositol, and 40.0 g/l sucrose (brought to volume with polished D-I H2O after adjusting pH to 5.6); and 6 g/l bacto-agar (added after bringing to volume with polished D-I H2O), sterilized and cooled to 60° C.

b. Agrobacterium-mediated transformation

Agrobacterium-mediated transformation was performed essentially as described in Djukanovic et al. (2006) Plant Biotech J 4:345-57. Briefly, 10-12 day old immature embryos (0.8-2.5 mm in size) were dissected from sterilized kernels and placed into liquid medium (4.0 g/L N6 Basal Salts (Sigma C-1416), 1.0 ml/L Eriksson's Vitamin Mix (Sigma E-1511), 1.0 mg/L thiamine HCl, 1.5 mg/L 2, 4-D, 0.690 g/L L-proline, 68.5 g/L sucrose, 36.0 g/L glucose, pH 5.2). After embryo collection, the medium was replaced with 1 ml Agrobacterium at a concentration of 0.35-0.45 OD550. Maize embryos were incubated with Agrobacterium for 5 min at room temperature, then the mixture was poured onto a media plate containing 4.0 g/L N6 Basal Salts (Sigma C-1416), 1.0 ml/L Eriksson's Vitamin Mix (Sigma E-1511), 1.0 mg/L thiamine HCl, 1.5 mg/L 2, 4-D, 0.690 g/L L-proline, 30.0 g/L sucrose, 0.85 mg/L silver nitrate, 0.1 nM acetosyringone, and 3.0 g/L Gelrite, pH 5.8. Embryos were incubated axis down, in the dark for 3 days at 20° C., then incubated 4 days in the dark at 28° C., then transferred onto new media plates containing 4.0 g/L N6 Basal Salts (Sigma C-1416), 1.0 ml/L Eriksson's Vitamin Mix (Sigma E-1511), 1.0 mg/L thiamine HCl, 1.5 mg/L 2, 4-D, 0.69 g/L L-proline, 30.0 g/L sucrose, 0.5 g/L MES buffer, 0.85 mg/L silver nitrate, 3.0 mg/L Bialaphos, 100 mg/L carbenicillin, and 6.0 g/L agar, pH 5.8. Embryos were subcultured every three weeks until transgenic events were identified. Somatic embryogenesis was induced by transferring a small amount of tissue onto regeneration medium (4.3 g/L MS salts (Gibco 11117), 5.0 ml/L MS Vitamins Stock Solution, 100 mg/L myo-inositol, 0.1 μM ABA, 1 mg/L IAA, 0.5 mg/L zeatin, 60.0 g/L sucrose, 1.5 mg/L Bialaphos, 100 mg/L carbenicillin, 3.0 g/L Gelrite, pH 5.6) and incubation in the dark for two weeks at 28° C. All material with visible shoots and roots were transferred onto media containing 4.3 g/L MS salts (Gibco 11117), 5.0 ml/L MS Vitamins Stock Solution, 100 mg/L myo-inositol, 40.0 g/L sucrose, 1.5 g/L Gelrite, pH 5.6, and incubated under artificial light at 28° C. One week later, plantlets were moved into glass tubes containing the same medium and grown until they were sampled and/or transplanted into soil.

### Example 4

### Transient Expression of BBM Enhances Transformation

Parameters of the transformation protocol can be modified to ensure that the BBM activity is transient. One such method involves precipitating the BBM-containing plasmid in a manner that allows for transcription and expression, but precludes subsequent release of the DNA, for example, by using the chemical PEI. In one example, the BBM plasmid is precipitated onto gold particles with PEI, while the transgenic expression cassette (UBI:moPAT˜GFPm:PinII; moPAT is the maize optimized PAT gene) to be integrated is precipitated onto gold particles using the standard calcium chloride method.

Briefly, gold particles were coated with PEI as follows. First, the gold particles were washed. Thirty-five mg of gold particles, 1.0 in average diameter (A.S.I. #162-0010), were weighed out in a microcentrifuge tube, and 1.2 ml absolute EtOH was added and vortexed for one minute. The tube was incubated for 15 minutes at room temperature and then centrifuged at high speed using a microfuge for 15 minutes at 4oC. The supernatant was discarded and a fresh 1.2 ml aliquot of ethanol (EtOH) was added, vortexed for one minute, centrifuged for one minute, and the supernatant again discarded (this is repeated twice). A fresh 1.2 ml aliquot of EtOH was added, and this suspension (gold particles in EtOH) was stored at −20oC for weeks. To coat particles with polyethylimine (PEI; Sigma #P3143), 250 μl of the washed gold particle/EtOH mix was centrifuged and the EtOH discarded. The particles were washed once in 100 μl ddH2O to remove residual ethanol, 250 μl of 0.25 mM PEI was added, followed by a pulse-sonication to suspend the particles and then the tube was plunged into a dry ice/EtOH bath to flash-freeze the suspension, which was then lyophilized overnight. At this point, dry, coated particles could be stored at −80oC for at least 3 weeks. Before use, the particles were rinsed 3 times with 250 μl aliquots of 2.5 mM HEPES buffer, pH 7.1, with 1× pulse-sonication, and then a quick vortex before each centrifugation. The particles were then suspended in a final volume of 250 μl HEPES buffer. A 25 μl aliquot of the particles was added to fresh tubes before attaching DNA. To attach uncoated DNA, the particles were pulse-sonicated, then 1 μg of DNA (in 5 μl water) was added, followed by mixing by pipetting up and down a few times with a Pipetteman and incubated for 10 minutes. The particles were spun briefly (i.e. 10 seconds), the supernatant removed, and 60 μl EtOH added. The particles with PEI-precipitated DNA-1 were washed twice in 60 μl of EtOH. The particles were centrifuged, the supernatant discarded, and the particles were resuspended in 45 μl water. To attach the second DNA (DNA-2), precipitation using a water-soluble cationic lipid transfection reagent was used. The 45 μl of particles/DNA-1 suspension was briefly sonicated, and then 5 μl of 100 ng/μl of DNA-2 and 2.5 μl of the water-soluble cationic lipid transfection reagent were added. The solution was placed on a rotary shaker for 10 minutes, centrifuged at 10,000 g for 1 minute. The supernatant was removed, and the particles resuspended in 60 μl of EtOH. The solution was spotted onto macrocarriers and the gold particles onto which DNA-1 and DNA-2 had been sequentially attached were delivered into scutellar cells of 10 DAP Hi-II immature embryos using a standard protocol for the PDS-1000. For this experiment, the DNA-1 plasmid contained a UBI:RFP:pinII expression cassette, and DNA-2 contained a UBI:CFP:pinII expression cassette. Two days after bombardment, transient expression of both the CFP and RFP fluorescent markers was observed as numerous red & blue cells on the surface of the immature embryo. The embryos were then placed on non-selective culture medium and allowed to grow for 3 weeks before scoring for stable colonies. After this 3-week period, 10 multicellular, stably-expressing blue colonies were observed, in comparison to only one red colony. This demonstrated that PEI-precipitation could be used to effectively introduce DNA for transient expression while dramatically reducing integration of the PEI-introduced DNA and thus reducing the recovery of RFP-expressing transgenic events. In this manner, PEI-precipitation can be used to deliver transient expression of BBM and/or WUS2.

For example, the particles are first coated with UBI:BBM:pinII using PEI, then coated with UBI:moPAT˜YFP using a water-soluble cationic lipid transfection reagent, and then bombarded into scutellar cells on the surface of immature embryos. PEI-mediated precipitation results in a high frequency of transiently expressing cells on the surface of the immature embryo and extremely low frequencies of recovery of stable transformants Thus, it is expected that the PEI-precipitated BBM cassette expresses transiently and stimulates a burst of embryogenic growth on the bombarded surface of the tissue (i.e. the scutellar surface), but this plasmid will not integrate. The PAT˜GFP plasmid released from the Ca++/gold particles is expected to integrate and express the selectable marker at a frequency that results in substantially improved recovery of transgenic events. As a control treatment, PEI-precipitated particles containing a UBI:GUS:pinII (instead of BBM) are mixed with the PAT˜GFP/Ca++ particles. Immature embryos from both treatments are moved onto culture medium containing 3 mg/l bialaphos. After 6-8 weeks, it is expected that GFP+, bialaphos-resistant calli will be observed in the PEI/BBM treatment at a much higher frequency relative to the control treatment (PEI/GUS).

As an alternative method, the BBM plasmid is precipitated onto gold particles with PEI, and then introduced into scutellar cells on the surface of immature embryos, and subsequent transient expression of the BBM gene elicits a rapid proliferation of embryogenic growth. During this period of induced growth, the explants are treated with Agrobacterium using standard methods for maize (see Example 1), with T-DNA delivery into the cell introducing a transgenic expression cassette such as UBI:moPAT˜GFPm:pinII. After co-cultivation, explants are allowed to recover on normal culture medium, and then are moved onto culture medium containing 3 mg/l bialaphos. After 6-8 weeks, it is expected that GFP+, bialaphos-resistant calli will be observed in the PEI/BBM treatment at a much higher frequency relative to the control treatment (PEI/GUS).

It may be desirable to “kick start” callus growth by transiently expressing the BBM and/or WUS2 polynucleotide products. This can be done by delivering BBM and WUS2 5′-capped polyadenylated RNA, expression cassettes containing BBM and WUS2 DNA, or BBM and/or WUS2 proteins. All of these molecules can be delivered using a biolistics particle gun. For example 5′-capped polyadenylated BBM and/or WUS2 RNA can easily be made in vitro using Ambion's mMessage mMachine kit. RNA is co-delivered along with DNA containing a polynucleotide of interest and a marker used for selection/screening such as Ubi:moPAT˜GFPm:PinII. It is expected that the cells receiving the RNA will immediately begin dividing more rapidly and a large portion of these will have integrated the agronomic gene. These events can further be validated as being transgenic clonal colonies because they will also express the PAT˜GFP fusion protein (and thus will display green fluorescence under appropriate illumination). Plants regenerated from these embryos can then be screened for the presence of the polynucleotide of interest.

1. A synthetic composition comprising a target sequence and a single guide RNA capable of forming a guide RNA/Cas9 endonuclease complex, wherein said guide RNA/Cas9 endonuclease complex can recognize, bind to, and optionally nick or cleave the target sequence, wherein said single guide RNA comprises SEQ ID NO: 188, a functional fragment of SEQ ID NO: 188, or a functional variant of SEQ ID NO: 188.

2. A synthetic composition comprising a target sequence and a single guide RNA capable of forming a guide RNA/Cas9 endonuclease complex, wherein said guide RNA/Cas9 endonuclease complex can recognize, bind to, and optionally nick or cleave the target sequence, wherein said single guide RNA comprises a chimeric non-naturally occurring crRNA linked to a tracrRNA, wherein said tracrRNA comprises SEQ ID NO: 142 or 165, a functional fragment of SEQ ID NO: 142 or165, or a functional variant of SEQ ID NO: 142 or 165.

3. A synthetic composition comprising a target sequence and a single guide RNA capable of forming a guide RNA/Cas9 endonuclease complex, wherein said guide RNA/Cas9 endonuclease complex can recognize, bind to, and optionally nick or cleave the target sequence, wherein said single guide RNA comprises a chimeric non-naturally occurring crRNA linked to a tracrRNA, wherein said chimeric non-naturally occurring crRNA comprises SEQ ID NO: 119, a functional fragment of SEQ ID NO: 119, or a functional variant of SEQ ID NO: 119.

4. A synthetic composition comprising a target sequence and a guide RNA capable of forming a guide RNA/Cas9 endonuclease complex, wherein said guide RNA/Cas9 endonuclease complex can recognize, bind to, and optionally nick or cleave the target sequence, wherein said guide RNA is a duplex molecule comprising a chimeric non-naturally occurring crRNA and a tracrRNA, wherein said chimeric non-naturally occurring crRNA comprises a variable targeting domain capable of hybridizing to said target sequence, wherein said tracrRNA comprises SEQ ID NO: 142 or 165, a functional fragment of SEQ ID NO: 142 or165, or a functional variant of SEQ ID NO: 142 or 165; wherein said chimeric non-naturally occurring crRNA comprises a variable targeting domain capable of hybridizing to said target sequence.

5. A synthetic composition comprising a target sequence and a guide RNA capable of forming a guide RNA/Cas9 endonuclease complex, wherein said guide RNA/Cas9 endonuclease complex can recognize, bind to, and optionally nick or cleave the target sequence, wherein said guide RNA is a duplex molecule comprising a chimeric non-naturally occurring crRNA and a tracrRNA, wherein said chimeric non-naturally occurring crRNA comprises SEQ ID NO: 119, a functional fragment of SEQ ID NO: 119, or a functional variant of SEQ ID NO: 119; wherein said chimeric non-naturally occurring crRNA comprises a variable targeting domain capable of hybridizing to said target sequence.

6. A synthetic composition comprising a target sequence and a guide RNA/Cas9 endonuclease complex comprising a Cas9 endonuclease comprising SEQ ID NO: 50, a functional fragment of SEQ ID NO: 50, or a functional variant of SEQ ID NO: 50; and at least one guide RNA, wherein said guide RNA/Cas9 endonuclease complex is capable of recognizing, binding to, and optionally nicking or cleaving all or part of the target sequence.

7. A synthetic composition comprising a target sequence and a guide RNA/Cas9 endonuclease complex comprising at least one guide RNA and a Cas9 endonuclease, wherein said Cas9 endonuclease is encoded by a DNA sequence comprising SEQ ID NO: 27, a functional fragment of SEQ ID NO: 27, or a functional variant SEQ ID NO: 27, wherein said guide RNA/Cas9 endonuclease complex is capable of recognizing, binding to, and optionally nicking or cleaving all or part of the target sequence.

8. The synthetic composition of any of claims 6-7 wherein the guide RNA comprises a sequence selected from the group consisting of: SEQ ID NOs: 119, 142, 165, and 188.

9. The synthetic composition of any of claims 1-7, wherein said target sequence is located in the genome of a cell.

10. A method for modifying a target site in the genome of a cell, the method comprising introducing into said cell at least one guide RNA and at least one Cas9 endonuclease comprising SEQ ID NO: 50, a functional fragment of SEQ ID NO: 50, or a functional variant of SEQ ID NO: 50; wherein said guide RNA and Cas9 endonuclease can form a complex that is capable of recognizing, binding to, and optionally nicking or cleaving all or part of said target site; further comprising identifying at least one cell that has a modification at said target, wherein the modification at said target site is selected from the group consisting of (i) a replacement of at least one nucleotide, (ii) a deletion of at least one nucleotide, (iii) an insertion of at least one nucleotide, and (iv) any combination of (i)-(iii).

11. (canceled)

12. The method of claim 10, further comprising introducing into said cell a polynucleotide modification template, wherein said polynucleotide modification template comprises at least one nucleotide modification of said nucleotide sequence.

13. The method of claim 10, further comprising introducing into said cell at least one donor DNA, wherein said donor DNA comprises a polynucleotide of interest.

14. The method of claim 13, further comprising identifying at least one cell that has said polynucleotide of interest integrated in or near said target site.

15. The method of claim 10, wherein the cell is selected from the group consisting of a human, non-human, animal, bacterial, fungal, insect, yeast, non-conventional yeast, and plant cell.

16. The method of claim 15, wherein the plant cell is selected from the group consisting of a monocot and a dicot cell.

17. The method of claim 16, wherein the plant cell is selected from the group consisting of maize, rice, sorghum, rye, barley, wheat, millet, oats, sugarcane, turfgrass, or switchgrass, soybean, canola, alfalfa, sunflower, cotton, tobacco, peanut, potato, Arabidopsis, and safflower cell.

18. (canceled)

19. (canceled)
